(19) United States
(12) Patent Application Publication
(10) Pub. No.: US 2006/0052315 A1
(43) Pub. Date: Mar. 9, 2006

(54) Cb 1/cb 2 receptor ligands and their use in the treatment of pain

(75) Inventors: Carmen Leung, Montreal (CA); Miroslaw Tomaszewski, Montreal (CA); Simon Woo, Montreal (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 10/540,998

(22) PCT Filed: Dec. 29, 2003
(86) PCT No.: PCT/SE03/02088

§ 371 Date: Jun. 28, 2005

(30) Foreign Application Priority Data
Jan. 7, 2003 (SE) 03000106

(51) Int. Cl. A61K 38/04; A61K 31/47; A61K 31/381; A61K 31/277; A61K 31/138; A61K 31/137

(52) U.S. Cl. 514/19; 514/310; 514/649; 514/438; 514/521; 514/651

(57) ABSTRACT

Compounds of formula (I) or pharmaceutically acceptable salts thereof wherein Ar [symbol] 1?, Ar [symbol] 2?, R [symbol] 1?, R [symbol] 2?, n and X are as defined in the specifications well as salts and pharmaceutical compositions including the compounds are prepared. They are useful in therapy, in particular in the management of pain.

### BACKGROUND OF THE INVENTION

1. Field of the Invention

The invention is related to compounds which are CB₁/CB₂ receptor ligands, pharmaceutical compositions containg these compounds, manufacturing processes thereof and uses thereof, and more particularly to compounds that are CB₁/CB₂ receptor agonists. The present invention may also relate to compounds that may be effective in treating pain, cancer, multiple sclerosis, Parkinson's disease, Huntington's chorea, Alzheimer's disease, anxiety disorders, vision and/or eye related disorders, gastrointestinal disorders and cardiavascular disorders.

2. Discussion of Relevant Technology

Pain management has been an important field of study for many years. It has been well known that cannabinoid receptor (e.g., CB₁ receptors, CB₂ receptors) ligands, especially agonists produce relief of pain in a variety of animal models by interacting with CB₁ and/or CB₂ receptors. Generally, CB₁ receptors are located predominately in the central nervous system, whereas CB₂ receptors are located primarily in the periphery and are primarily restricted to the cells and tissues derived from the immune system.

While the conventional CB₁ receptor agonists and CB₁/CB₂ receptor agonists, such as tetrahydrocannabinol (THC) and opiate drugs, are highly effective in anti-nociception models in animals, they tend to exert many undesired CNS (central nerve system) side-effects, e.g., psychoactive side effects and the abuse potential of opiate drugs.

Therefore, there is a need for new CB₁/CB₂ receptor ligands such as agonists useful in managing pain or treating other related symptoms or diseases with reduced or minimal undesirable CNS side-effects. The compounds of the invention may be used to avoid the undesired CNS side effects which arise through the central CB1 mechanism.

### DISCLOSURE OF THE INVENTION

The present invention provides CB₁/CB₂ receptor ligands which are useful in treating pain and other related symptoms or diseases.

### DEFINITIONS

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

“CB₁/CB₂ receptors” means CB₁ and/or CB₂ receptors.

The term “Cₘ₋ₙ” or “Cₘ₋ₙ group” used alone or as a prefix, refers to any group having m to n carbon atoms, and having 0 to n multivalent heteroatoms selected from O, S, N and P, wherein m and n are 0 or positive integers, and n>m. For example, “C₁₋₆” would refer to a chemical group having 1 to 6 carbon atoms, and having 0 to 6 multivalent heteroatoms selected from O, S, N and P.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to monovalent straight or branched chain hydrocarbon radicals comprising 1 to about 12 carbon atoms. Unless otherwise specified, “alkyl” general includes both saturated alkyl and unsaturated alkyl.

The term “alkylene” used alone or as suffix or prefix, refers to divalent straight or branched chain hydrocarbon radicals comprising 1 to about 12 carbon atoms, which serves to links two structures together.

The term “alkenyl” used alone or as suffix or prefix, refers to a monovalent straight or branched chain hydrocarbon radical having at least one carbon-carbon double bond and comprising at least 2 up to about 12 carbon atoms.

The term “alkynyl” used alone or as suffix or prefix, refers to a monovalent straight or branched chain hydrocarbon radical having at least one carbon-carbon triple bond and comprising at least 2 up to about 12 carbon atoms.

The term “cycloalkyl,” used alone or as suffix or prefix, refers to a monovalent ring-containing hydrocarbon radical comprising at least 3 up to about 12 carbon atoms.

The term “cycloalkenyl” used alone or as suffix or prefix, refers to a monovalent ring-containing hydrocarbon radical having at least one carbon-carbon double bond and comprising at least 3 up to about 12 carbon atoms.

The term “cycloalkynyl” used alone or as suffix or prefix, refers to a monovalent ring-containing hydrocarbon radical having at least one carbon-carbon triple bond and comprising about 7 up to about 12 carbon atoms.

The term “aryl” used alone or as suffix or prefix, refers to a monovalent hydrocarbon radical having one or more polyunsaturated carbon rings having aromatic character, (e.g., 4n+2 delocalized electrons) and comprising 5 up to about 14 carbon atoms.

The term “arylene” used alone or as suffix or prefix, refers to a divalent hydrocarbon radical having one or more polyunsaturated carbon rings having aromatic character, (e.g., 4n+2 delocalized electrons) and comprising 5 up to about 14 carbon atoms, which serves to links two structures together.

The term “heterocycle” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroalkyl” used alone or as a suffix or prefix, refers to a radical formed as a result of replacing one or more carbon atom of an alkyl with one or more heteroatoms selected from N, O, P and S.

The term “heteroaromatic” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” used alone or as a suffix or prefix, refers to a radical derived from a heterocycle by removing one or more hydrogens therefrom.

The term “heterocyclyl” used alone or as a suffix or prefix, refers a monovalent radical derived from a heterocycle by removing one hydrogen therefrom.

The term “heterocyclylene” used alone or as a suffix or prefix, refers to a divalent radical derived from a heterocycle by removing two hydrogens therefrom, which serves to links two structures together.

The term “heteroaryl” used alone or as a suffix or prefix, refers to a heterocyclyl having aromatic character.

The term “heterocylcoalkyl” used alone or as a suffix or prefix, refers to a heterocyclyl that does not have aromatic character.

The term “heteroarylene” used alone or as a suffix or prefix, refers to a heterocyclylene having aromatic character.

The term “heterocycloalkylene” used alone or as a suffix or prefix, refers to a heterocyclylene that does not have aromatic character.

The term “six-membered” used as prefix refers to a group having a ring that contains six ring atoms.

The term “five-membered” used as prefix refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

The term “substituted” used as a prefix refers to a structure, molecule or group, wherein one or more hydrogens are replaced with one or more C₁₋₁₂hydrocarbon groups, or one or more chemical groups containing one or more heteroatoms selected from N, O, S, F, Cl, Br, I, and P. Exemplary chemical groups containing one or more heteroatoms include heterocyclyl, —NO₂, —OR, —Cl, —Br, —I, —F, —CF₃, —C(═O)R, —C(═O)OH, —NH₂, —SH, —NH, —NR₂, —SR, —SO₃H, —SO₂R, —S(═O)R, —CN, —OH, —C(═O)OR, —C(═O)NR₂, —NRC(═O)R, oxo (═O), imino (═NR), thio (═S), and oximino (═N—OR), wherein each “R” is a C₁₋₁₂hydrocarbyl. For example, substituted phenyl may refer to nitrophenyl, pyridylphenyl, methoxyphenyl, chlorophenyl, aminophenyl, etc., wherein the nitro, pyridyl, methoxy, chloro, and amino groups may replace any suitable hydrogen on the phenyl ring.

The term “substituted” used as a suffix of a first structure, molecule or group, followed by one or more names of chemical groups refers to a second structure, molecule or group, which is a result of replacing one or more hydrogens of the first structure, molecule or group with the one or more named chemical groups. For example, a “phenyl substituted by nitro” refers to nitrophenyl.

The term “optionally substituted” refers to both groups, structures, or molecules that are substituted and those that are not substituted.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazolme, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyt, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolzinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “alkoxy” used alone or as a suffix or prefix, refers to radicals of the general formula —O—R, wherein —R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The term “aryloxy” used alone or as suffix or prefix, refers to radicals of the general formula —O—Ar, wherein —Ar is an aryl.

The term “heteroaryloxy” used alone or as suffix or prefix, refers to radicals of the general formula —O—Ar′, wherein —Ar′ is a heteroaryl.

The term “amine” or “amino” used alone or as a suffix or prefix, refers to radicals of the general formula —NRR′, wherein R and R′ are independently selected from hydrogen or a hydrocarbon radical.

“Acyl” used alone, as a prefix or suffix, means —C(═O)—R, wherein —R is an optionally substituted hydrocarbyl, hydrogen, amino or alkoxy. Acyl groups include, for example, acetyl, propionyl, benzoyl, phenyl acetyl, carboethoxy, and dimethylcarbamoyl.

Halogen includes fluorine, chlorine, bromine and iodine.

“Halogenated,” used as a prefix of a group, means one or more hydrogens on the group is replaced with one or more halogens.

“RT” or “rt” means room temperature.

A first ring group being “fused” with a second ring group means the first ring and the second ring share at least two atoms therebetween.

“Link,” “linked,” or “linking,” unless otherwise specified, means covalently linked or bonded.

### DESCRIPTION OF PREFERRED EMBODIMENTS

In one aspect, the invention provides a compound of formula I, a pharmaceutically acceptable salt thereof, diastereomers, enantiomers, or mixtures thereof: 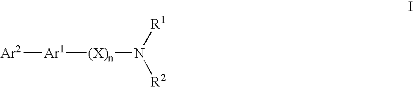
wherein

Ar¹ is arylene, heteroarylene, substituted arylene or substituted heteroarylene, wherein a ring atom of Ar¹ connected to Ar² is seperated from a ring atom of Ar¹ connected to X by at least one atom;

Ar² is aryl, heteroaryl, substituted aryl or substituted heteroaryl;

n is 0 or 1;

X is a divalent group that separates groups connected thereto by one or two atoms;

R¹ is a monovalent C₁₋₂₀ group comprising one or more heteroatoms selected from S, O, N and P;

R² is hydrogen, C₁₋₁₀ alkyl, C₁₋₁₀ acyl, substituted C₁₋₁₀ acyl, substituted C₁₋₁₀ alkyl, C₁₋₁₀ alkylene, or substituted C₁₋₁₀ alkylene, wherein said alkylene is linked to a ring carbon of Ar¹.

Particularly, the compounds of the present invention are those of formula I, wherein

Ar¹ is an arylene, heteroarylene, substituted arylene or substituted heteroarylene, wherein a ring atom of Ar¹ connected to Ar² is seperated from a ring atom of Ar¹ connected to X by at least one atom;

Ar² is an aryl, heteroaryl, substituted aryl or substituted heteroaryl;

X is —CH₂—, or —CH₂—CH₂—;

R² is C₁₋₆ alkyl, substituted C₁₋₆ alkyl, C₁₋₃ alkylene, or substituted C₁₋₃ alkylene, wherein said alkylene is linked to a ring carbon of Ar¹.

More particularly, the compounds of the present invention are those of formula I, wherein

R¹ is selected from:

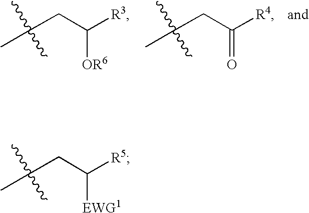

wherein R³ is optionally hydrogen, substituted C₁₋₁₀alkyl, optionally substituted C₅₋₁₂aryl, optionally substituted C₃₋₁₀heteroaryl, optionally substituted aryloxy-C₁₋₆alkyl, optionally substituted heteroaryloxy-C₁₋₆alkyl;

R⁴ and R⁵ are, independently, hydrogen, optionally substituted C₁₋₁₀alkyl, optionally substituted C₅₋₁₂aryl, optionally substituted C₃₋₁₀heteroaryl, amino group, —NHC(═O)—O—R⁷, or —NHC(═O)—R⁷, wherein R⁷ is C₁₋₆alkyl or aryl;

R⁶ is hydrogen, optionally substituted C₁₋₆alkyl, or optionally substituted aryl; and

EWG¹ is an electron withdrawing group.

Even more particularly, the compounds of the present invention are those of formula I, wherein

Ar¹ is optionally substituted para-phenylene, optionally substituted six-membered para-heteroarylene, or optionally substituted monocyclic five-membered meta-heteroarylene;

Ar² is optionally substituted phenyl, or optionally substituted monocylic five or six-membered heteroaryl;

X is —CH₂—, or —CH₂—CH₂—;

R² is C₁₋₃ alkyl, substituted C₁₋₃ alkyl, C₁₋₃ alkylene, or substituted C₁₋₃ alkylene, wherein said alkylene is linked to a ring carbon of Ar¹.

R¹ is selected from:

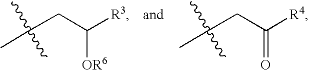

wherein R³ is optionally substituted C₁₋₆alkyl, optionally substituted phenyl, optionally substituted phenoxy-methyl;

R⁴ is, independently, optionally substituted C₁₋₆alkyl, optionally substituted phenyl, amino, —NHC(═O)—O—R⁷, or —NHC(═O)—R⁷, wherein R⁷ is C₁₋₆alkyl or phenyl; and

R⁶ is hydrogen, methyl or ethyl.

Most particularly, the compounds of the present invention are those of formula I, wherein

Ar¹ is para-phenylene or para-pyridylene;

Ar² is a phenyl ortho-substituted with an electron withdrawing group, or a thienyl ortho-substituted with an electron withdrawing group; Even more particularly, Ar² is a phenyl orthosubstituted with —Cl, —F, —OMe, —OEt, —O—CH(CH₃)₂, —CF₃, —NO₂, or —CN; or thienyl ortho-substituted with —Cl, —F, —OMe, —OEt, —O—CH(CH₃)₂, —CF₃, —NO₂, —CN, wherein said ortho-substituted Ar² is optionally further substituted at its non-ortho position;

X is —CH₂—;

R² is methyl.

R¹ is selected from:

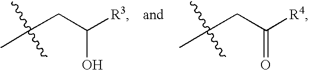

wherein R³ is optionally substituted phenyl, or optionally substituted phenoxy-methyl; Even more particularly, R³ is phenyl, substituted phenoxymethyl or substituted phenyl; and

R⁴is —NHC(═O)—O—R⁷, wherein R⁷ is C₁₋₆alkyl.

In another aspect, the present invention provides a compound of formula II, or a pharmaceutically acceptable salt thereof: 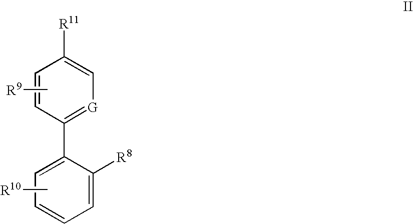
wherein

G is N or CH;

R⁸ is selected from —H, —CH₃, —CF₃, —NO₂ and —CN;

R⁹ is selected from —H and C₁₋₃alkyl;

R¹⁰ is selected from —H and C₁₋₃alkyl; and

R¹¹ is selected from

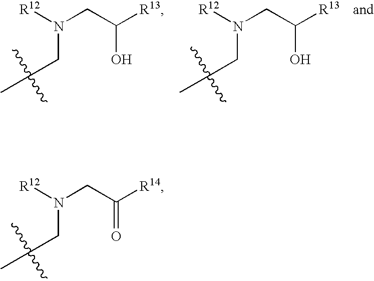

wherein R¹² is H or methyl, R¹³ is phenyl or substituted phenoxymethyl, R¹⁴ is —NHC(═O)OR¹⁵, wherein R¹⁵ is C₁₋₆alkyl.

In a further aspect, the present invention provides a compound of formula III or IV, or a pharmaceutically acceptable salt thereof: 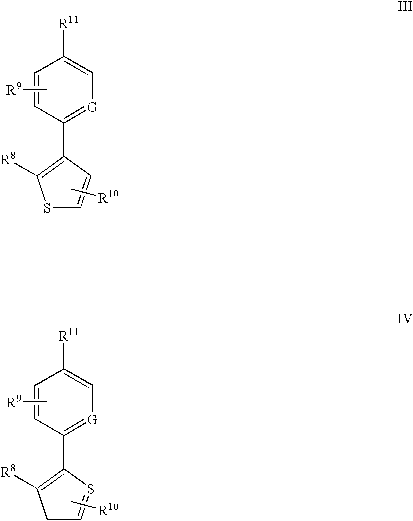
wherein

G is N or CH;

R⁸ is selected from —H, —CH₃, —CF₃, —NO₂ and —CN;

R⁹ is selected from —H and C₁₋₃alkyl;

R¹⁰ is selected from —H and C₁₋₃alkyl; and

R¹¹ is selected from

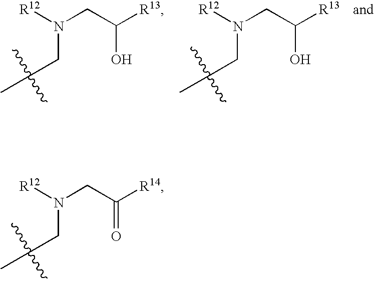

wherein R¹² is H or methyl, R¹³ is phenyl or substituted phenoxymethyl, R¹⁴ is —NHC(═O)OR¹⁵, wherein R¹⁵ is C₁₋₆alkyl.

In an even further aspect, the present invention provides a compound of formula V, or a pharmaceutically acceptable salt thereof: 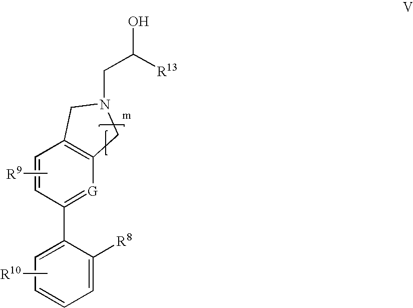
wherein

G is N or CH;

m is 1 or2;

R⁸ is selected from —H, —CH₃, —CF₃, —NO₂ and —CN;

R⁹ is selected from —H and C₁₋₃alkyl;

R¹⁰ is selected from —H and C₁₋₃alkyl; and

R¹³ is phenyl or substituted phenoxymethyl.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I, II, III, IV or V. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formula I, II, III, IV or V. It will further be understood that the present invention encompasses tautomers of the compounds of the formula I, II, III, IV or V.

It will also be understood that certain compounds of the present invention may exist in solvated, for example hydrated, as well as unsolvated forms. It will further be understood that the present invention encompasses all such solvated forms of the compounds of the formula I, II, III, IV or V.

Within the scope of the invention are also salts of the compounds of the formula I, II, III, IV or V. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of formula I, II, III, IV or V above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

We have now found that the compounds of the invention have activity as pharmaceuticals, in particular as modulators or ligands such as agonists, partial agonists, inverse agonist or antagonists of CB₁/CB₂ receptors. More particularly, the compounds of the invention exhibit selective activity as agonist of the CB₁/CB₂ receptors, and are useful in the relief of pain, particularly chronic pain, e.g., chronic inflammatory pain, neuropathic pain, back pain, cancer pain and visceral pain. Compounds of the present invention will also be useful in treating acute pain. Additionally, compounds of the present invention are useful in other disease states in which degeneration or dysfunction of CB₁/CB₂ receptors is present or implicated.

Thus, the invention provides a compound of formula I, II, III, IV or V, or pharmaceutically acceptable salt or solvate thereof, as hereinbefore defined for use in therapy.

In a further aspect, the present invention provides the use of a compound of formula I, II, III, IV or V, or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be contrued accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

The compounds of the present invention are useful in therapy, especially for the therapy of various pain conditions including, but not limited to: acute pain, chronic pain, neuropathic pain, acute pain, back pain, cancer pain, and visceral pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints.

In one embodiment of the invention, the route of administration may be orally, intravenously or intramuscularly.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid and liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or table disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (per cent by weight), more preferably from 0.10 to 50% w, of the compound of the invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the, scope of the invention is the use of any compound of formula I, II, III, IV or V as defined above for the manufacture of a medicament.

Also within the scope of the invention is the use of any compound of formula I, II, III, IV or V for the manufacture of a medicament for the therapy of pain.

Additionally provided is the use of any compound according to Formula I, II, III, IV or V for the manufacture of a medicament for the therapy of various pain conditions including, but not limited to: acute pain, chronic pain, neuropathic pain, acute pain, back pain, cancer pain, and visceral pain.

A further aspect of the invention is a method for therapy of a subject suffering from any of the conditions discussed above, whereby an effective amount of a compound according to the formula I, II, III, IV or V above, is administered to a patient in need of such therapy.

Additionally, there is provided a pharmaceutical composition comprising a compound of Formula I, II, III, IV or V, or a pharmaceutically acceptable salt thereof, in association with a pharmaceutically acceptable carrier.

Particularly, there is provided a pharmaceutical composition comprising a compound of Formula I, II, III, IV or V, or a pharmaceutically acceptable salt thereof, in association with a pharmaceutically acceptable carrier for therapy, more particularly for therapy of pain.

Further, there is provided a pharmaceutical composition comprising a compound of Formula I, II, III, IV or V, or a pharmaceutically acceptable salt thereof, in association with a pharmaceutically acceptable carrier use in any of the conditions discussed above.

In a further aspect, the present invention provides a method of preparing a compound of the present invention using one or more of the general procedures below, wherein Rₐ and R_{b} are independently selected from —H, optionally substituted C₁₋₆alkyl, optionally substituted aryl, optionally substituted heteroaryl, —CF₃, —NO₂, and —CN; n is 1 or 2; R_{c}, R_{d}, Rₑ and R_{f} are independently selected from —H, C₁₋₃alkyl,

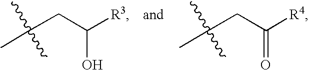

wherein R³ is optionally substituted phenyl, or optionally substituted phenoxy-methyl;

R⁴ is —NHC(═O)—O—R⁷, wherein R⁷ is C₁₋₆alkyl; R_{c1} is —H or C₁₋₃alkyl; and R_{g} is optionally subsitituted phenyl or optionally subsituted phenoxymethyl. 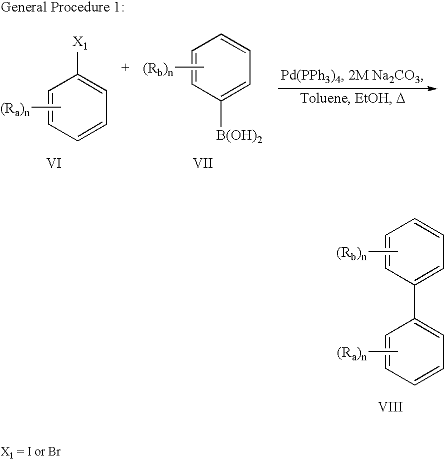
A solution of the aryl boronic acid (VII, 1.5 equiv.) in ethanol (3 mL/mmol boronic acid) was added to a mixture of the aryl halide (VI, 1 equiv.), Pd(PPh₃)₄ (0.05 equiv.), toluene (9 mL/mmol aryl halide), and 2 M Na₂CO₃ (6.7 equiv.). The resulting mixture was heated at reflux until the aryl halide was consumed (typically 16 h). The reaction was then concentrated in vacuo, and the residue was diluted with water. The aqueous phase was extracted with EtOAc (3×). The combined organic phases were then washed with brine, dried over MgSO₄, filtered through Celite, and concentrated in vacuo. The residue was dissolved in methanol and allowed to stand overnight. The orange solid which precipitated was filtered, and the supernatant was concentrated in vacuo to provide the title compound. The product (VIII) was used for subsequent steps, or purified by silica gel column chromatography when necessary.

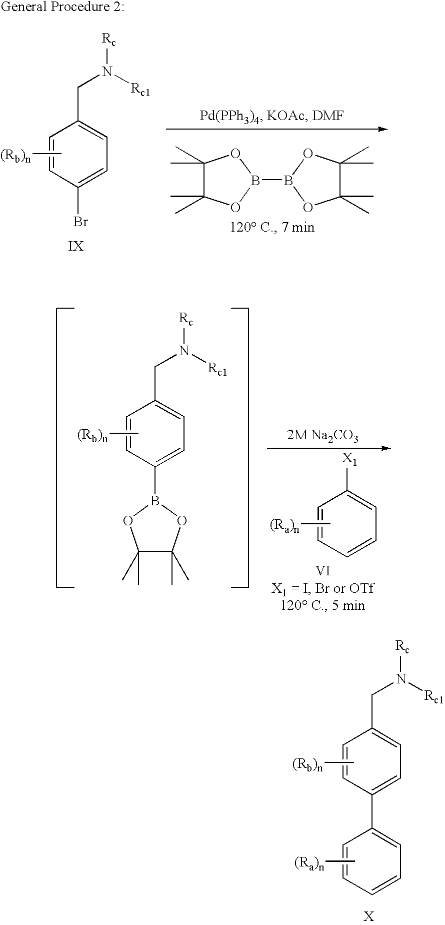

Solutions of the aryl bromide (IX, 1 equiv.) in DMF (3 mL/mmol aryl bromide) and bis(pinacolato)diboron (1.1 equiv.) in DMF (2.7 mL/mmol diboron compound) were added successively to a mixture of Pd(PPh₃)₄ (0.03 equiv.) and KOAc (3 equiv.) contained in a microwave process vial. The vial was capped and heated to 120° C. for 7 min using microwave irradiation. The resulting mixture was cooled, and 2 M Na₂CO₃ (4.9 equiv.) and a solution of the second aryl halide or aryl triflate (VI, 1-2 equiv.) in DMF (0.3-0.9 mL/mmol aryl halide/triflate, depending on solubility) were added to the vial through the septum cap. The reaction was heated to 120° C. for an additional 5 minutes using microwave irradiation. The resulting mixture was diluted with water (6 mL/mmol of initial aryl halide used) and CH₂Cl₂ (24 mL/mmol of initial aryl halide used), loaded onto an Extube® Chem Elut column (Varian), and eluted with two column volumes of CH₂Cl₂. The eluant was concentrated, and the residue was dissolved in CH₂Cl₂ (12 mL/mmol of initial aryl halide used). MP-TsOH resin was added to the solution, and the mixture was stirred for 2 hours. The resin was removed by filtration and washed with additional CH₂Cl₂ and MeOH. The filtrate and washings were discarded, and the compound (X) was then released from the resin using 2M NH₃ in MeOH. The release solution was concentrated to provide the compound (X).

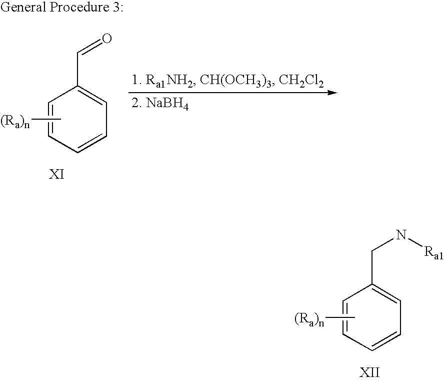

A solution of Rₐ₁NH₂ in MeOH (2 M, 5 equiv.) was added to a mixture of the aldehyde (XI, 1 equiv.) and CH(OCH₃)₃ (10 equiv) in CH₂Cl₂ (7.5 mL/mmol aldehyde). The resulting mixture was stirred overnight at room temperature, and then NaBH₄ (2.5 equiv.) was added. When the starting aldehyde/intermediate imine had been completely consumed, the reaction was concentrated in vacuo. The residue was taken into EtOAc (10 mL/mmol aldehyde used) and the product was extracted into 1 N HCl (3×7.5 mL/mmol aldehyde used). The EtOAc layer was discarded, the combined aqueous layers were basicified with 6 N NaOH, and the product was back extracted with EtOAc (3×10 mL/mmol aldehyde used). The combined organic phases were then dried over Na₂SO₄, filtered, and concentrated in vacuo to provide the compound (XI). The compound (XII) was used for subsequent steps, or purified by silica gel column chromatography when necessary.

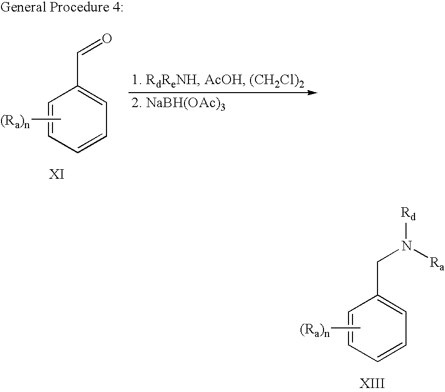

A solution of the amine (R_{d}RₑNH, 1 equiv.) and aldehyde (XI, 1-2 equiv.) in AcOH/dichloroethane (5% v/v, 10 ml/mmol amine) was stirred at room temperature overnight. NaBH(OAc)₃ (2 equiv.) was then added. When the starting aldehyde/intermediate imine/iminium ion had been completely consumed, saturated Na₂CO₃ (6 mL/mmol amine) was added. The layers were separated, and the aqueous layer was extracted with additional EtOAc (3×12 mL/mmol amine). The combined organic phases were then dried over Na₂SO₄, filtered, and concentrated in vacuo to provide the compound (XIII). The compound (XIII) was used for subsequent steps, or purified by silica gel column chromatography or reverse phase HPLC when necessary.

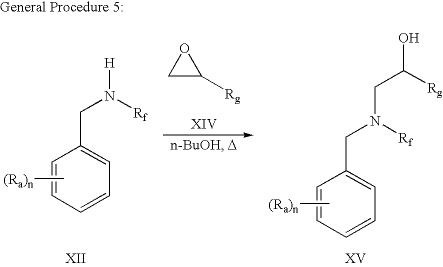

A solution of the amine (XII, 1 equiv.) and epoxide (XIV, 1 equiv.) in n-BuOH (6 mL/mmol amine) was heated at the temperature specified until the starting materials were consumed. The reaction was concentrated in vacuo, and the residue was purified by reverse phase HPLC to provide the compound (XV).

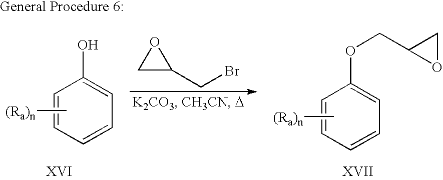

A suspension of the phenol (XVI, 1 equiv.), epibromohydrin (5 equiv.), and K₂CO₃ (5 equiv.) in dry CH₃CN (8 mL/mmol phenol) was heated at 70° C. until the starting phenol was completely consumed (typically 16 h). The reaction mixture was filtered to remove solids which were then washed with additional CH₃CN. The filtrate was concentrated to provide the compound (XVII).

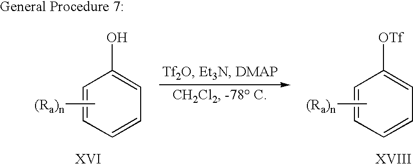

Triethylamine (2.2 equiv.), followed by triflic anhydride (1.1 equiv.), was added dropwise to a solution of the phenol (XVI, 1 equiv.) and DMAP (0.1 equiv.) in dry CH₂Cl₂ (10 mL/mmol phenol) maintained at −78° C. The reaction was allowed to slowly warm to room temperature and stirred until the starting phenol was completely consumed (typically 16 h). Once the reaction was complete, water was added (10 mL/mmol phenol), the layers were separated, and the aqueous phase was extracted with CH₂Cl₂ (2×10 mL/mmol phenol). The combined organic phases were then dried over Na₂SO₄, filtered, and concentrated in vacuo. Silica gel column chromatography on the organic phase residue provided the compound (XVIII).

The compounds of the invention were found to be active towards CB₁/CB₂ receptors in warm-blooded animal, e.g., human. Particularly the compounds of the invention have been found to be effective CB₁/CB₂ receptor agonists. In vitro assays, infra, demonstrated these surprising activities. In these in vitro assays, a compound is tested for their activity toward CB₁/CB₂ receptors and the dissociation constant (Ki) is obtained to determine the selective activity for a particular compound towards CB₁/CB₂ receptors by measuring IC₅₀ of the compound. In the current context, IC₅₀ generally refers to the concentration of the compound at which 50% displacement of a standard radioactive CB₁/CB₂ receptor ligand has been observed. Generally, a lower Ki for a particular compound towards CB₁/CB₂ receptors means that the particular compound is a stronger ligand towards the CB₁/CB₂ receptors. As a result, compounds with relatively low Ki towards CB₁/CB₂ receptors are relatively strong CB₁/CB₂ receptor ligands or strong CB₁/CB₂ receptor agonists.

Biological Evaluation

hCB₁ and hCB₂ Receptor Binding

Human CB₁ receptor from Receptor Biology (hCB1) or human CB₂ receptor from BioSignal (hCB2) membranes are thawed at 37° C., passed 3 times through a 25-gauge blunt-end needle, diluted in the cannabinoid binding buffer (50 mM Tris, 2.5 mM EDTA, 5 mM MgCl₂, and 0.5 mg/mL BSA fatty acid free, pH 7.4) and aliquots containing the appropriate amount of protein are distributed in 96-well plates. The IC₅₀ of the compounds of the invention at hCB₁ and hCB₂ are evaluated from 10-point dose-response curves done with ³H-CP55,940 at 20000 to 25000 dpm per well (0.17-0.21 nM) in a final volume of 300 μl. The total and non-specific binding are determined in the absence and presence of 0.2 FM of HU210 respectively. The plates are vortexed and incubated for 60 minutes at room temperature, filtered through Unifilters GF/B (presoaked in 0.1% polyethyleneimine) with the Tomtec or Packard harvester using 3 mL of wash buffer (50 mM Tris, 5 mM MgCl₂, 0.5 mg BSA pH 7.0). The filters are dried for 1 hour at 55° C. The radioactivity (cpm) is counted in a TopCount (Packard) after adding 65 μl/well of MS-20 scintillation liquid.

Based on the above assays, the dissociation constant (Ki) for a particular compound of the invention towards a particular receptor is determined using the following equation:
Ki=IC₅₀/(1+[rad]/Kd),

Wherein IC₅₀ is the concentration of the compound of the invention at which 50% displacement has been observed;

[rad] is a standard or reference radioactive ligand concentration at that moment; and

Kd is the dissociation constant of the radioactive ligand towards the particular receptor.

Biological data for certain compounds of the invention are listed in Table 1 below.

TABLE 1

| Compound | CB₂ | CB₁ |
|---|---|---|
| No. | (Ki, nM) | (Ki, nM) |
| 1-132 | 15-2800 | 50-5000 |

### EXAMPLES

The invention will further be described in more detail by the following Examples which describe methods whereby compounds of the present invention may be prepared, purified, analyzed and biologically tested, and which are not to be construed as limiting the invention.

### Example 1

### α-[[Methyl[(2′-methyl[1,1′-biphenyl]-4-yl)methyl]amino]methyl]-benzenemethanol

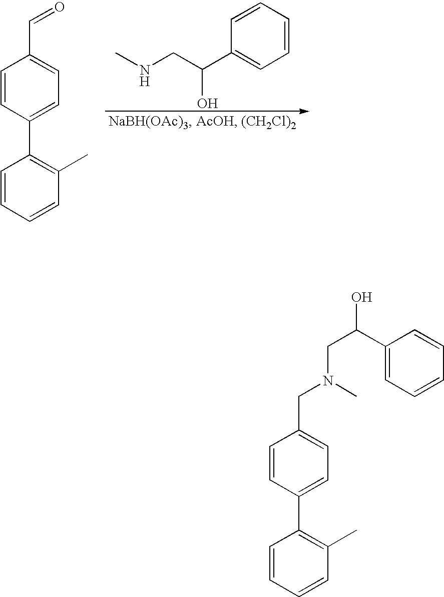

Following General Procedure 4, 2′-methyl-[1,1′-biphenyl]-4-carboxaldehyde (0.250 g, 1.28 mmol), α-[(methylamino)methyl]benzenemethanol (0.363 g, 2.40 mmol), and NaBH(OAc)₃ (0.506 g, 2.40 mmol) were combined. When the starting imine intermediate had been completely consumed, 1 N NaOH (10 mL/mmol amine) was added. The layers were then filtered through a Hydromatrix®column and the product was eluted with CH₂Cl₂. The organic phase was concentrated in vacuo and purified by reverse phase HPLC (gradient 20-100% CH₃CN in H₂O) to provide the title compound (0.052 g, 11%) as its HCO₂H salt. Due to quatemization of the stereogenic nitrogen atom, a mixture of 2 diastereomeric salts was obtained. ¹H-NMR (CDCl₃): δ 7.39-7.23 (br m, 13H), 4.83 (dd, J=3.8 Hz, J=10.2 Hz, 1H), 3.94-3.85 (overlapping br s at 3.94 and d at 3.87, J=13.2 Hz, 2H), 3.68 (d, J=12.8 Hz, 1H), 2.72 (dd, J=10.0 Hz, J=12.4 Hz, 1H), 2.63 (dd, J=3.6 Hz, J=12.0 Hz, 1H), 2.44 (s, 3H), 2.28 (s, 3H). MS (ESI) (M+H)⁺=332. Anal. Calcd for C₂₃H₂₅NO+0.30 CH₂O₂: C, 81.06; H, 7.47; N, 4.06. Found: C, 81.40; H, 7.76; N, 4.18.

### Example 2

### α-[[[(2′-Methoxy[1,1′-biphenyl]-4-yl)methyl]methylamino]methyl]-benzenemethanol

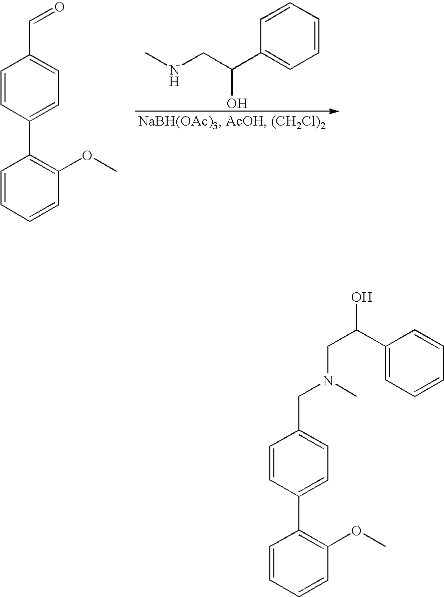

Following General Procedure 4, 2′-methoxy-[1,1′-biphenyl]-4-carboxaldehyde (0.250 g, 1.18 mmol), α-[(methylamino)methyl]benzenemethanol (0.363 g, 2.40 mmol), and NaBH(OAc)₃ (0.506 g, 2.40 mmol) were combined. When the starting imine intermediate had been completely consumed, 1 N NaOH (10 mL/mmol amine) was added. The layers were then filtered through a Hydromatrix® column and the product was eluted with CH₂Cl₂. The organic phase was concentrated in vacuo and purified by reverse phase HPLC (gradient 20-100% CH₃CN in H₂O) to provide the title compound (0.048 g, 10%) as its HCO₂H salt. Due to quaternization of the stereogenic nitrogen atom, a mixture of 2 diastereomeric salts was obtained. ¹H-NMR (CDCl₃): δ 7.54 (d, J=8.4 Hz, 2H), 7.40-7.25 (br m, 9H), 7.05-6.98 (m, 2H), 4.88 (dd, J=2.6 Hz, J=10.2 Hz, 1H), 4.55 (br s, 1H), 3.91 (d, J=13.6 Hz, 1H), 3.81-3.74 (overlapping s at 3.81 and d at 3.75, J=13.2 Hz, 4H), 2.79 (dd, J=10.0 Hz, J=13.2 Hz, 1H), 2.68 (dd, J=3.2 Hz, J=12.8 Hz, 1H), 2.48 (s, 3H). MS (ESI) (M+H)⁺=348. Anal. Calcd for C₂₃H₂₅NO₂+0.40 CH₂O₂: C, 76.82; H, 7.11; N, 3.83. Found: C, 76.98; H, 7.17; N, 3.77.

### Example 3

### α-[[[(2′-Chloro[1,1′-biphenyl]-4-yl)methyl]methylamino]methyl]-benzenemethanol

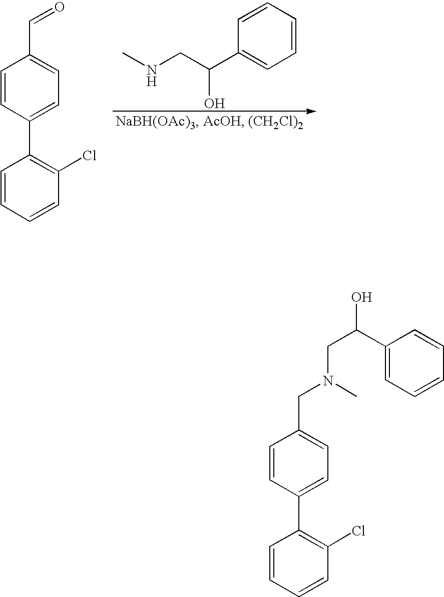

Following General Procedure 4, 2′-chloro-[1,1′-biphenyl]-4-carboxaldehyde (0.250 g, 1.16 mmol), α-[(methylamino)methyl]benzenemethanol (0.363 g, 2.40 mmol), and NaBH(OAc)₃ (0.506 g, 2.40 mmol) were combined. When the starting imine intermediate had been completely consumed, 1 N NaOH (10 mL/mmol amine) was added. The layers were then filtered through a Hydromatrix® column and the product was eluted with CH₂Cl₂. The organic phase was concentrated in vacuo and purified by reverse phase HPLC (gradient 20-100% CH₃CN in H₂O) to provide the title compound (0.050 g, 11%) as its HCO₂H salt. Due to quaternization of the stereogenic nitrogen atom, a mixture of 2 diastereomeric salts was obtained. ¹H-NMR (CDCl₃): δ 7.49-7.26 (br m, 13H), 4.85 (dd, J=3.2 Hz, J=10.8 Hz, 1H), 4.18 (br s, 1H), 3.89 (d, J=12.8 Hz, 1H), 3.72 (d, J=13.2 Hz, 1H), 2.75 (dd, J=10.4 Hz, J=12.8 Hz, 1H), 2.65 (dd, J=3.2 Hz, J=12.8 Hz, 1H), 2.46 (s, 3H). MS (ESI) (M+H)⁺=352. Anal. Calcd for C₂₂H₂₂NOCl+0.30 CH₂O₂: C, 73.25; H, 6.23; N, 3.83. Found: C, 73.44; H, 6.31; N, 3.86.

### Example 4

### α-[[Methyl-[[2′-(trifluoromethyl)-[1,1′-biphenyl]-4-yl]methyl]amino]methyl]-benzenemethanol

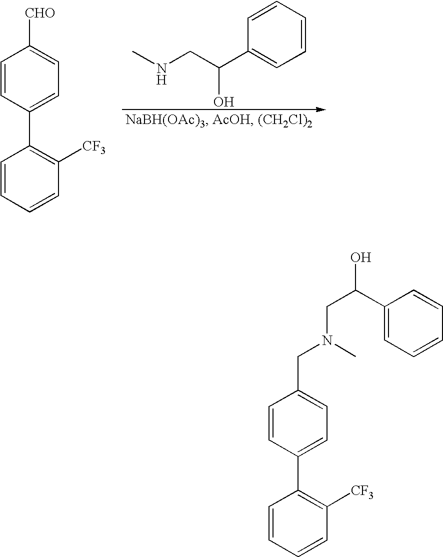

Following General Procedure 4, 2′-(trifluoromethyl)-[1,1′-biphenyl]-4-carboxaldehyde (0.500 g, 2.00 mmol), α-[(methylamino)methyl]benzenemethanol (0.604 g, 4.00 mmol), and NaBH(OAc)₃ (0.844 g, 4.00 mmol) were combined. The crude product was purified by flash chromatography (3:7 Hexanes:EtOAc) to provide the title compound. HCl in Et₂O (2 mL of 1M, 2.00 mmol) was added to the compound and the resulting solid was filtered and washed with additional Et₂O to provide the HCl salt (0.558 g, 66%). Due to quaternization of the stereogenic nitrogen atom, a mixture of 2 diastereomeric salts was obtained. ¹H-NMR (CD₃OD): δ 7.81 (d, J=7.6 Hz, 1H), 7.71-7.56 (m, 4H), 7.52-7.32 (m, 8H), 5.15-5.09 (m, 1H), 4.77 (br d, J=14.0 Hz, 0.5H), 4.50 (AB_{q}, 1H), 4.33 (br d, J=12.0 Hz, 0.5H), 3.46-3.15 (m, 2H), 3.08 (s, 1.5H), 2.92 (s, 1.5H). MS (ESI) (M+H)⁺=386. Anal. Calcd for C₂₃H₂₂F₃NO+1.1 HCl: C, 64.92; H, 5.47; N, 3.29. Found: C, 65.16; H, 5.63; N, 3.37.

### Example 5

### 1-(3,4-Dichlorophenoxy)-3-[methyl[[2′-(trifluoromethyl)[1,1′-biphenyl]-4yl]methyl]amino]-2-propanol

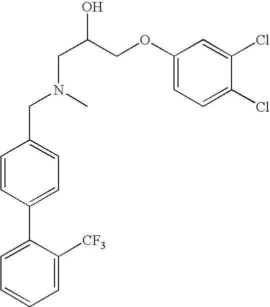

### Compound 5A: N-Methyl-2′-(trifluoromethyl)-[1,1′-biphenyl]-4-methanamine

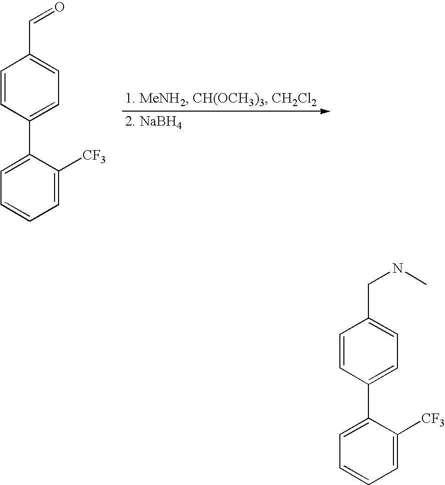

Following General Procedure 3, 2′-(trifluoromethyl)-[1,1′-biphenyl]-4-carboxaldehyde (0:400 g, 1.60 mmol) was converted to the title compound (0.297 g, 70%). The crude material was of sufficient purity (>90%) to be used in subsequent steps. ¹H-NMR (CDCl₃) δ 7.75 (d, J=7.6 Hz, 1H), 7.56 (t, J=7.2 Hz, 1H), 7.46 (d, J=7.6 Hz, 1H), 7.42-7.28 (m, 5H), 3.82 (s, 2H), 2.51 (s, 3H), 2.13 (br s, 1H). MS (ESI) (M+H)⁺=266.

### Compound 5b: 1-(3,4-Dichlorophenoxy)-3-[methyl[[2′-(trifluoromethyl)[1,1′-biphenyl]-4-yl]methyl]amino]-2-propanol

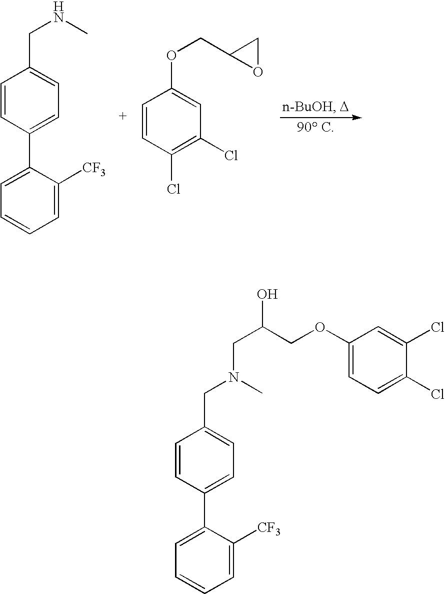

Following General Procedure 5, N-methyl-2′-(trifluoromethyl)-[1,1′-biphenyl]-4-methanamine (0.133 g, 0.40 mmol) and 2-[(3,4-dichlorophenoxy)methyl]oxirane (0.088 g, 0.40 mmol) were combined and heated at 50° C. for 24 h. The crude product was purified by reverse phase HPLC (gradient 30-70% CH₃CN in H₂O) to provide the title compound (0.026 g, 11%) as its TFA salt. This material was lyophilized from H₂O/CH₃CN to produce a white solid. ¹H-NMR (CDCl₃): δ 7.77 (d, J=7.6 Hz, 1H), 7.60 (t, J=7.4 Hz, 1H), 7.53-7.51 (m, 3H), 7.43 (d, J=8.0 Hz, 2H), 7.34-7.31 (overlapping s at 7.33 and d at 7.32, J=8.8 Hz, 2H), 6.97 (d, J=2.8 Hz, 1H), 6.73 (dd, J=2.8 Hz, J=8.8 Hz, 1H), 4.50 (br s, 1H), 4.36 (br s, 2H), 4.07 (br s, 1H), 3.89 (t, J=8.2 Hz, 1H), 3.51-3.03 (br s at 3.36 and br s at 3.16, 2H), 2.94 (br s, 3H). MS (ESI) (M+H)⁺=484. Anal. Calcd for C₂₄H₂₂Cl₂F₃NO₂+0.3 H₂O+0.9 TFA: C, 52.31; H, 4.00; N, 2.36. Found: C, 52.32; H, 3.93; N, 2.24.

### Example 6

### α-[(2-Fluoro-4-nitrophenoxy)methyl]-3,4-dihydro-6-[2-(trifluoromethyl)phenyl]-2(1H)-isoquinolineethanol

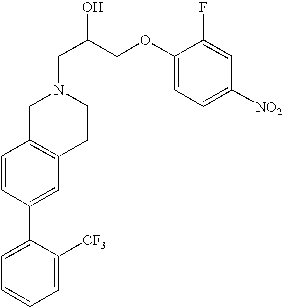

### Compound 6a: 2-[(2-Fluoro-4-nitrophenoxy)methyl]oxirane

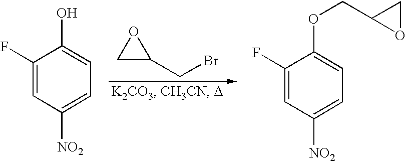

Following General Procedure 6, 2-fluoro-4-nitrophenol (0.471 g, 3.00 mmol) was converted to the title compound (0.635 g, 99%). The crude compound was used for subsequent steps. ¹H-NMR (CDCl₃): δ 8.06 (ddd, J=1.2 Hz, J=2.4 Hz, J=8.8 Hz, 1H), 8.00 (dd, J=2.4 Hz, J=10.4 Hz, 1H), 7.10 (dd, J=8.0 Hz, J=9.2 Hz, 1H), 4.48 (dd, J=2.4 Hz, J=11.2 Hz, 1H), 4.11 (dd, J=6.0 Hz, J=11.6 Hz, 1H), 3.45-3.39 (m, 1H), 2.97 (dd, J=4.0 Hz, J=4.8 Hz, 1H), 2.81 (dd, J=2.8 Hz, J=4.8 Hz, 1H).

### Compound 6b: 3-Bromobenzeneethanamine

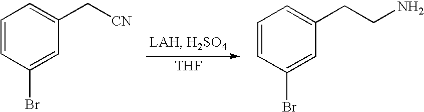

A suspension of LiAlH₄ (1.24 g, 32.7 mmol) in dry THF (50 mL) was cooled to 0° C. Concentrated H₂SO₄ (1.6 g, 16.3 mmol) was added dropwise, and the resulting mixture was stirred at 0° C. for 30 min. A solution of 3-bromo-benzeneacetonitrile (4.01 g, 20.4 mmol) in THF (5 mL) was added dropwise, and the reaction was allowed to warm to room temperature when the addition was complete. The reaction was stirred at room temperature for 1 h, and then cooled back to 0° C. and quenched by the addition of a 1:1 THF:H₂O mixture (5 mL). Et₂O was added (20 mL), followed by a 3.6 M solution of NaOH (10 mL). The mixture was filtered through Celite, and the solids were washed well with additional Et₂O. The organic phase was dried over Na₂SO₄, filtered, and concentrated in vacuo to provide the title compound (3.91 g, 96%). The crude compound was used in subsequent steps., ¹H-NMR (CDCl₃): δ 7.38-7.30 (overlapping s at 7.35 and d, J=7.2 Hz for d, 2H), 7.20-7.10 (m, 2H), 2.96 (t, J=6.8 Hz, 2H), 2.72 (t, J=6.4 Hz, 2H), 1.35 (br s, 2H) MS (ESI) (M+H)⁺=200/202.

### Compound 6c: N-[2-(3-Bromophenyl)ethyl]-2,2,2-trifluoroacetamide

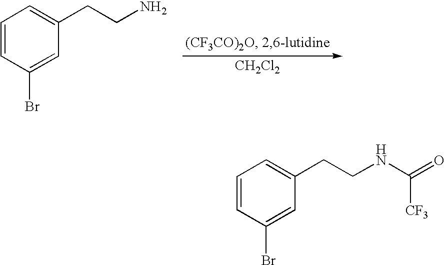

A mixture of 3-bromobenzeneethanamine (2.00 g, 10.0 mmol) and 2,6-lutidine (1.2 mL, 10.3 mmol) in dry CH₂Cl₂ (40 mL) was cooled to 0° C. Trifluoroacetic anhydride (1.4 mL, 9.9 mmol) was added dropwise, and the reaction was then warmed to room temperature and allowed to stir for 16 h. Water (40 mL) was added to the reaction, the phases were separated, and the aqueous layer was extracted with CH₂Cl₂ (2×40 mL). The combined organic phases were washed successively with 1 M HCl (40 mL) and saturated NaHCO₃ (40 mL), and then dried over Na₂SO₄, filtered, and concentrated in vacuo to provide the title compound (2.93 g, 100%). The crude compound was used in subsequent steps. ¹H-NMR (CDCl₃): δ 7.40 (d, J=8.0 Hz, 1H), 7.36 (s, 1H), 7.21 (t, J=7.6 Hz, 1H), 7.12 (d, J=7.6 Hz, 1H), 6.33 (br s, 1H), 3.59 (q, J=6.8 Hz, 2H), 2.87 (t, J=7.2 Hz, 2H). MS (ESI) (M+H)⁺=296/298.

### Compound 6d: 6-Bromo-1,2,3,4-tetrahydro-2-(trinuoroacetyl)isoquinoline and 8-bromo-1,2,3,4tetrahydro-2trifluoroacetyl)isoquinoline

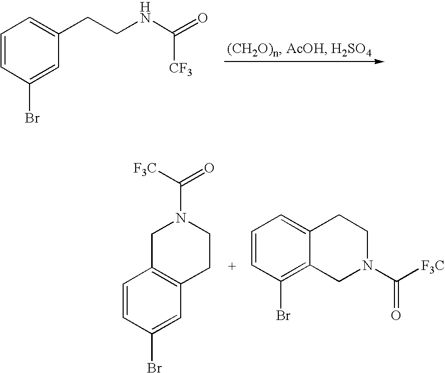

A mixture of glacial acetic acid (22.5 mL) and concentrated sulfuric acid (15 mL) was added to a mixture of N-[2-(3-bromophenyl)ethyl]-2,2,2-trifluoroacetamide (4.06 g, 13.7 mmol) and paraformaldehyde (0.659 g, 22.0 mmol equiv. of formaldehyde). The reaction was stirred at room temperature for 16 h, and then poured into 300 mL of cold water. The aqueous solution was extracted with EtOAc (3×100 mL). The combined organic phases were washed with saturated NaHCO₃ (75 mL) and water (2×150 mL). The organic phase was then dried over Na₂SO₄, filtered, and concentrated in vacuo. The residue was purified by column chromatography (4:1 Hexanes:EtOAc) to provide a mixture of the title compounds (3.31 g, 78%). Due to hindered rotation about the amide bond, rotamers were observed in the ¹H-NMR spectrum. ¹H-NMR (CDCl₃): δ 7.46 (dd, J=2.0 Hz, J=6.8 Hz, 0.33H, 7.38-7.31 (m, 1.33H), 7.15-7.09 (m, 0.67H), 7.05-6.98 (m, 0.67H), 4.75, 4.73, 4.69 (3×s, 2H), 3.90-3.80 (m, 2H), 3.00-2.90 (m, 2H). MS ESI) (M+H)⁺=308/310.

### Compound 6e: 1,2,3,4-Tetrahydro-6-[2-(trifluoromethyl)phenyl]isoquinoline and 1,2,3,4-tetrahydro-8-[2-(trifluoromethyl)phenyl]isoquinoline

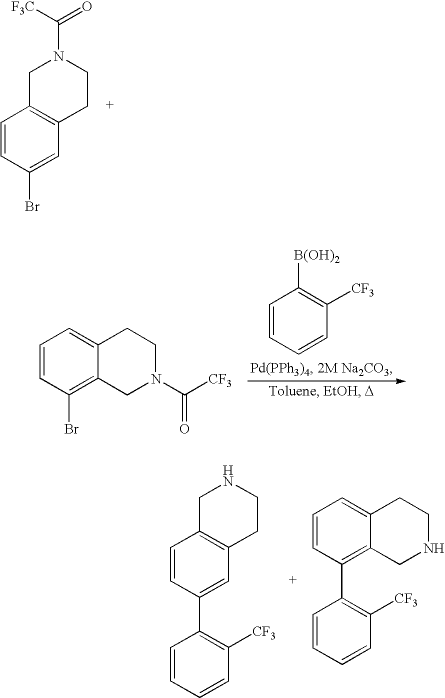

Following General Procedure 1, a mixture of 6-bromo-1,2,3,4-tetrahydro-2-(trifluoroacetyl)isoquinoline and 8-bromo-1,2,3,4-tetrahydro-2-(trifluoroacetyl)-isoquinoline (0.137 g, 0.446 mmol) was reacted with [2-(trifluoromethyl)phenyl]-boronic acid (0.127 g, 0.668 mmol) to provide a mixture of the title compounds. Purification by column chromatography (4:1 CH₂Cl₂:MeOH+0.1% conc. NH₃) provided 1,2,3,4tetrahydro-8-[2-(trifluoromethyl)phenyl]isoquinoline (0.0380 g, 31%) and 1,2,3,4-tetrahydro-6-[2-(trifluoromethyl)phenyl]isoquinoline (0.0810 g, 65%).

1,2,3,4-tetrahydro-8-[2-(trifluoromethyl)phenyl]isoquinoline: ¹H-NMR (CDCl₃): δ 7.77 (d, J=7.2 Hz, 1H), 7.56 (t, J=7.6 Hz, 1H), 7.49 (t, J=7.6 Hz, 1H), 7.23 (d, J=7.6 Hz, 1H), 7.21 (t, J=7.6 Hz, 1H), 7.16 (d, J=6.8 Hz, 1H), 7.01 (d, J=7.6 Hz, 1H), 4.66 (br s, 1H), 3.72 (half of br AB_{q}, J=16.0 Hz, 1H), 3.57 (alf of br AB_{q}, J=15.6 Hz, 1H), 3.19 (br s, 2H), 2.97 (br s, 2H). MS (ESI) (M+H)⁺=278. 1,2,3,4-tetrahydro-6-[2-(trifluoromethyl)phenyl]isoquinoline: ¹H-NMR (CDCl₃): δ 7.74 (d, J=7.6 Hz, 1H), 7.55 (t, J=6.8 Hz, 1H), 7.45 (t, J=8.0 Hz, 1H), 7.31 (d, J=7.6 Hz, 1H), 7.12 (d, J=8.4 Hz, 1H), 7.07 (s, 1H), 7.06 (d, J=8.0 Hz, 1H), 4.12 (br s, 2H), 3.87 (br s, 1H), 3.23 (br s, 2H), 2.88 (br s, 2H). MS (ESI) (M+H)⁺=278.

### Compound 6f: a-[(2-Fluoro-4-nitrophenoxy)methyl]-3,4-dihydro-6-[2-(trifluoromethyl)phenyl]-2(1H)-isoquinolineethanol

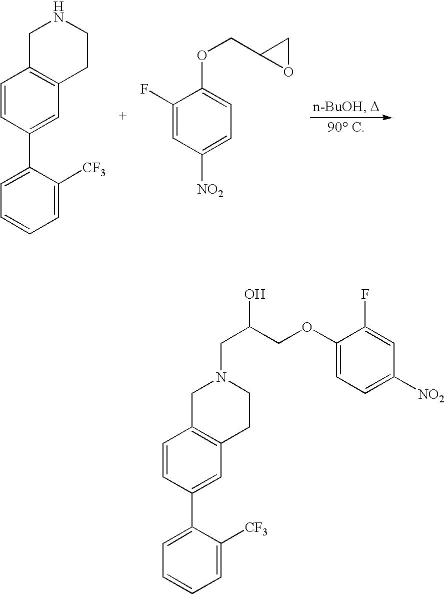

Following General Procedure 5, 1,2,3,4-tetrahydro-6-[2-(trifluoromethyl)phenyl]-isoquinoline (0.0256 g, 0.0923 mmol) and 2-[(2-fluoro-4-nitrophenoxy)methyl]-oxirane (0.0197 g, 0.0924 mmol) were combined and heated at 90° C. for 16 h. The crude product was purified by reverse phase HPLC (gradient 20-60% CH₃CN in H₂O) to provide the title compound (0.0222 g, 40%) as its TFA salt. This material was lyophilized from H₂O/acetonitrile to produce a white, hygroscopic solid. Due to quaternization of the stereogenic nitrogen atom, a mixture of 2 diastereomeric salts was obtained. ¹H-NMR (CD₃OD): δ 8.15-8.11 (m, 1H), 8.08 (dd, J=2.8 Hz, J=11.2 Hz, 1H), 7.79 (d, J=8.0 Hz, 1H), 7.66 (t, J=7.6 Hz, 1H), 7.57 (t, J=7.6 Hz, 1H), 7.39-7.24 (m, 5H), 4.82-4.50 (br m, 3H), 4.29 (d, J=4.8 Hz, 2H), 3.95 (br s, 1H), 3.62-3.52 (m, 3H), 3.38-3.22 (br m, 2H). MS (ESI) (M+H)⁺=491. Anal. Calcd for C₂₅H₂₂F₄N₂O₄+1.1 TFA+0.7 H₂O: C, 51.98; H, 3.93; N, 4.46. Found: C, 52.02; H, 3.93; N, 4.42.

### Example 7: Ethyl [[methyl-[[2′-(trifluoromethyl)-[1,1′-biphenyl]-4-yl]methyl]amino]-acetyl]carbamate

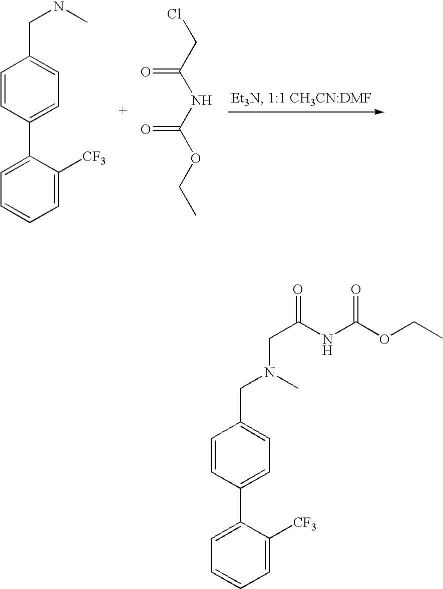

A mixture of N-methyl-2′-(trifluoromethyl)-[1,1′-biphenyl]-4-methanamine (0.0781 g, 0.294 mmol), ethyl N-(chloroacetyl)carbamate (0.0487 g, 0.294 mmol), and triethylamine (0.041 mL, 0.29 mmol) in 1:1 CH₃CN:DMF (3 mL) was stirred at room temperature for 24 h. The reaction mixture was concentrated, and the residue was partitioned between CH₂Cl₂ (5 mL) and H₂O (5 mL). The phases were separated, and the aqueous phase was extracted with CH₂Cl₂ (3×5 mL). The combined organic phases were dried over Na₂SO₄, filtered, and concentrated in vacuo. The crude product was purified by reverse phase HPLC (gradient 20-60% CH₃CN in H₂O) to provide the title compound (0.0992 g, 86%) as its TFA salt. This material was lyophilized from H₂O/acetonitrile. ¹H-NMR (CD₃OD): δ 7.81 (d, J=8.0 Hz, 1H), 7.68 (t, J=7.6 Hz, 1H), 7.63 (d, J=8.0 Hz, 2H), 7.59 (t, J=8.0 Hz, 1H), 7.46 (d, J=8.0 Hz, 2H), 7.38 (d, J=7.6 Hz, ¹H), 4.70-4.30 (br, 3H), 4.24 (q, J=7.2 Hz, 2H), 2.95 (s, 3H), 1.31 (t, J=7.2 Hz, 3H). MS (ESI) (M+H)⁺=395. Anal. Calcd for C₂₀H₂₁F₃N₂O₃+1.3 TFA+0.4 H₂O: C, 49.37; H, 4.23; N, 5.09. Found: C, 49.45; H, 4.23; N, 5.05.

### Example 8

### 3,4-Dihydro-α-phenyl-7-[2-(trifluoromethyl)phenyl]-2(1H)-isoqulolineethanol

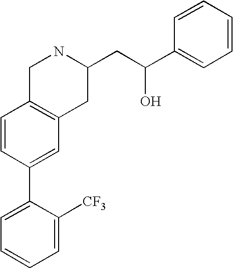

### Compound 8a: N-[2-(4Bromophenyl)ethyl]-2,2,2-trifluoroacetamide

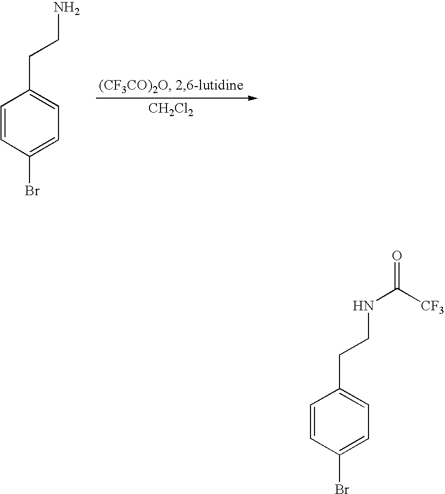

A mixture of 4-bromobenzeneethanamine (1.23 g, 6.17 mmol) and 2,6-lutidine (0.76 mL, 6.5 mmol) in dry CH₂Cl₂ (25 mL) was cooled to 0° C. Trifluoroacetic anhydride (0.87 mL, 6.2 mmol) was added dropwise, and the reaction was then warmed to room temperature and allowed to stir for 16 h. Water (25 mL) was added to the reaction, the phases were separated, and the aqueous layer was extracted with CH₂Cl₂ (2×25 mL). The combined organic phases were washed successively with 1 M HCl (25 mL) and saturated NaHCO₃ (25 mL), and then dried over Na₂SO₄, filtered, and concentrated in vacuo to provide the title compound (1.79 g, 98%). The crude compound was used in subsequent steps. ¹H-NMR (CDCl₃): δ 7.49-7.45 (m, 2H), 7.10-7.06 (m, 2H), 6.27 (br s, 1H), 3.61 (q, J=6.8 Hz, 2H), 2.86 (t, J=6.8 Hz, 2H). MS (ESI) (M+H)⁺=296/298.

### Compound 8b: 7-Bromo-1,2,3,4-tetrahydro-2-(trifluoroacetyl)isoquinoline

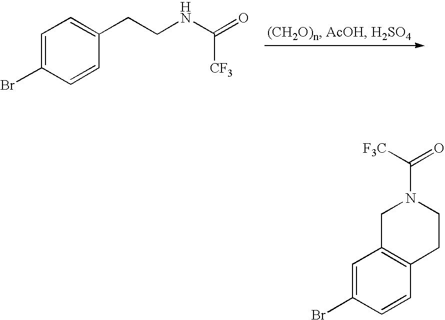

A mixture of glacial acetic acid (5.1 mL) and concentrated sulfuric acid (3.4 mL) was added to a mixture of N-[2-(4-bromophenyl)ethyl]-2,2,2-trifluoroacetamide (0.903 g, 3.05 mmol) and paraformaldehyde (0.147 g, 4.88 mmol equiv. of formaldehyde). The reaction was stirred at room temperature for 20 h, and then poured into 65 mL of cold water. The aqueous solution was extracted with EtOAc (3×25 mL). The combined organic phases were washed with saturated NaHCO₃ (16 mL) and water (2×35 mL), and then dried over Na₂SO₄, filtered, and concentrated in vacuo. The residue was purified by column chromatography (4:1 Hexanes:EtOAc) to provide the title compound (0.885 g, 94%) as a colorless oil. Due to hindered rotation about the amide bond, rotamers were observed in the ¹H-NMR spectrum. ¹H-NMR (CDCl₃): ε 7.38-7.27 (m, 2H), 7.06 (d, J=9.6 Hz, 0.36H), 7.04 (d, J=8.4 Hz, 0.64H), 4.76 (s, 1.3H), 4.71 (s, 0.7H), 3.88 (t, J=6.4 Hz, 0.7H), 3.84 (t, J=6.4 Hz, 1.3H), 2.91 (t, J=5.6 Hz, 1.3H), 2.90 (t, J=6.4 Hz, 0.7H). MS (ESI) (M+H)⁺=308/310.

### Compound 8c: 1,2,3,4-Tetrahydro-7-12-(trifluoromethyl)phenyl]isoquinoline

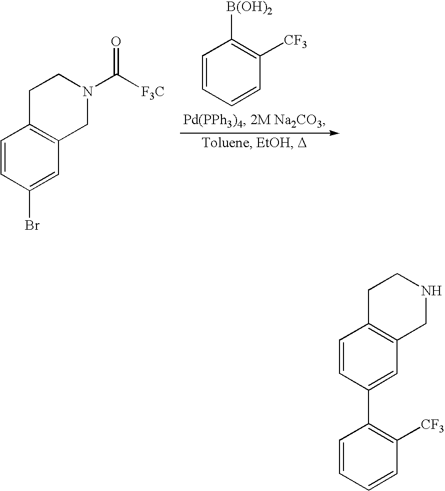

Following General Procedure 1, 7-bromo-1,2,3,4-tetrahydro-2-(trifluoroacetyl)isoquinoline (0.468 g, 1.52 mmol) was reacted with [2-(trifluoromethyl)phenyl]boronic acid (0.433 g, 2.28 mmol) to provide the title compound (0.387 g, 92%) following purification by column chromatography (85:15 CH₂Cl₂:MeOH+0.1% conc. NH₃). ¹H-NMR (CDCl₃): δ 7.74 (d, J=8.0 Hz, 1H), 7.54 (t, J=7.6 Hz, 1H), 7.45 (t, J=8.0 Hz, 1H), 7.31 (d, J=7.6 Hz, 1H), 7.12 (collapsed AB_{q}, 2H), 6.99 (s, 1H), 4.05 (s, 2H), 3.20 (t, J=5.6 Hz, 2H), 2.86 (t, J=6.0 Hz, 2H, 2.43 (br s, 1H). MS (ESI) (M+H)⁺=278.

### Compound 8d: 3,4-Dihydro-α-phenyl-7-[2-(trifluoromethyl)phenyl]-2(1H)-isoquinolineethanol

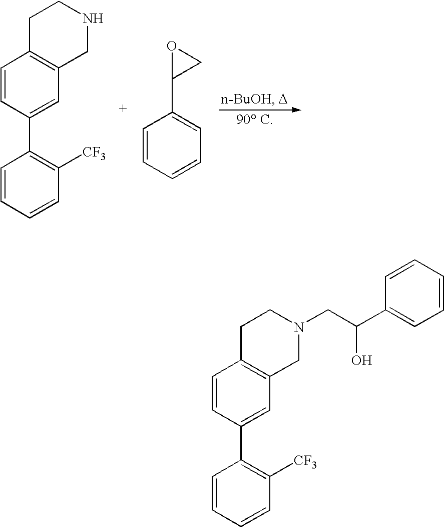

Following General Procedure 5, 1,2,3,4-tetrahydro-7-[2-(trifluoromethyl)phenyl]-isoquinoline (0.0509 g, 0.184 mmol) and 2-(phenyl)oxirane (0.021 mL, 0.0877 mmol) were combined and heated at 90° C. for 14 h. The crude product was purified by reverse phase HPLC (gradient 20-60% CH₃CN in H₂O) to provide the title compound (0.0138 g, 15%) as its TFA salt. This material was lyophilized from H₂O/acetonitrile to produce a white, hygroscopic solid. Due to quaternization of the stereogenic nitrogen atom, a mixture of 2 diastereomeric salts was obtained. ¹H-NMR (CD₃OD): δ 7.80 (d, J=8.0 Hz, 1H), 7.67 (t, J=7.6 Hz, 1H), 7.57 (t, J=7.6 Hz, 1H), 7.53-7.47 (m, 2H), 7.44-7.32 (m, 51H), 7.29 (d, J=8.4 Hz, 1H), 7.27-7.14 (br m, 1H), 5.25 (dd, J=3.6 Hz, J=10.4 Hz, 1H), 4.88-4.43 (br m, 2H), 4.13-3.90 (br m, 1H), 3.62-3.14 (br m, 5H). MS (ESI) (M+H)⁺=398. Anal. Calcd for C₂₄H₂₂F₃NO+1.1 TFA: C, 60.19; H, 4.45; N, 2.68. Found: C, 60.16; H, 4.38; N, 2.61.

### Example 9

### 1-(2-Fluoro-4-nitrophenoxy)-3-[methyl[2′-(trifluoromethyl)[1,1′-biphenyl]-4-yl]amino]-2-propanol

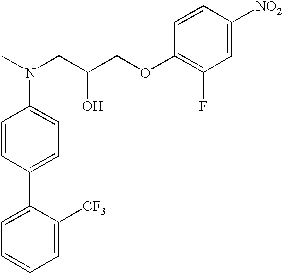

### Compound 9a: 2′-(Trifluoromethyl)-[1,1′-biphenyl]-4-amine

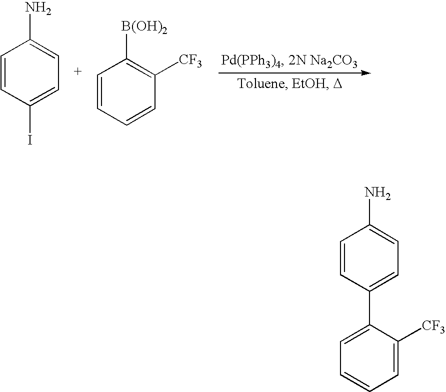

Following General Procedure 1, 4-iodoaniline(1.00 g, 4.57 mmol), 2-(trifluoromethyl)phenylboronic acid (1.302 g, 6.86 mmol), Pd(PPh₃)₄ (0.265 g, 0.23 mmol), and 2 M Na₂CO₃ (16 mL, 32 mmol) were combined. Following the usual work-up, silica gel column chromatography (9:1 Hexanes:EtOAc) provided the title compound (0.476 g, 44%). ¹H-NMR (CDCl₃): δ 7.71 (dd, J=0.4 Hz, J=7.8 Hz, 1H), 7.52 (t, J=7.4 Hz, 1H), 7.41 (t, J=7.8 Hz, 1H), 7.32 (dd, J=0.4 Hz, J=7.6 Hz, 1H), 7.12 (d, J=8.2 Hz, 1H), 6.73-6.69 (m, 2H), 3.73 (br s, 2H). MS (ESI) (M+H)⁺=238.

### Compound 9b: Methyl[2′-(trifluoromethyl)[1,1′-biphenyl]-4-yl]carbamate

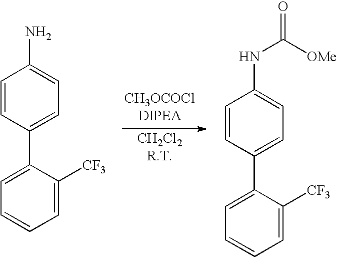

To a solution of 2′-(trifluoromethyl)-[1,1′-biphenyl]-4-amine (0.476 g, 2.01 mmol) and DIPEA (0.45 mL, 2.61 mmol) in CH₂Cl₂ (4.5 mL) maintained at 0° C. was added methylchloroformate (0.17 mL, 2.21 mmol). The reaction was allowed to slowly warm to room temperature, stirred overnight, diluted with CH₂Cl₂ (15 mL), and washed with 1 N HCl (2×20 mL) and brine (1×20 mL). The organic layer was then dried over Na₂SO₄, filtered, and concentrated in vacuo to provide the title compound (0.563 g, 95%) as a beige solid. ¹H-NMR (CDCl₃): δ 7.74.(dd, J=0.6 Hz, J=7.8 Hz, 1H), 7.50 (t, J=7.8 Hz, 1H), 7.47-7.42 (overlapping d and t, J=8.0 Hz for d and J=8.4 Hz for t, 3H), 7.32 (d, J=8.0 Hz, 1H), 7.28 (d, J=8.4 Hz, 2H), 6.69 (br s, 1H), 3.80 (s, 3H). MS (ESI) (M+H)⁺=296.

### Compound 9c: N-Methyl-2′-(trifluoromethyl)-[1,1′-biphenyl]-4-amine

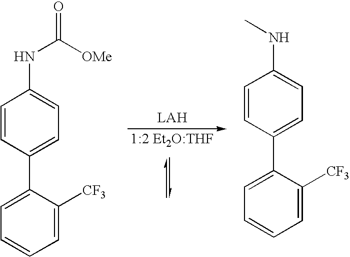

To a solution of methyl [2′-(trifluoromethyl)[1,1]-biphenyl]-4-yl]carbamate (0.554 g, 1.88 mmol) in 1:2 dry Et₂O:THF (30 mL) was added LAH in Et₂O (2.82 mL, 2.82 mmol) dropwise. The reaction was refluxed for 4 hrs, cooled down to room temperature, diluted with Et₂O (40 mL), and quenched with Na₂SO₄.5H₂O (2 g). The reaction mixture was stirred until the solution turned clear, filtered, and concentrated in vacuo to provide the title compound (0.409 g, 87%) as a yellow oil. ¹H-NMR (CDCl₃): δ 7.71 (d, J=8.2 Hz, 1H), 7.52 (t, J=7.6 Hz, 1H), 7.40 (t, J=7.6 Hz, 1H), 7.33 (d, J=7.4 Hz, 1H), 7.17 (d, J=8.2 Hz, 2H), 6.64 (d, J=8.8 Hz, 2H), 2.88 (s, 3H). MS (ESI) (M+H)⁺=252.

### Compound 9d: 1-(2-Fluoro-4-nitrophenoxy)-3-[methyl[2′-(trifluoromethyl)[1,1′-biphenyl]-4-yl]amino]-2-propanol

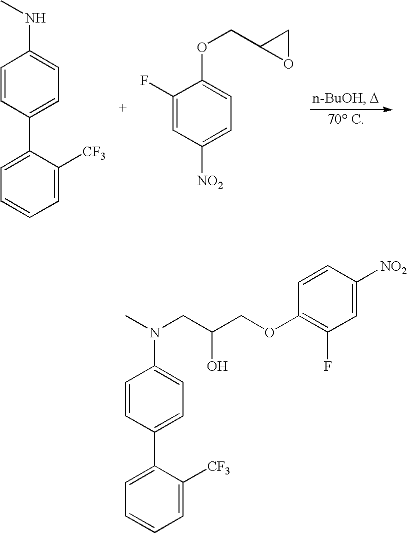

Following General Procedure 5, N-methyl-2′-(trifluoromethyl)-[1,1′-biphenyl]-4-amine (0.100 g, 0.40 mmol) and 2-[(2-fluoro-4-nitrophenoxy)methyl]oxirane (0.085 g, 0.33 mmol) were combined and heated at 70° C. for 24 h. The crude product was purified by reverse phase HPLC (gradient 40-80% CH₃CN in H₂O) to provide the title compound (0.077 g, 42%) as its TFA salt. This material was lyophilized from H₂O/CH₃CN to produce a yellow solid. ¹H-NMR (CD₃OD): δ 8.07-8.02 (m, 2H), 7.71 (d, J=7.6 Hz, 1H), 7.57 (t, J=7.4 Hz, 1H), 7.46 (t, J=7.6 Hz, 1H), 7.29-7.49 (m, 4H), 7.03 (br d, J=7.6 Hz, 2H), 4.24-4.14 (m, 3H), 3.79 (dd, J=5.0 Hz, J=14.2 Hz, 1H), 3.57 (dd, J=7.2 Hz, J=14.4 Hz, 1H), 3.14 (s, 3H). MS (ESI) (M+H)⁺=465. Anal. Calcd for C₂₃H₂F₄N₂O₄+0.2 H₂O+0.3 TFA: C, 56.44; H, 4.15; N, 5.58. Found: C, 56.41; H, 4.05; N, 5.53.

### Example 10

### α-[(2-Fluoro-4-nitrophenoxy)methyl]-1,3-dihydro-5-[2-(trifluoromethyl)phenyl]-2H-isoindole-2-ethanol

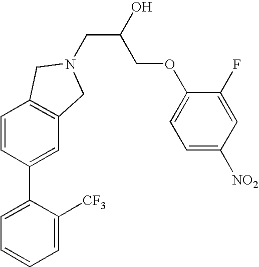

### Compound 10a: 5Bromo-2,3-dihydro-1H-isoindole

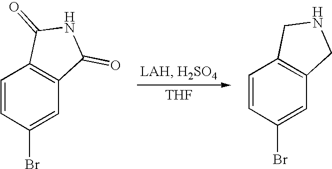

A solution of LiAlH₄ (8.8 mL of 1 M solution in Et₂O, 8.8 mmol) in dry THF (13 mL) was cooled to 0° C. Concentrated H₂SO₄ (0.42 g, 4.3 mmol) was added dropwise, and the resulting mixture was stirred at 0° C. for 30 min. 5-Bromo-1H-isoindole-1,3(2H)-dione (0.409 g, 1.81 mmol) was added in portions over 15 minutes, and the reaction was allowed to warm to room temperature when the addition was complete. The reaction was stirred at room temperature for 2.5 h, and then cooled back to 0° C. and quenched by the addition of MeOH (2 mL). Et₂O was added (50 mL), followed by Na₂SO₄.10H₂O. The mixture was stirred vigorously until the organic layer was clear. The mixture was then filtered and concentrated in vacuo. Purification by column chromatography (4:1 CH₂Cl₂:MeOH+0.1% conc. NH₃) provided the title compound (0.128 g, 36%). ¹H-NMR (CDCl₃): δ 7.38 (s 1H), 7.33 (d, J=7.6 Hz, 1H), 7.12 (d, J=8.0 Hz, 1H), 4.21 (s, 2H), 4.17 (s, 2H), 2.09 (s, 1H). MS (ESI) (M+H)⁺=198/200.

### Compound 10b: 2,3-Dihydro-5-[2-(trifluoromethyl)phenyl]-1H-isoindole

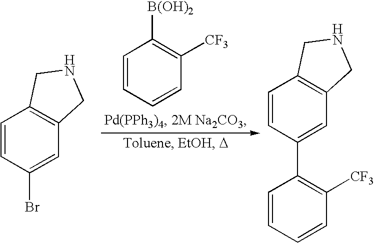

Following General Procedure 1, 5-bromo-2,3-dihydro-1H-isoindole (0.128 g, 0.647 mmol) was reacted with [2-(trifluoromethyl)phenyl]boronic acid (0.184 g, 0.971 mmol) to provide the title compound (0.124 g, 73%) following purification by column chromatography (85:15 CH₂Cl₂:MeOH+0.1% conc. NH₃). ¹H-NMR (CDCl₃): δ 7.74 (d, J=8.0 Hz, 1H), 7.55 (t, J=8.4 Hz, 1H), 7.46 (t, J=7.6 Hz, 1H), 7.32 (d, J=7.2 Hz, 1H), 7.28 (d, J=7.6 Hz, 1H), 7.21 (s, 1H), 7.17 (d, J=8.0 Hz, 1H), 4.30 (s, 2H), 4.29 (s, 2H), 2.34 (br s, 1H). MS (ESI) (M+H)⁺=264.

### Compound 10c: α-[(2-Fluoro-4-nitrophenoxy)methyl]-1,3-dihydro-5-[2-(trifluoromethyl)phenyl]-2H-isoindole-2-ethanol

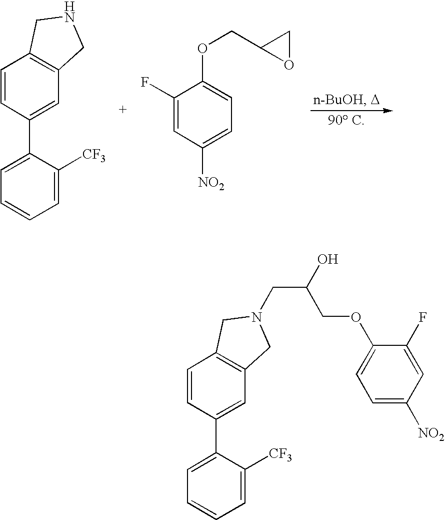

Following General Procedure 5, 2,3dihydro-5-[2-(trifluoromethyl)phenyl]-1H-isoindole (0.0585 g, 0.222 mmol) and 2-[(2-fluoro-4-nitrophenoxy)methyl]-oxirane(0.0474 g, 0.222 mmol) were combined and heated at 90° C. for 14 h. The crude product was purified by reverse phase HPLC (gradient 20-65% CH₃CN in H₂O) to provide the title compound (0.0374 g, 29%) as its TFA salt. This material was lyophilized from H₂O/acetonitrile to produce a white, hygroscopic solid. Due to quaternization of the stereogenic nitrogen atom, a mixture of 2 diastereomeric salts was obtained. ¹H-NMR (CD₃OD): δ 8.13 (ddd, J=1.6 Hz, J=2.8 Hz, J=9.2 Hz, 1H), 8.09 (dd, J=2.8 Hz, J=11.2 Hz, 1H), 7.81 (d, J=7.6 Hz, 1H), 7.68 (t, J=7.2 Hz, 1H), 7.59 (t, J=7.6 Hz, 1H), 7.50 (d, J=8.0 Hz, 1H), 7.42-7.33 (m, 4H), 5.08-4.74 (br s, 4H), 4.52 (sextet, J=4.8 Hz, 1H), 4.30 (d, J=4.8 Hz, 2H), 3.79-3.68 (m, 2H). MS (ESI) (M+H)⁺=477. Anal. Calcd for C₂₄H₂₀F₄N₂O₄+0.6 TFA+2.5 H₂O: C, 51.31; H, 4.37; N, 4.75. Found: C, 51.29; H, 4.38; N, 4.54.

### Example 11

### 1-(2-Fluoro-4-nitrophenoxy)-3-[methyl[[6-[2-(trifluoromethyl)phenyl]-3-pyridinyl]methyl]amino]-2-propanol

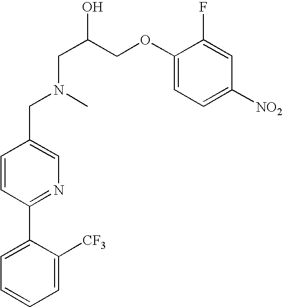

### Compound 11a: N-Methyl-6-[-2-(trifluoromethyl)phenyl]-3-pyridinemethanamine

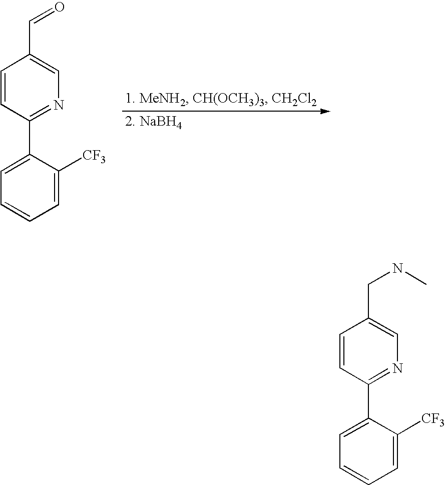

6-[2-(Trifluoromethyl)phenyl]-3-pyridinecarboxaldehyde (0.360 g, 1.43 mmol) was treated according to General Procedure 3 to provide the title compound (0.312 g, 91%). The crude material was of sufficient purity (>90%) to be used in subsequent steps. ¹H-NMR (CDCl₃): δ 8.62 (d, J=1.6 Hz, 1H), 7.76 (d, J=7.6 Hz, 1H), 7.73 (d, J=2.0 Hz, 1H), 7.61 (t, J=7.6 Hz, 1H), 7.54-7.48 (m, 2H), 7.40 (d, J=8.0 Hz, 1H), 3.84 (s, 2H), 2.50 (s, 3H). MS (ESI) (M+H)⁺=267.

### Compound 11b: 1-(2-Fluoro-4-nitrophenoxy)-3-[methyl[[6-[2-(trifluoromethyl)phenyl]-3-pyridinyl]methyl]amino]-2-propanol

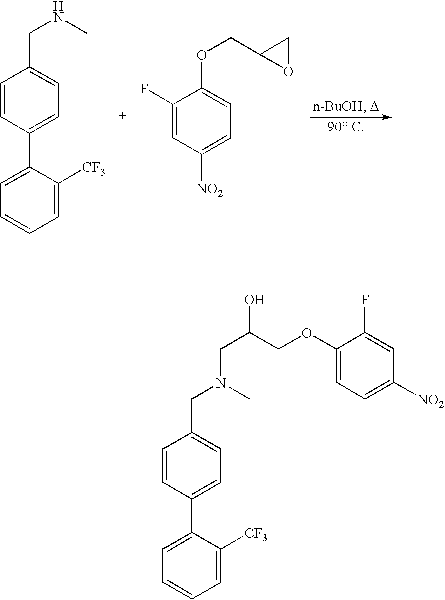

Following General Procedure 5, N-methyl-6-[2-(trifluoromethyl)phenyl]-3-pyridinemethanamine (0.100 g, 0.38 mmol) and 2-[(2-fluoro-4-nitrophenoxy)methyl]oxirane (0.094 g, 0.38 mmol) were combined and heated at 90° C. for 24 h. The crude product was purified by reverse phase HPLC (gradient 20-50% CH₃CN in H₂O) to provide the title compound (0.071 g, 31%) as its TFA salt. This material was lyophilized from H₂O/CH₃CN to produce a white solid. ¹H-NMR (CD₃OD): δ 8.78 (d, J=1.6 Hz, 1H), 8.13-8.03 (m, 3H), 7.84 (d, J=7.6 Hz, 1H), 7.74 (t, J=7.2 Hz, 1H), 7.67 (d, J=8.0 Hz, 1H), 7.64 (d, J=8.0 Hz, 1H), 7.51 (d, J=7.6 Hz, 1H), 7.30 (t, J=8.6 Hz, 1H), 4.66 (br s, 1H), 4.53 (br s, 2H), 4.23 (d, J=4.8 Hz, 2H), 3.43 (t, J=10.0 Hz, 2H), 2.99 (s, 3H). MS (ESI) (M+H)⁺=480. Anal. Calcd for C₂₃H₂₁F₄N₃O₄+0.8 H₂O+1.2 TFA: C, 48.37; H, 3.80; N, 6.66. Found: C, 48.37; H, 3.70; N, 6.79.

### Example 12

### α-[[Methyl-[[6-[2-(trifluoromethyl)phenyl]-3-pyridinyl]methyl]amino]methyl]-benzenemethanol

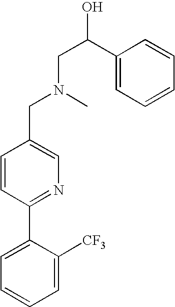

### Compound 12a: Methyl 6-[2-(trifluoromethyl)phenyl]-3-pyridinecarboxylate and Ethyl 6-[2-(trifluoromethyl)phenyl]-3-pyridinecarboxylate

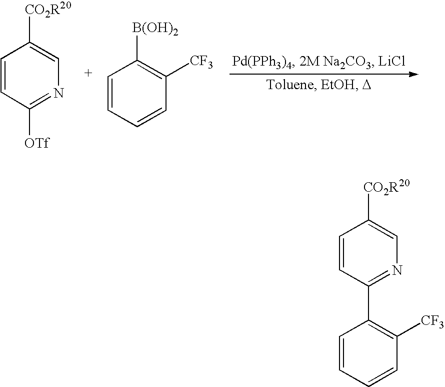 R²⁰=Methyl, or Ethyl

A solution of [2-(trifluoromethyl)phenyl]boronic acid (2.27 g, 12.0 mmol) in ethanol (30 mL) was added to a mixture of methyl 6-[[(trifluoromethyl)sulfonyl]oxy]-3-pyridinecarboxylate (2.27 g, 7.96 mmol), LiCl (1.01 g, 23.9 mmol), Pd(PPh₃)₄ (0.46 g, 0.40 mmol), toluene (120 mL), and 2 M Na₂CO₃ (12 mL). The resulting mixture was heated at reflux for 18 h. The reaction was then concentrated in vacuo, and the residue was diluted with water (60 mL). The aqueous phase was extracted with EtOAc (3×60 mL). The combined organic phases were then washed with brine (80 mL), dried over Na₂SO₄, filtered, and concentrated in vacuo. The residue was purified by column chromatography (4:1 Hexanes:EtOAc) to provide the title compound as a 1:1.4 mixture of the methyl and ethyl esters (1.59 g, 69%). Methyl ester: ¹H-NMR (CDCl₃): δ 9.30 (dd, J=0.8 Hz, J=2.0 Hz, 1H), 8.37 (dd, J=2.4 Hz, J=7.2 Hz, 1H), 7.80 (dd, J=0.8 Hz, J=8.0 Hz, 1H), 7.65 (t, J=7.6 Hz, 1H), 7.67-7.50 (m, 3H), 4.00 (s, 3H). MS (ESI) (M+H)⁺=282. Ethyl ester: ¹H-NMR (CDCl₃): δ 9.29 (dd, J=0.8 Hz, J=2.4 Hz, 1H), 8.37 (dd, J=2.4 Hz, J=8.4 Hz, 1H), 7.79 (dd, J=0.8 Hz, J=8.4 Hz, 1H), 7.65 (t, J=7.6 Hz, 1H), 7.60-7.50 (m, 3H), 4.45 (q, J=7.2 Hz, 2H), 1.44 (t, J=7.2 Hz, 3H). MS (ESI) (M+H)⁺=296.

### Compound 12b: 6-[2-(Trifluoromethyl)phenyl]-3-pyridinecarboxaldehyde

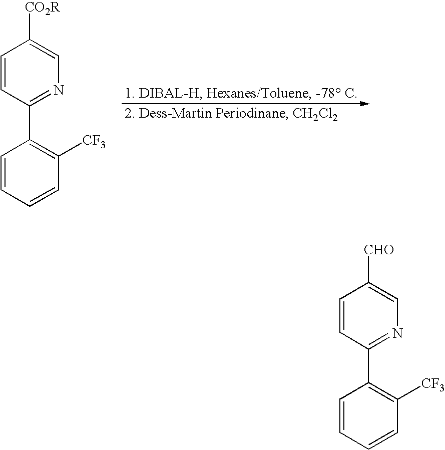

DIBAL-H (12.1 mL of a 1 M solution in hexanes, 12.1 mmol) was added dropwise to a solution of a mixture of methyl and ethyl 6-[2-(trifluoromethyl)phenyl]-3-pyridinecarboxylate (1.59 g of a 1:1.4 mixture, 5.50 mmol) in dry toluene (45 mL) maintained at −78° C. After the addition was complete, the reaction was stirred at —78° C. for 30 min, and then 12 mL of 1 N HCl was added cautiously and the mixture was allowed to warm to room temperature. Additional water (30 mL) was added, the layers were separated, and the aqueous phase was extracted with EtOAc (3×60 mL). The combined organic phases were then dried over Na₂SO₄, filtered, and concentrated in vacuo. The residue was dissolved in CH₂Cl₂ (50 mL) and Dess-Martin periodinane (2.36 g, 5.57 mmol) was added in portions. After the addition was complete, the reaction was stirred at room temperature for 2 h. The reaction was then quenched with 1:1 saturated NaHCO₃:saturated Na₂S₂O₃ (40 mL) and stirred for 15 min. The layers were separated, and the aqueous phase was extracted with CH₂Cl₂ (3×40 mL). The combined organic phases were then dried over Na₂SO₄, filtered, and concentrated in vacuo. The residue was purified by column chromatography (3:2 Hexanes:EtOAc) to provide the title compound (1.23 g, 89% for the two steps) as a slightly yellow oil which solidified upon storage in the freezer. ¹H-NMR (CDCl₃): δ 10.19 (s, 1H), 9.16 (dd, J=0.8 Hz, J=2.0 Hz, 1H), 8.25 (dd, J=2.4 Hz, J=8.0 Hz, 1H), 7.81 (d, J=8.0 Hz, 1H), 7.70-7.56 (m, 3H), 7.52 (d, J=7.6 Hz, 1H). MS (ESI) (M+H)⁺=252.

### Compound 12c: α-[[Methyl-[[6-[2-(trifluoromethyl)phenyl]-3-pyridinyl]methyl]amino]methyl]-benzenemethanol

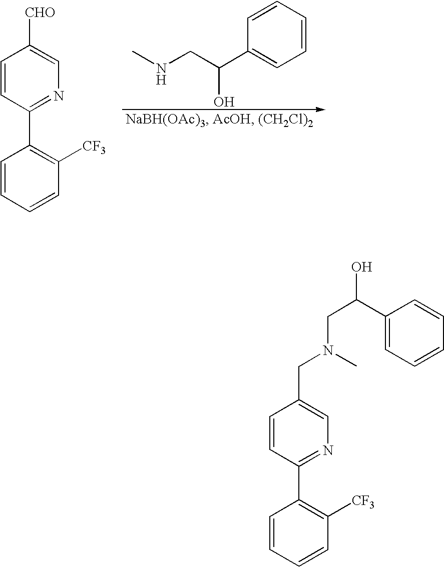

Following General Procedure 4, 6-[2-(trifluoromethyl)phenyl]-3-pyridine-carboxaldehyde (0.166 g, 0.66 mmol), α-[(methylamino)methyl]benzenemethanol (0.100 g, 0.66 mmol), and NaBH(OAc)₃ (0.280 g, 1.32 mmol) were combined. The crude product was purified by reverse phase HPLC (gradient 20-40% CH3CN in H₂O) to provide the title compound (0.279 g, 84%) as its TFA salt. This material was lyophilized from H₂O/acetonitrile to produce a white, hygroscopic solid. ¹H-NMR (CD₃OD): δ 8.81 (s, 1H), 8.14 (d, J=8.0 Hz, 1H), 7.86 (d, J=8.0 Hz, 1H), 7.57 (t, J=7.2 Hz, 1H), 7.72-7.64 (m, 2H), 7.55 (d, J=7.2 Hz, 1H), 7.48-7.31 (m, 5H), 5.17 (br m, 1H), 4.54 (br s, 2H), 3.33 (br s, 2H), 3.03 (br s, 3H). MS (ESI) (M+H)⁺=387. Anal. Calcd for C₂₂H₂₁F₃N₂O+1.2 TFA+1.1 H₂O: C, 53.97; H, 4.53; N, 5.16. Found: C, 54.00; H, 4.43; N, 5.52.

### Example 13

### α-[[Methyl[(2′-nitro[1,1′-biphenyl]-4-yl)methyl]amino]methyl]-benzenemethanol

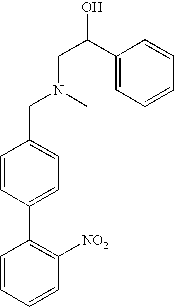

### Compound 13a: α-[[[(4-Bromophenyl)methyl]methylamino]methyl]benzenemethanol

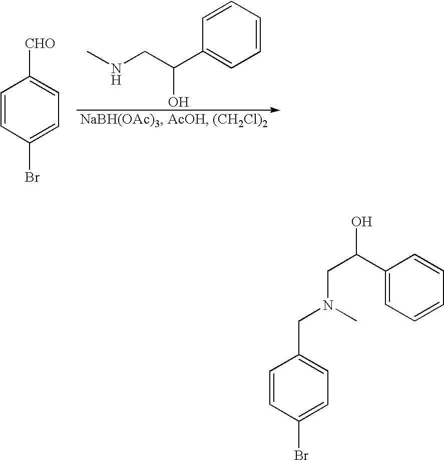

Following General Procedure 4, 4-bromobenzaldehyde (1.22 g, 6.59 mmol), α-[(methylamino)methyl]benzenemethanol (0.500 g, 3.31 mmol), and NaBH(OAc)₃ (1.40 g, 6.61 mmol) were combined. The crude product was purified by flash chromatography (Gradient of 100% CH₂Cl₂ to 9:1 CH₂Cl₂:MeOH+0.1% conc. NH₃) to provide the title compound (0.942 g, 89%). ¹H-NMR (CD₃OD): δ 7.48-7.44 (m, 2H), 7.36-7.32 (m, 4H), 7.32-7.24 (m, 1H), 7.21-7.17 (m, 2H), 4.75 (dd, J=3.6 Hz, J=10.4 Hz, 1H), 3.69 (d, J=13.2 Hz, 1H), 3.48 (d, J=13.2 Hz, 1H), 2.59 (half of d of AB_{q}, J=10.4 Hz, J=12.4 Hz, 1H), 2.52 (half of d of AB_{q}, J=3.2 Hz, J=12.4 Hz, 1H), 2.31 (s, 3H). MS (ESI) (M+H)⁺=320/322.

### Compound 13b: α-[[Methyl[(2′-nitro[1,1′-biphenyl]-4-yl)methyl]amino]methyl]-benzenemethanol

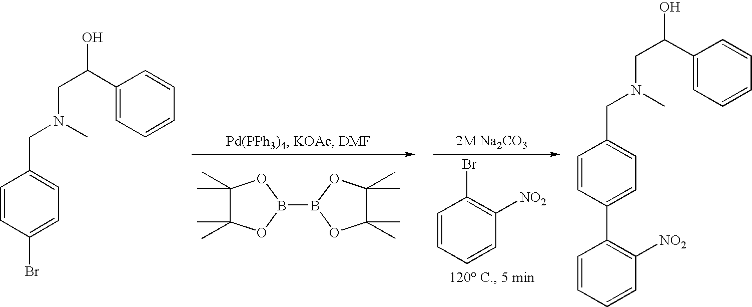

Following General Procedure 2, α-[[[(4-bromophenyl)methyl]methylamino]-methyl]benzenemethanol (0.0530 g, 0.165 mmol) and bis(pinacolato)diboron (0.0462 g, 0.182 mmol) were combined. The resulting boronate ester was used for the reaction with 1-bromo-2-nitrobenzene (0.0669 g, 0.331 mmol) as the second aryl halide. The crude product was purified by reverse phase HPLC (gradient 25-45% CH₃GN in H₂O) to provide the title compound (0.0113 g, 14%) as its TFA salt. This material was lyophilized from H₂O/acetonitrile to produce a white, hygroscopic solid. Due to quaternization of the stereogenic nitrogen atom, a mixture of 2 diastereomeric salts was obtained. ¹H-NMR (CD₃OD): δ 7.96 (d, J=8.0 Hz, 1H), 7.75 (t, J=7.2 Hz, 1H), 7.70-7.57 (b,r m, 3H), 7.57-7.29 (br m, 8H), 5.11 (dd, J=3.6 Hz, J=10.8 Hz, 1H), 4.75 (br d, J=12.8 Hz, 0.51H), 4.54-4.44 (br m, 1H), 4.32 (br d, J=12.0 Hz, 0.5H), 3.48-3.15 (b)r m, 2H), 3.07 (s, 1.5H), 2.91 (s, 1.5H). MS (ESI) (M+H)⁺=363. Anal. Calcd for C₂₂H₂₂N₂O₃+1.1 TFA+1.1 H₂O: C, 57.25; H, 5.02; N, 5.52. Found: C, 57.26; H, 4.97; N. 5.46.

### Example 14

### (α¹S)-aα[[Methyl[[2′-(trifluoromethyl)[1,1′-biphenyl]-4-yl]methyl]amino]methyl]-benzenemethanol

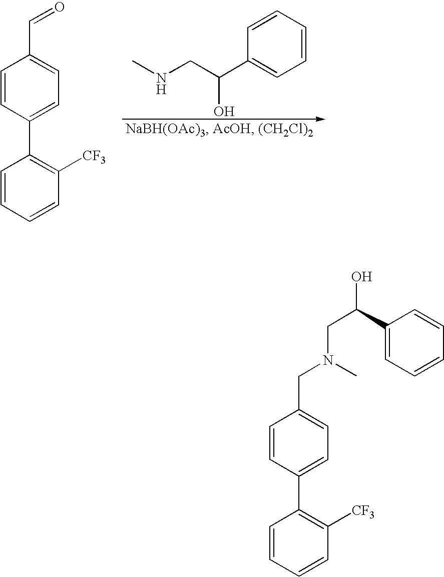

Following General Procedure 4, 2′-(trifluoromethyl)-[1,1′-biphenyl]-4-carboxaldehyde (0.375 g, 1.50 mmol), α-[(methylamino)methyl]benzenemethanol (0.453 g, 3.00 mmol), and NaBH(OAc)₃ (0.636 g, 3.00 mmol) were combined. Following the usual work-up, silica gel column chromatography (9:1 Hexanes:EtOAc) provided the title compound as a racemic mixture. Subsequent chromatography using CHIRALCEL®OD (990:10:1 EtOH:Hex:Et₂NH) gave the title compound. The HCl salt of the title compound (0.0102 g, 3%) was prepared using 1M HCl in Et₂O. This material was lyophilized to produce a white solid. Due to quaternization of the stereogenic nitrogen atom, a mixture of 2 diastereomeric salts was obtained. [α]_{D}²⁴=+44.2° (c=1.02, MeOH). ¹H-NMR (CD₃OD): δ 7.80 (d, J=7.6 Hz, 1H), 7.69-7.56 (overlapping t at 7.67 and m, J=7.4 Hz, 4H), 7.46-7.32 (overlapping d at 7.45 and br m, J=8.0 Hz, 8H), 5.11 (dd, J=6.8 Hz, J=7.2 Hz, 1H), 4.85-4.35 (br m, 2H), 3.26 (br s, 2H), 3.00 (br s, 3H). MS (ESI) (M+H)⁺=386. Anal. Calcd for C₂₃H₂₂F₃NO+0.1 H₂O+1.2 HCl: C, 64.10; H, 5.47; N, 3.25. Found: C, 64.15; H, 5.33; N, 3.80.

### Example 15

### (α¹R)-α-[[Methyl[[2′-(trifluoromethyl)[1,1′-biphenyl]-4-yl]methyl]amino]methyl]-benzenemethanol

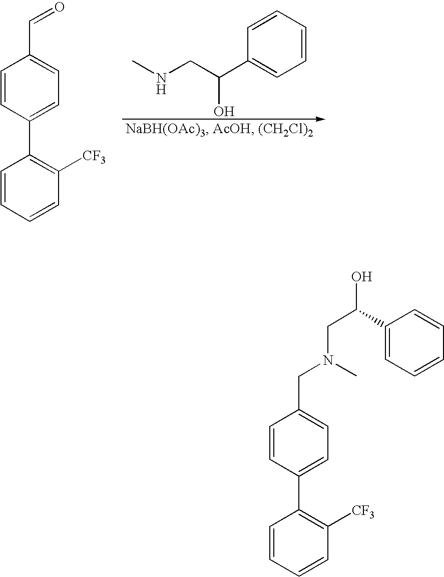

Following General Procedure 4, 2′-(trifluoromethyl)-[1,1′-biphenyl]-4-carboxaldehyde (0.375 g, 1.50 mmol), α-[(methylamino)methyl]benzenemethanol (0453 g, 3.00 mmol), and NaBH(OAc)₃ (0.636 g, 3.00 mmol) were combined. Following the usual work-up, silica gel column chromatography (9:1 Hexanes:EtOAc) provided the title compound as a racemic mixture. Subsequent chromatography using CHIRALCEL®OD (990:10:1 EtOH:Hex:Et₂NH) gave the title compound. The HCl salt of the title compound (0.0056 g, 2%) was prepared using 1M HCl in Et₂O. This material was lyophilized to produce a white solid. Due to quaternization of the stereogenic nitrogen atom, a mixture of 2 diastereomeric salts was obtained. [α]_{D}²⁸=−49.5° (c=0.56, MeOH). ¹H-NMR (CD₃OD): δ 7.79 (d, J=8.0 Hz, 1H), 7.68-7.55 (overlapping t at 7.66 and m, J=7.6 Hz, 4H), 7.45-7.30 (overlapping d at 7.44 and br m, J=7.6 Hz, 8H), 5.10 (dd, J=6.4 Hz, J=7.6 Hz, 1H), 4.84-4.33 (br m, 2H), 3.25 (br s, 2H), 2.98 (br s, 3H). MS (ESI) (M+H)⁺=386. Anal. Calcd for C₂₃H₂₂F₃NO+1.5 HCl: C, 62.77; H, 5.38; N, 3.18. Found: C, 62.89; H, 5.31; N, 3.40.

### Example 16

### α-[[Methyl[[2-methyl-2′-(trifluoromethyl)[1,1′-biphenyl]-4-yl]methyl]amino]methyl]-benzenemethanol

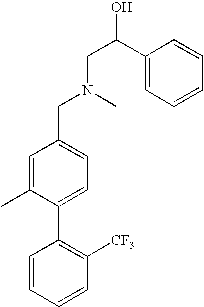

### Compound 16a: 4Formyl-2-methylphenyl trifluoromethanesulfonate

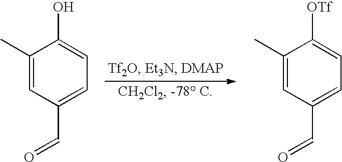

Following General Procedure 7, 4-hydroxy-3-methylbenzaldehyde (0.500 g, 3.67 mmol), DMAP (0.045 g, 0.37 mmol), NEt₃ (1.126 mL, 8.08 mmol), and triflic anhydride (1.139 g, 4.04 mmol) were combined. Silica gel column chromatography (8:2 Hexanes:EtOAc) provided the title compound (0.896 g, 91%) as a white solid. ¹H-NMR (CDCl₃): δ 10.01 (s, 1H), 7.86 (s, 1H), 7.81 (d, J=8.0 Hz, 1H), 7.44 (d, J=7.6 Hz, 1H), 2.48 (s, 3H).

### Compound 16b: 2-Methyl-2′-(trifluoromethyl)-[1,1′-biphenyl]-4-carboxaldehyde

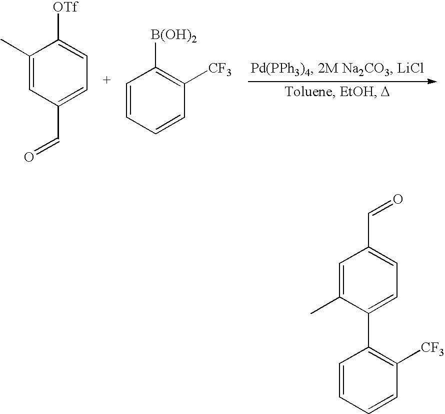

A solution of [2-(trifluoromethyl)phenyl]boronic acid (2.79 g, 14.66 mmol) in ethanol (35 mL) was added to a mixture of 4-formyl-2-methylphenyl trifluoromethanesulfonate (2.62 g, 9.78 mmol), LiCl (1.24 g, 29.33 mmol), Pd(PPh₃)₄ (0.57 g, 0.49 mmol), toluene (145 mL), and 2 M Na₂CO₃ (15 mL). The resulting mixture was heated at reflux for 24 h. The reaction was then concentrated in vacuo, and the residue was diluted with water (60 mL). The aqueous phase was extracted with EtOAc (3×60 mL). The combined organic phases were then washed with brine (80 mL), dried over Na₂SO₄, filtered, and concentrated in vacuo to provide the title compound (2.533 g, 95%). The crude material was of sufficient purity (>85%) to be used in subsequent steps. ¹H-NMR (CDCl₃): δ 10.04 (s, 1H), 7.80-7.78 (overlapping s at 7.78 and d at 7.79, J=7.6 Hz, 2H), 7.73 (d, J=7.6 Hz, 1H), 7.61 (t, J=7.6 Hz, 1H), 7.53 (t, J=7.6 Hz, 1H), 7.32 (d, J=8.0 Hz, 1H), 7.22 (d, J=7.6 Hz, 1H), 2.12 (s, 3H). MS (ESI) (M+H)⁺=265.

### Compound 16c: α-[[Methyl[[2-methyl-2′-(trifluoromethyl)[1,1′-biphenyl]-4-yl]methyl]amino]methyl]-benzenemethanol

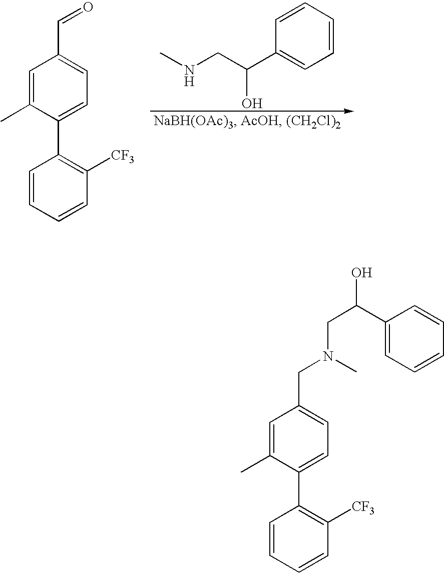

Following General Procedure 4, 2-methyl-2′-(trifluoromethyl)-[1,1′-biphenyl]-4-carboxaldehyde (1.076 g, 3.55 mmol), α-[(methylamino)methyl]benzenemethanol (0.200 g, 1.32 mmol), and NaBH(OAc)₃ (0.562 g, 2.65 mmol) were combined. The crude product was purified by reverse phase HPLC (gradient 30-85% CH₃CN in H₂O) to provide the title compound (0.267 g, 40%) as its TFA salt. This material was lyophilized from H₂O/acetonitrile to produce a white solid. Due to quaternization of the stereogenic nitrogen atom, a mixture of 2 diastereomeric salts was obtained. ¹H-NMR (CD₃OD): δ 7.80 (d, J=7.6 Hz, 1H), 7.66 (t, J=7.6 Hz, 1H), 7.57 (t, J=7.6 Hz, 1H), 7.47-7.22 (overlapping d at 7.26 and br m, J=7.6 Hz, 9H), 5.09 (dd, J=3.2 Hz, J=10.8 Hz, 1H), 4.69 (br d, J=12.4 Hz, 0.5H), 4.47-4.37 (br m, 1H), 4.25 (br d, J=13.2 Hz, 0.5H), 3.41-3.13 (br m, 2H), 3.05 (br s, 1.5H), 2.89 (br s, 1.5H), 2.07-2.05 (overlapping s at 2.07 and s at 2.05, 3H). MS (ESI) (M+H)⁺=400. Anal. Calcd for C₂₄H₂₄F₃NO+0.1 H₂O+1.1 TFA: C, 59.75; H, 4.84; N, 2.66. Found: C, 59.73; H, 4.81; N, 2.75.

### Example 17

### N-(2-Hydroxy-2-phenylethyl)-N-[[2′-(trifluoromethyl)[1,1′-biphenyl]-4-yl]methyl]acetamide

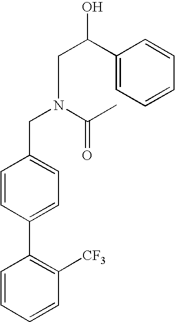

### Compound 17a: α-[[[[2′-(Trifluoromethyl)[1,1′-biphenyl]-4-yl]methyl]amino]methyl]-benzenemethanol

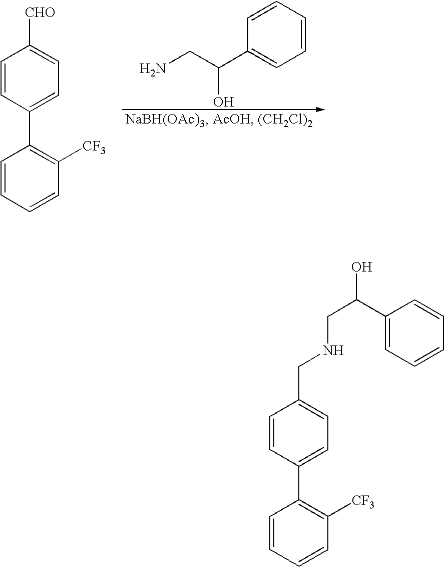

Following General Procedure 4, 2′-(trifluoromethyl)-[1,1′-biphenyl]-4-carboxaldehyde (0.121 g, 0.484 mmol), α-(aminomethyl)benzenemethanol (0.0975 g, 0.711 mmol), and NaBH(OAc)₃ (0.179 g, 0.846 mmol) were combined. The crude product was purified by flash chromatography (9:1 CH₂Cl₂:MeOH) to provide the title compound (0.133 g, 74%). ¹H-NMR (CDCl₃): δ 7.74 (d, J=8.0 Hz, 1H), 7.55 (t, J=7.2 Hz, 1H), 7.46 (t, J=8.0 Hz, 1H), 7.40-7.27 (m, 10H), 4.78 (dd, J=3.6 Hz, J=8.8 Hz, 1H), 3.89 (AB_{q}, J=13.2 Hz, 2H), 2.98 (dd, J=3.6 Hz, J=12.0 Hz, 1H), 2.81 (overlapping dd and br s, J=9.2 Hz, J=12.4 Hz for dd, 3H). MS (ESI) (M+H)⁺=372.

### Compound 17b: N-(2-Hydroxy-2-phenylethyl)-N-[[2′-(trifluoromethyl)[1,1′-biphenyl]-4-yl]methyl]acetamide

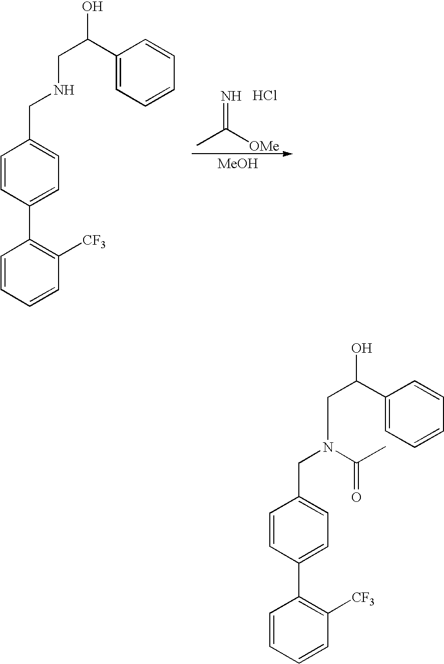

Methyl acetimidate hydrochloride (0.0847 g, 0.773 mmol) was added to a solution of α-[[[[2′-(trifluoromethyl)[1,1′-biphenyl]-4-yl]methyl]amino]methyl]benzenemethanol (0.0287 g, 0.0773 mmol) in dry MeOH (1 mL) maintained at 0° C. The reaction was stirred for 6 d at room temperature, and then an additional portion of methyl acetimidate hydrochloride (0.0500 g, 0.456 mmol) was added. After stirring an additional 7 d, the reaction was concentrated in vacuo. The residue was dissolved in EtOAc (2 mL) and washed with a saturated solution of Na₂CO₃ (1 mL). The aqueous phase was back-extracted with additional EtOAc (3×1 mL). The combined organic phases were dried over Na₂SO₄, filtered, and concentrated in vacuo. The residue was purified by reverse phase HPLC (gradient 20-60% CH₃CN in H₂O) to provide the title compound (0.0105 g, 33%). This material was lyophilized from H₂O/acetonitrile. Due to hindered rotation about the amide bond, rotamers were observed in the ¹H-NMR spectrum. ¹H-NMR (CD₃OD): δ 7.78-7.74 (m, 1H), 7.66-7.60 (m, 1H), 7.56-7.50 (m, 1H), 7.40-7.20 (m, 10H), 5.00 (dd, J=4.8 Hz, J=8.4 Hz, 0.4H), 4.93 (dd, J=4.8 Hz, J=8.0 Hz, 0.6H), 4.88 (d, J=14.8 Hz, 0.6H), 4.72 (d, J=17.2 Hz, 0.4H), 4.61-4.54 (m, 1H), 3.67-3.58 (m, 1H), 3.50 (dd, J=8.4 Hz, J=13.6 Hz, 0.4H), 3.39 (dd J=4.8 Hz, J=15.2 Hz, 0.6H), 2.16 (s, 1.2H), 2.11 (s, 1.8H). MS (ESI) (M+H)⁺=414. Anal. Calcd for C₂₄H₂₂F₃NO₂+0.3 TFA+0.6 H₂O: C, 64.45; H, 5.17; N, 3.06. Found: C, 64.55; H, 5.10; N, 3.50.

### Example 18

### N-(2-Hydroxy-2-phenylethyl)-N-methyl-21′-(trifluoromethyl)-[1,1′-biphenyl]-4-carboxamide

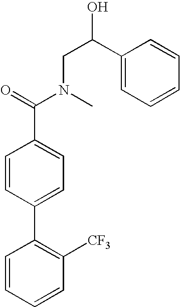

### Compound 18a: 2′-(Trifluoromethyl)-[1,1′-biphenyl]-4-carboxylic acid

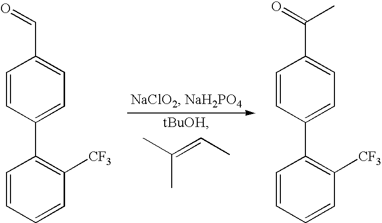

To a solution of 2′-(trifluoromethyl)-[1,1′-biphenyl]-4-carboxaldehyde (0.147 g, 0.59 mol) in t-BuOH (9 mL) and 2-methyl-2-butene (9 mL) was added a solution of NaClO₂ (0.496, 5.50 mmol) and NaH₂PO₄ (0.588 g, 4.9 mmol) in water (6 mL) in four portions over 0.5 h. The resulting reaction mixture was stirred for 5 h at room temperature, concentrated in vacuo, and the residue was diluted with water. The aqueous phase was extracted with CH₂Cl₂ (3×). The product in the combined organic phases was then extracted into 1 N NAOH (3×). The CH₂Cl₂ layer was discarded, the combined aqueous layers were acidified with 1 N HCl, and the product was back extracted with CH₂Cl₂ (3×). The combined organic phases were then dried over Na₂SO₄, filtered, and concentrated in vacuo to provide the title compound (0.125 g, 80%) as a white solid. The crude material was of sufficient purity (>90%) to be used in subsequent steps. ¹H-NMR (CD₃OD): δ 8.06 (d, J=8.0 Hz, 2H), 7.79 (d, J=7.6 Hz, 1H), 7.66 (t, J=7.6 Hz, 1H), 7.57 (t, J=7.6 Hz, 1H), 7.42-7.36 (overlapping d at 7.41 and d at 7.37, J=8.0 Hz for both d, 3H).

### Compound 18b: N-(2-Hydroxy-2-phenylethyl)-N-methyl-2′-(trifluoromethyl)-[1,1′-biphenyl]-4-carboxamide

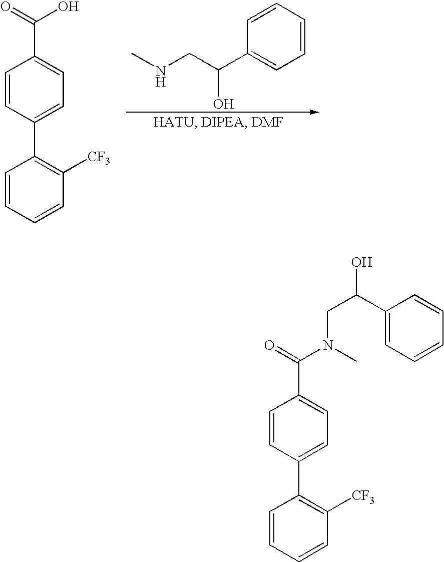

A solution of α-[(methylamino)methyl]benzenemethanol (0.013 g, 0.085 mmol) in DMF (0.5 mL) was added to a solution of 2′-(trifluoromethyl)-[1,1′-biphenyl]-4-carboxylic acid (0.025 g, 0.094 mmol), HATU (0.036 g, 0.094 mmol) and DIPEA (0.022 mL, 0.128 mmol) in DMF (0.5 mL). The reaction was carried out in a 48-well plate. The reaction was stirred overnight at room temperature, concentrated in vacuo, redissolved in EtOAc (1 mL), and washed with 1 N NaOH (3×1 mL) and water (2×1 mL). The organic phase was concentrated in vacuo to provide the title compound (0.027 g, 81%) with >90% purity. Due to hindered rotation about the amide bond, rotamers were observed in the ¹H-NMR spectrum. ¹H-NMR (CD₃OD): δ 7.77 (dd, J=2.0 Hz, J=7.6 Hz, 1H), 7.64 (t, J=7.4 Hz, 1H), 7.54 (t, J=7.6 Hz, 1H), 7.46 (d, J=7.6 Hz, 1H), 7.40-7.22 (d at 7.30, J=8.0 Hz, d at 7.23, J=8.0 Hz, br m, 8H), 7.10-7.08 (m, 1H), 5.08 (t, J=6.6 Hz, 0.5H), 4.81 (t, J=6.4 Hz, 0.5H), 3.74-3.72 (m, 1H), 3.50 (t, J=6.6 Hz, 1H), 3.21 (s, 1.5H), 2.94 (s, 1.5H). MS (ESI) (M+H)⁺=400.

### Example 19

### β-Methoxy-N-methyl-N-[[2′-(trifluoromethyl)[1,1′-biphenyl]-4-yl]methyl]-benzeneethanamine

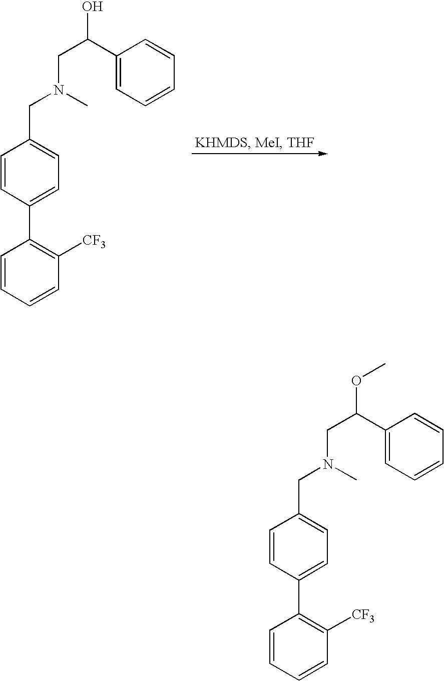

KHMDS (0.45 mL of 0.5M in toluene, 0.225 mmol) was added to a solution of α-[[methyl-[[2′-(trifluoromethyl)-[1,1′-biphenyl]-4-yl]methyl]amino]methyl]benzene-methanol (0.0286 g, 0.0742 mmol) in dry THF (3 mL). The mixture was stirred at room temperature for 20 min, and then neat iodomethane (4.6 μL, 0.074 mmol) was added. The reaction was stirred at room temperature for 19 h, and then quenched by the addition of H2O (3 mL). The layers were separated, and the aqueous phase was extracted with CH₂Cl₂ (4×3 mL). The combined organic phases were then dried over MgSO₄, filtered, and concentrated in vacuo. The residue was purified by reverse phase HPLC (gradient 20-70% CH₃CN in H2O) to provide the title compound (0.0066 g, 17%) as its TFA salt. This material was lyophilized from H₂O/acetonitrile. Due to quaternization of the stereogenic nitrogen atom, a mixture of 2 diastereomeric salts was obtained. ¹H-NMR (CD₃OD): δ 7.82 (d, J=7.6 Hz, 1H), 7.72-7.56 (m, 41H), 7.54-7.30 (m, 8H), 4.78-4.65 (m, 1), 4.62-4.42 (m, 1.5H), 4.36 (br d, J=12.4 Hz, 0.5H), 3.50-3.30 (m, 1.5H), 3.29 (s, 3H), 3.17 (br d, J=12.8 Hz, 0.5H), 3.06 (s, 1.5H), 2.94 (s, 1.5H). MS (ESI) (M+H)⁺=400.

### Example 20

### 3,4-Dihydro-α-phenyl-6-[2-(trifluoromethyl)phenyl]-2(1H)-isoquinolineethanol

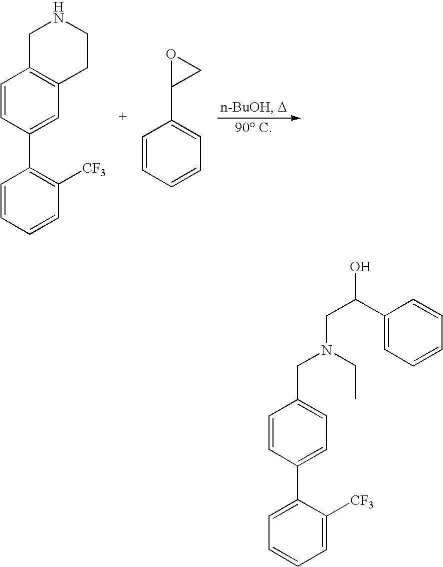

Following General Procedure 5, 1,2,3,4-tetrahydro-6-[2-(trifluoromethyl)phenyl]-isoquinoline (0.0247 g, 0.0891 mmol) and 2-(phenyl)oxirane (0.010 mL, 0.0877 mmol) were combined and heated at 90° C. for 16 h. The crude product was purified by reverse phase HPLC (gradient 25-45% CH₃CN in H₂O) to provide the title compound (0.0111 g, 24%) as its TFA salt. This material was lyophilized from H₂O/acetonitrile to produce a white, hygroscopic solid. Due to quaternization of the stereogenic nitrogen atom, a mixture of 2 diastereomeric salts was obtained. ¹H-NMR (CD₃OD): δ 7.79 (d, J=7.6 Hz, 1H), 7.67 (t, J=7.6 Hz, 1H), 7.57 (t, J=7.6 Hz, 1H), 7.55-7.48 (m, 2H), 7.45-7.39 (m, 2H), 7.38-7.24 (m, 5H), 5.27 (dd, J=3.2 Hz, J=10.0 Hz, 1H), 4.86-4.46 (br m, 2H), 4.12-3.90 (br m, 1H), 3.62-3.12 (br m, 5H). MS (ESI) (M+H)⁺=398. Anal. Calcd for C₂₄H₂₂F₃NO+1.3 TFA+0.5 H₂O: C, 57.60; H, 4.42; N, 2.53. Found: C, 57.60; H, 4.35; N, 2.49.

### Example 21

### α-[[Methyl[[5-[1-methyl-5-(trifluoromethyl)-1H-pyrazol-3-yl]-2-thienyl]methyl]amino]methyl]-benzenemethanol

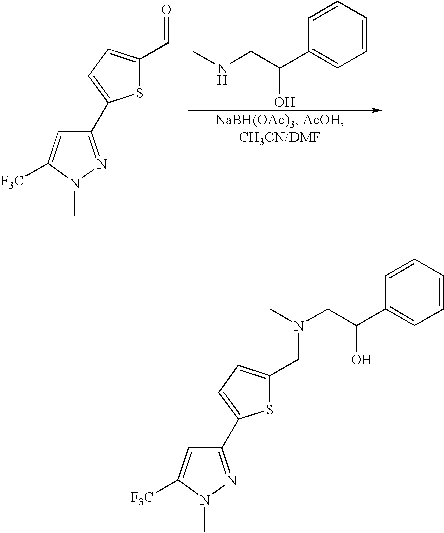

A solution of 5-[1-methyl-5-(trifluoromethyl)-1H-pyrazol-3-yl]-2-thiophenecarboxaldehyde (0.260 g, 0.77 mmol), α-[(methylamino)methyl]benzenemethanol (0.151 g, 0.77 mmol), and acetic acid (0.080 mL) in CH₃CN (4 mL) was stirred for 3 days. A solution of NaBH(OAc)₃ (0.211 g, 3.87 mmol) in DMF (4 mL) was added and the reaction was stirred for 2 days, concentrated in vacuo, redissolved in CH₂Cl₂, and washed with 1 N NaOH. The layers were then filtered through a Hydromatrix® column and the product was eluted with CH₂Cl₂. The organic phase was concentrated in vacuo and purified by reverse phase HPLC (gradient 15-85% CH₃CN in H₂O) to provide the title compound (0.040 g, 10%) as its TFA salt. Due to quatemnization of the stereogenic nitrogen atom, a mixture of 2 diastereomeric salts was obtained. ¹H-NMR (CD₃OD): δ 7.41-7.28 (br m, 7H), 6.81 (s, 1H), 5.10 (dd, J=6.0 Hz, J=7.6 Hz, 1H), 4.80-4.65 (br s at 4.75, s at 4.69, and s at 4.65, 2H), 4.01 (s, 3H), 3.33-3.27 (overlapping s at 3.33 and s at 3.30, 2H), 3.01 (br s, 3H). MS (ESI) (M+H)⁺=396. Anal. Calcd for C₁₉H₂₀F₃N₃OS+0.2 H₂O+1.0 TFA: C, 54.02; H, 4.25; N, 4.85. Found: C, 54.05; H, 4.09; N, 4.85.

### Example 22

### 1-(2-Fluoro-4-nitrophenoxy)-3-[methyl[[2′-(trifluoromethyl)[1,1′-biphenyl]-4-yl]methyl]amino]-2-propanol

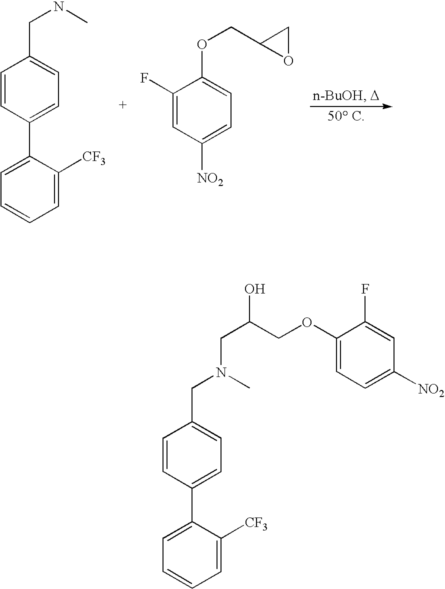

Following General Procedure 5, N-methyl-2′-(trifluoromethyl)-[1,1′-biphenyl]-4-methanamine (0.0800 g of 90% purity, 0.288 mmol) and 2-[(2-fluoro-4-nitrophenoxy)methyl]oxirane (0.0613 g, 0.288 mmol) were combined and heated at 50° C. for 24 h. The crude product was purified by reverse phase HPLC (gradient 20-60% CH₃CN in H₂O) to provide the title compound (0.030 g, 18%) as its TFA salt. This material was lyophilized from H₂O/acetonitrile. Due to quaternization of the stereogenic nitrogen atom, a mixture of 2 diastereomeric salts was obtained. ¹H-NMR (CD₃OD): δ 8.08 (d, J=9.2 Hz, 1H), 8.04 (dd, J=2.0 Hz, J=11.2 Hz, 1H), 7.79 (d, J=8.0 Hz, 1H), 7.66 (t, J=7.6 Hz, 1H), 7.60 (d, J=8.0 Hz, 2H), 7.57 (t, J=7.6 Hz, 1H), 7.44 (d, J=8.0 Hz, 2H), 7.36 (d, J=7.6 Hz, 1H), 7.30 (t, J=8.4 Hz, 1H), 4.72-4.16 (br m at 4.51, br s at 4.21, and underlying br m, 5H), 3.62-3.24 (br s at 3.55, br t at 3.40, br's at 3.28, J=11.2 Hz for t, 2H), 2.97 (br s, 3H). MS (ESI) (M+H)⁺=479. Anal. Calcd for C₂4H₂₂F₄N₂O₄+0.1 H₂O+1.2 TFA: C, 51.39; H, 3.82; N, 4.54. Found: C, 51.34; H, 3.73; N, 4.90.

### Example 23

### 1-[Methyl[[2′-(trifluoromethyl)[1,1′-biphenyl]-4-yl]methyl]amino]-3-(4-nitrophenoxy)2-propanol

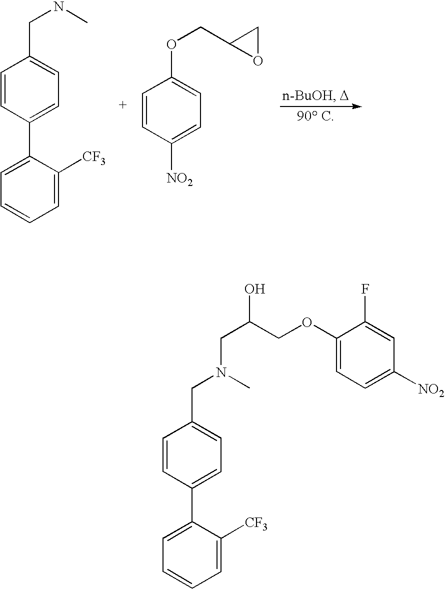

Following General Procedure 5, N-methyl-2′-(trifluoromethyl)-[1,1′-biphenyl]-4-methanamine (0.072 g, 0.29 mmol) and 2-[(4-nitrophenoxy)methyl]-oxirane (0.057 g, 0.29 mmol) were combined and heated at 50° C. for 24 h. The crude product was purified by reverse phase HPLC (gradient 20-60% CH₃CN in H₂O) to provide the title compound (0.034 g, 20%) as its TFA salt. This material was lyophilized from H₂O/CH₃CN to produce a white solid. Due to quaternization of the stereogenic nitrogen atom, a mixture of 2 diastereomeric salts was obtained. ¹H-NMR (CD₃OD): δ 8.20 (d, J=9.2 Hz, 2H), 7.79 (d, J=7.6 Hz, 1H), 7.66 (t, J=7.6 Hz, 1H), 7.62-7.55 (overlapping d at 7.61 and t at 7.57, J=8.4 Hz for d and J=7.6 Hz for t, 3H), 7.44 (d, J=8.0 Hz, 2H), 7.35 (d, J=7.6 Hz, 1H), 7.09 (br d, J=8.4 Hz, 2H), 4.64-4.31 (overlapping br s at 4.64, br s at 4.31, and br m, 3H), 4.13 (br s, 2H), 3.53-3.29 (br s at 3.53, br t at 3.38, and br s at 3.29, J=11.6 Hz for t, 2H), 2.97 (br s, 3H). MS (ESI) (M+H)⁺=461. Anal. Calcd for C₂₄H₂₃F₃N₂O₄+0.2 H₂O+1.0 TFA: C, 54.02; H, 4.25; N, 4.85. Found: C, 54.05; H, 4.09; N, 4.85.

### Example 24

### 1-[[(2′,3′-Dimethyl[1,1′-biphenyl]-4-yl)methyl]methylamino]-3-(2-fluoro-4-nitrophenoxy)-2-propanol

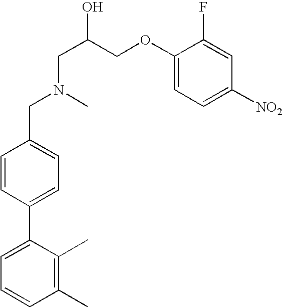

### Compound 24a: 2′,3′-Dimethyl-[1,1′-biphenyl]-4-carboxaldehyde

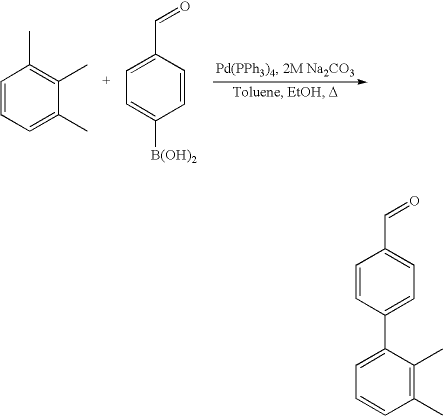

Following General Procedure 1, 1-iodo-2,3-dimethyl-benzene (2.06 g, 8.89 mmol), 4-formylphenylboronic acid (2.00 g, 13.34 mmol), Pd(PPh₃)₄ (0.51 g, 0.44 mmol), and 2 M Na₂CO₃ (31 mL, 62 mmol) were combined. Following the usual work-up provided the title compound (1.05 g, 56%). The crude material was of sufficient purity (>75%) to be used in the subsequent steps. ¹H-NMR (CDCl₃): δ 10.07 (s, 1H), 7.93 (d, J=7.6 Hz, 2H), 7.47 (d, J=8.0 Hz, 2H), 7.22-7.15 (m, 2H), 7.07 (d, J=6.4 Hz, 1H), 2.36 (s, 3H), 2.15 (s, 3H).

### Compound 24b: N,2′,3′-Trimethyl-[1,1′-biphenyl]-4-methanamine

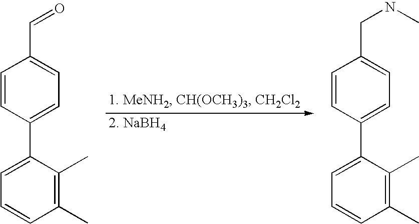

2′,3′-Dimethyl-[1,1′-biphenyl]-4-carboxaldehyde (0.351 g, 1.67 mmol) was treated according to General Procedure 3 to provide the title compound (0.120 g, 40%). The crude material was of sufficient purity (>80%) to be used in subsequent steps. ¹H-NMR (CDCl₃): δ 7.34 (d, J=8.0 Hz, 2H), 7.26 (d, J=8.0 Hz, 2H), 7.15-7.06 (m, 3H), 3.79 (br s, 2H), 2.49 (br s, 3H), 2.33 (s, 3H), 2.15 (s, 3H). MS (ESI) (M+H)⁺=226.

### Compound 24c: 1-[[(2′,3′-Dimethyl[1,1′-biphenyl]-4-yl)methyl]methylamino]-3-(2-fluoro-4-nitrophenoxy)-2-propanol

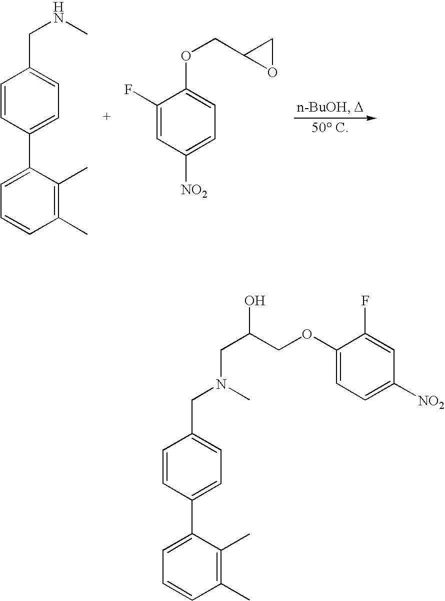

Following General Procedure 5, N,2′,3′-trimethyl-[1,1′-biphenyl]-4-methanamine (0.063 g, 0.30 mmol) and 2-[(2-fluoro-4-nitrophenoxy)methyl]oxirane (0.64 g, 0.38 mmol) were combined and heated at 50° C. for 24 h. The crude product was purified by reverse phase HPLC (gradient 20-60% CH₃CN in H₂O) to provide the title compound (0.027 g, 16%) as its TFA salt. This material was lyophilized from H₂O/CH₃CN to produce a white solid. Due to quaternization of the stereogenic nitrogen atom, a mixture of 2 diastereomeric salts was obtained. ¹H-NMR (CD₃OD): δ 8.09-8.02 (br m, 2H), 7.58 (d, J=8.0 Hz, 2H), 7.40 (d, J=8.0 Hz, 2H), 7.29 (br s, 1H), 7.15 (d, J=6.8 Hz, 1H), 7.10 (t, J=7.4 Hz, 1H), 6.98 (d, J=6.8 Hz, 1H), 4.62, (br s, 0.5H), 4.49 (br s, 2H), 4.27-4.26 (overlapping br s at 4.27 and br s at 4.26, 2.5H), 3.54-3.28 (br s at 3.54, br s at 3.39, and br s at 3.29, 2H), 3.00-2.95 (overlapping br s at 3.00 and br s at 2.95, 3H), 2.32 (s, 3H), 2.11 (s, 3H). MS (ESI) (M+H)⁺=439. Anal. Calcd for C₂₅H₂₇FN₂O₄+0.1 H₂O+1.6 TFA: C, 54.39; H, 4.66; N, 4.50. Found: C, 54.30; H, 4.48; N, 4.41.

### Example 25

### 4-Chloro-α-[[methyl[[2′-(trifluoromethyl)[1,1′-biphenyl]-4-yl]methyl]amino]methyl]-benzenemethanol

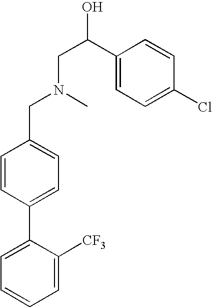

### Compound 25a: 2-(4-Chlorophenyl)oxirane

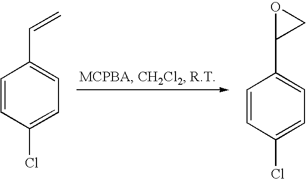

A solution of MCPBA (1.50 g of 60% purity, 5.22 mmol) in CH₂Cl₂ (10 mL) was added to a solution of 1-chloro-4-ethenylbenzene (0.554 g, 4.00 mmol) in CH₂Cl₂ (10 mL) maintained at 0° C. The reaction was allowed to slowly warm to room temperature and stirred for 24 h. The mixture was filtered, and the filtrate was washed with saturated NaHCO₃. The organic phase was dried over Na₂SO₄, filtered, and concentrated in vacuo. The residue was purified by column chromatography (9:1 Hexanes:EtOAc) to provide the title compound (0.198 g, 32%). ¹H-NMR (CDCl₃): δ 7.31 (d, J=8.8 Hz, 2H), 7.20 (d, J=8.8 Hz, 2H), 3.83 (distorted t, J=3.6 Hz, 1H), 3.14 (dd, J=4.0 Hz, J=5.6 Hz, 1H), 2.75 (dd, J=2.4 Hz, J=5.6 Hz, 1H).

### Compound 25b: 4-Chloro-α-[[methyl[[2′-(trifluoromethyl)[1,1′-biphenyl]-4-yl]methyl]amino]methyl]-benzenemethanol

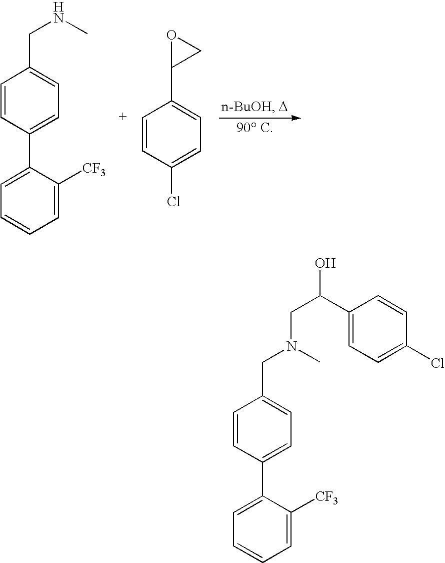

Following General Procedure 5, N-methyl-2′-(trifluoromethyl)-[1,1′-biphenyl]-4-methanamine (0.114 g of 90% purity, 0.387 mmol) and 2-(4-chlorophenyl)oxirane (0.060 g, 0.387 mmol) were combined and heated at 90° C. for 24 h. The crude product was purified by reverse phase HPLC (gradient 25-40% CH₃CN in H₂O) to provide the title compound (0.051 g, 24%) as its TFA salt. This material was lyophilized from H₂O/acetonitrile. Due to quaternization of the stereogenic nitrogen atom, a mixture of 2 diastereomeric salts was obtained. ¹H-NMR (CD₃OD): δ 7.78 (d, J=8.0 Hz, 1H), 7.65 (t, J=7.2 Hz, 1H), 7.62-7.52 (t and overlapping br m, J=7.6 Hz for t, 3H), 7.48-7.31 (m, 7H), 5.12-5.04 (m, 1H), 4.73 (br d, J=13.2 Hz, 0.5H), 4.45 (br m, 1H), 4.27 (br d, J=11.6 Hz, 0.5H), 3.46-3.12 (m, 2H), 3.03 (br s, 1.5H), 2.89 (br s, 1.5H). MS (ESI) (M+H)⁺=420. Anal. Calcd for C₂₃H₂₁ClF₃NO+1.2 TFA+0.1 H₂O: C, 54.62; H, 4.04; N, 2.51. Found: C, 54.63; H. 3.83; N. 2.52.

### Example 26

### 4-Chloro-α-[[[(2′-chloro[1,1′-biphenyl]-4-yl)methyl]methylamino]methyl]-benzenemethanol

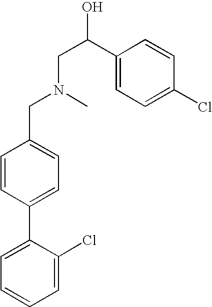

### Compound 26a: 2′-Chloro-N-methyl-[1,1′-biphenyl]-4-methanamine

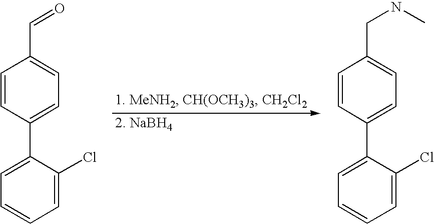

2′-Chloro-[1,1′-biphenyl]-4-carboxaldehyde (0.434 g, 2.00 mmol) was treated according to General Procedure 3 to provide the title compound (0.278 g, 75%). The crude material was of sufficient purity (>75%) to be used in subsequent steps. MS (ESI) (M+H)⁺=232.

### Compound 26b: 4-Chloro-α-[[[(2′-chloro[1,1′-biphenyl]-4-yl)methyl]methylamino]methyl]-benzenemethanol

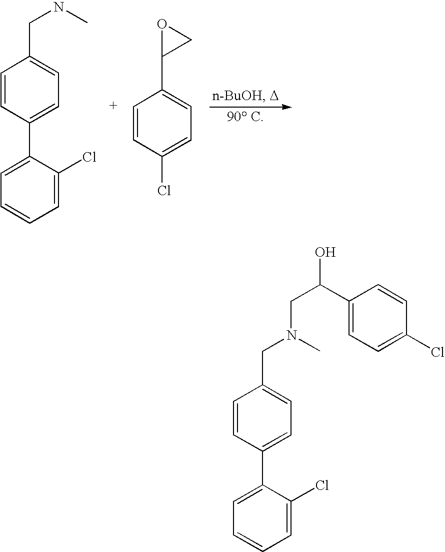

Following General Procedure 5, 2′-chloro-N-methyl-[1,1′-biphenyl]-4-methanamine (0.116 g, 0.50 mmol) and 2-(4-chlorophenyl)oxirane (0.078 g, 0.50 mmol) were combined and heated at 90° C. for 24 h. The crude product was purified by reverse phase HPLC (gradient 25-40% CH₃CN in H₂O) to provide the title compound (0.074 g, 30%) as its TFA salt. This material was lyophilized from H₂O/acetonitrnle. Due to quaternization of the stereogenic nitrogen atom, a mixture of 2 diastereomeric salts was obtained. ¹H-NMR (CD₃OD): δ 7.63-7.50 (br m, 5H), 7.38 (br s, 7H), 5.11 (dd, J=3.4 Hz, J=10.6 Hz, 1H), 4.74 (br d, J=12.0 Hz, 0.5H), 4.47 (br s, 1H), 4.29 (br d, J=12.0 Hz, 0.5H), 3.41-3.17 (br d at 3.42, and br m, J=9.6 Hz for d, 2H), 3.05 (br s, 1.5H), 2.89 (br s, 1.5H). MS (ESI) (M+H)⁺=386. Anal. Calcd for C₂₂H₂₁Cl₂NO+0.1 H₂O+1.1 TFA: C, 56.60; H. 4.38; N, 2.73. Found: C, 56.49; H, 4.28; N, 2.70.

### Example 27

### 1-[[(2′,5′-Dimethyl]1,1′-biphenyl]-4-yl)methyl]methylamino]-3-(2-fluoro-4-nitrophenoxy)-2-propanol

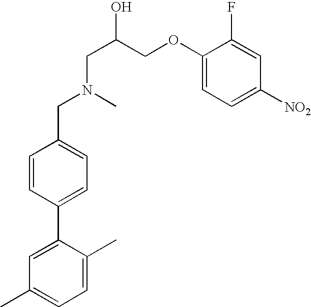

### Compound 27a: 2′,5′-Dimethyl-(1,1′-biphenyl]-4-carboxaldehyde

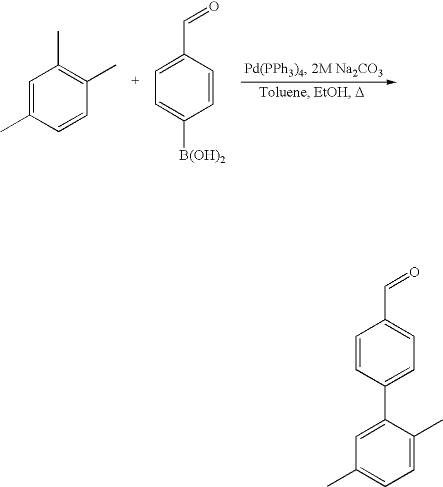

Following General Procedure 1, 2-iodo-1,4-dimethyl-benzene (2.06 g, 8.89 mmol), 4-formylphenylboronic acid (2.00 g, 13.34 mmol), Pd(PPh₃)₄ (0.51 g, 0.44 mmol), and 2 M Na₂CO₃ (31 mL, 62 mmol) were combined. Following the usual work-up provided the title compound (1.67 g, quantitative). The crude material was of sufficient purity (>90%) to be used in the subsequent steps. ¹H-NMR (CDCl₃): δ 10.06 (s, 1H), 7.92 (dd, J=1.8 Hz, J=8.2 Hz, 2H), 7.49 (dd, J=1.6 Hz, J=8.4 Hz, 2H), 7.18 (d, J=7.6 Hz, 1H), 7.12 (d, J=8.4 Hz, 1H), 7.05 (s, 1H1), 2.36 (s, 3H), 2.23 (s, 3H).

### Compound 27b: N,2′,5′-Trimethyl-[1,1′-biphenyl]-4-methanamine

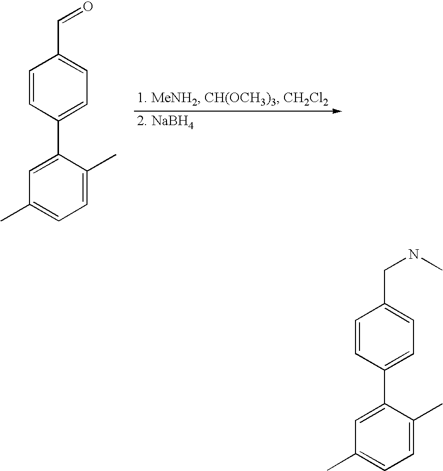

2′,5′-Dimethyl-[1,1′-biphenyl]-4-carboxaldehyde (0.263 g, 1.25 mmol) was treated according to General Procedure 3 to provide the title compound (0.203 g, 80%). The crude material was of sufficient purity (>90%) to be used in subsequent steps. MS (ESI) (M+H)⁺=226.

### Compound 27c: 1-[[(2′,5′-Dimethyl[1,1′-biphenyl]-4-yl)methyl]methylamino]-3-(2-fluoro-4-nitrophenoxy)-2-propanol

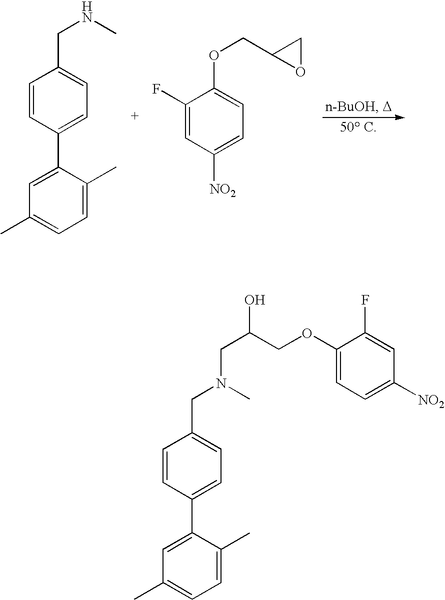

Following General Procedure 5, N,2′,5′-trimethyl-[1,1′-biphenyl]-4-methanamine (0.068 g, 0.30 mmol) and 2-[(2-fluoro-4-nitrophenoxy)methyl]oxirane (0.64 g, 0.38 mmol) were combined and heated at 50° C. for 24 h. The crude product was purified by reverse phase HPLC (gradient 25-40% CH₃CN in H₂O) to provide the title compound (0.056 g, 34%) as its TFA salt. This material was lyophilized from H₂O/CH₃CN to produce a white solid. Due to quaternization of the stereogenic nitrogen atom, a mixture of 2 diastereomeric salts was obtained. ¹H-NMR (CD₃OD): δ 8.08-8.00 (br m, 2H), 7.56 (d, J=8.4 Hz, 2H), 7.40 (d, J=8.4 Hz, 2H), 7.27 (br s, 1H), 7.13 (d, J=7.6 Hz, 1H), 7.05 (d, J=8.0 Hz, 1H), 6.96 (s, 1H), 4.61, (br s, 0.5H), 4.46 (br s, 2H), 4.28-4.18 (overlapping br d at 4.26 and br s at 4.18, J=15.2 Hz, 2.5H), 3.54-3.22 (br d at 3.52, br s at 3.39, and br s at 3.22, J=12.4 Hz, 2H), 2.98-2.91 (overlapping br s at 2.98 and br s at 2.91, 3H), 2.29 (s, 3H), 2.15 (s, 3H). MS (ESI) (M+H)⁺=439. Anal. Calcd for C₂₅H₂₇FN_{2l O}₄+0.4 H₂O+1.2 TFA: C, 56.49; H, 5.02; N, 4.81. Found: C, 56.46; H, 5.01; N, 4.86.

### Example 28

### α-[[[(2′,5′-Dimethyl[1,1′-biphenyl]-4-yl)methyl]methylamino]methyl]-benzenemethanol

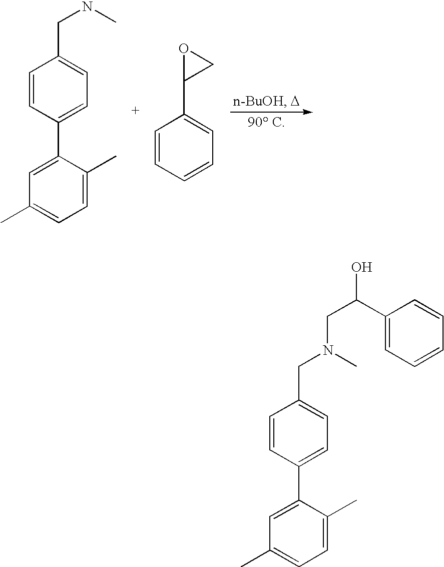

Following General Procedure 5, N,2′,5′-trimethyl-[1,1′-biphenyl]-4-methanamine (0.072 g, 0.32 mmol) and 2-phenyl-oxirane (0.038 g, 0.32 mmol) were combined and heated at 90° C. for 24 h. The crude product was purified by reverse phase HPLC (gradient 25-40% CH₃CN in H₂O) to provide the title compound (0.033 g, 22%) as its TFA salt. This material was lyophilized from H₂O/acetonitrile. Due to quatemization of the stereogenic nitrogen atom, a mixture of 2 diastereomeric salts was obtained. ¹H-NMR (CD₃OD): δ 7.54 (br s, 2H), 7.40-7.31 (br m, 7H), 7.13 (d, J=8.0 Hz, 1H), 7.05 (d, J=7.6 Hz, 1H), 6.98 (s, 1H), 5.08 (dd, J=3.6 Hz, J=10.8 Hz, 1H), 4.71 (br d, J=10.0 Hz, 0.5H), 4.44 (br s, 1H), 4.27 (br d, J=13.2 Hz, 0.5H), 3.41-3.16 (br d at 3.39, and br m, J=12.8 Hz for d, 2H), 3.03 (br s, 1.5H), 2.87 (br s, 1.5H), 2.29 (s, 3H), 2.16 (s, 3H). MS (ESI) (M+H)⁺=346. Anal. Calcd for C₂₄H₂₇NO+0.6 H₂O+1.0 TFA: C, 66.40; H, 6.26; N, 2.98. Found: C, 66.45; H, 6.16; N, 2.68.

### Example 29

### α-[[Methyl[[4-(3-methyl-2-thienyl)phenyl]methyl]amino]methyl]-benzenemethanol

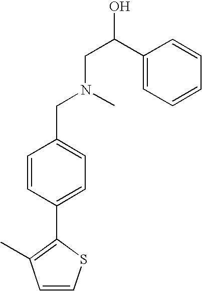

### Compound 29a: 4(3-Methyl-2-thienyl)-benzaldehyde

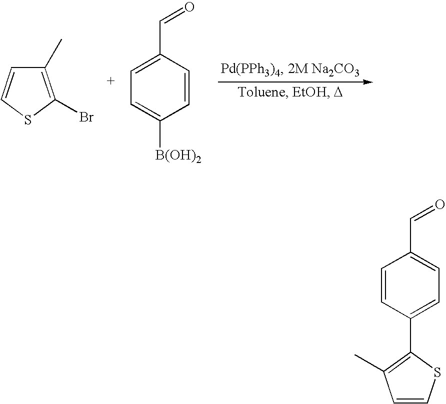

Following General Procedure 1, 2-bromo-3-methyl-thiophene (0.88 g, 4.95 mmol), 4-formylphenylboronic acid (1.11 g, 7.43 mmol), Pd(PPh₃)₄ (0.29 g, 0.25 mmol), and 2 M Na₂CO₃ (15 mL, 35 mmol) were combined. Following the usual work-up provided the title compound (0.579 g, 58%). The crude material was of sufficient purity (>50%) to be used in subsequent steps. ¹H-NMR (CDCl₃): δ 10.04 (s, 1H), 7.92 (d, J=8.4 Hz, 2H), 7.64 (d, J=8.4 Hz, 2H), 7.30 (d, J=5.2 Hz, 1H), 6.97 (d, J=5.2 Hz, 1H), 2.39 (s, 3H).

### Compound 29b: N-Methyl-4-(3-methyl-2-thienyl)-benzenemethanamine

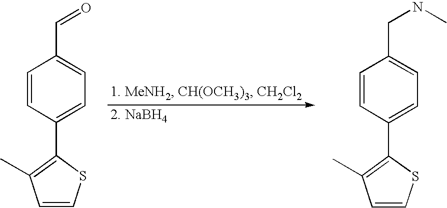

4-(3-Methyl-2-thienyl)-benzaldehyde (0.253 g, 1.25 mmol) was treated according to General Procedure 3 to provide the title compound (0.139 g, 57%). The crude material was of sufficient purity (>90%) to be used in subsequent steps. ¹H-NMR (CDCl₃): δ 7.55 (d, J=8.4 Hz, 1H), 7.42 (d, J=8.4 Hz, 1H), 7.38-7.33 (overlapping d at 7.37, J=8.4 Hz, and d at 7.34, J=8.4 Hz, 2H), 7.18 (d, J=5.2 Hz, 1H), 6.91 (d, J=5.2 Hz, 1H), 3.77 (s, 2H), 2.47 (s, 3H), 2.32 (s, 3H). MS (ESI) (M+H)⁺=218.

### Compound 29c: α-[[Methyl[[4-(3-methyl-2-thienyl)phenyl]methyl]amino]methyl]-benzenemethanol

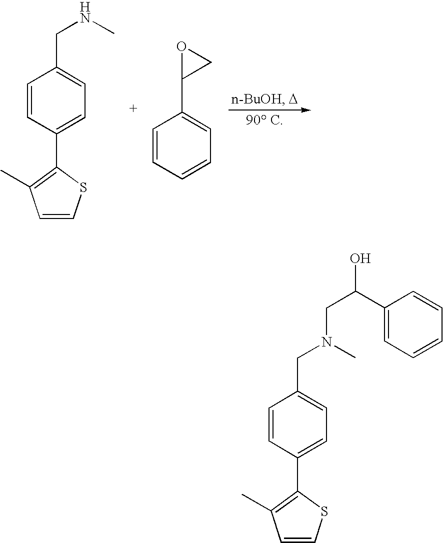

Following General Procedure 5, N-methyl-4-(3-methyl-2-thienyl)-benzenemethanamine (0.109 g, 0,50 mmol) and 2-phenyl-oxirane (0.060 g, 0.50 mmol) were combined and heated at 90° C. for 24 h. The crude product was purified by reverse phase HPLC (gradient 20-30% CH₃CN in H₂O) to provide the title compound (0.032 g, 14%) as its TFA salt. This material was lyophilized from H₂O/acetonitrile. Due to quaternization of the stereogenic nitrogen atom, a mixture of 2 diastereomeric salts was obtained. ¹H-NMR (CD₃OD): δ 7.62-7.57 (m, 4H), 7.42-7.33 (overlapping d at 7.33 and m, J=4.8 Hz for d, 6H), 6.96 (d, J=5.2 Hz, 1H), 5.12 (br s, 1H), 4.73 (br d, J=12.8 Hz, 0.5H), 4.45 (br s, 1H), 4.27 (br d, J=13.2 Hz, 0.5H), 3.43-3.18 (br d at 3.42, J=12.4 Hz, br d at 3.18, J=11.2 Hz, and br m, 2H), 3.04 (s, 1.5H), 2.88 (s, 1.5H), 2.33 (s, 3H). MS (ESI) (M+H)⁺=338. Anal. Calcd for C₂₁H₂₃NOS+0.8 H₂O+1.1 TFA: C, 58.38; H, 5.43; N, 2.93. Found: C, 58.48; H, 5.41; N, 2.93.

### Examples 30-132

Additional exemplary compounds were prepared according to the general procedures and the examples described above. Mass spectra of these compounds were obtained to confirm the formation of these compounds. These exemplary compounds and the mass spectrum results thereof are listed in Table 2 below.

| Example | | MS (ESI) |
|---|---|---|
| No. | Compound Name | (M + H)⁺ |
| 30 | 1-[4-(1,1-Dimethylethyl)phenoxy]-3-[methyl[[2′- | 472 |
| | (trifluoromethyl)[1,1′-biphenyl]-4- | |
| | yl]methyl]amino]-2-propanol | |
| 31 | 1-[4-(1,1-Dimethylethyl)phenoxy]-3-[[(2′- | 434 |
| | methoxy[1,1′-biphenyl]-4-yl)methyl]methylamino]- | |
| | 2-propanol | |
| 32 | β-Ethoxy-N-methyl-N-[[2′-(trifluoromethyl)[1,1′- | 414 |
| | biphenyl]-4-yl]methyl]benzeneethanamine | |
| 33 | N-Methyl-N-[[2′-(trifluoromethyl)[1,1′-biphenyl]-4- | 409 |
| | yl]methyl]glycylglycine, ethyl ester | |
| 34 | N-Ethyl-2-[methyl[[2′-(trifluoromethyl)[1,1′- | 351 |
| | biphenyl]-4-yl]methyl]amino]acetamide | |
| 35 | α-[(2-Fluoro-4-nitrophenoxy)methyl]-3,4-dihydro-7- | 491 |
| | [2-(trifluoromethyl)phenyl]-2(1H)- | |
| | isoquinolineethanol | |
| 36 | α-[[Methyl[(2,2′,5′-trimethyl[1,1′-biphenyl]-4- | 360 |
| | yl)methyl]amino]methyl]benzenemethanol | |
| 37 | 1-[[[2′-Chloro-5′-(trifluoromethyl)[1,1′-biphenyl]-4- | 513 |
| | yl]methyl]methylamino]-3-(2-fluoro-4- | |
| | nitrophenoxy)-2-propanol | |
| 38 | 4′-[[[3-(2-Fluoro-4-nitrophenoxy)-2- | 466 |
| | hydroxypropyl]methylamino]methyl]-6-methoxy- | |
| | [1,1′-biphenyl]-3-carbonitrile | |
| 39 | 1-[[(2′,5′-Dichloro[1,1′-biphenyl]-4- | 479 |
| | yl)methyl]methylamino]-3-(2-fluoro-4- | |
| | nitrophenoxy)-2-propanol | |
| 40 | 1-[[[4-(2-Chloro-3- | 451 |
| | thienyl)phenyl]methyl]methylamino]-3-(2-fluoro-4- | |
| | nitrophenoxy)-2-propanol | |
| 41 | 4′-[[[3-(2-Fluoro-4-nitrophenoxy)-2- | 436 |
| | hydroxypropyl]methylamino]methyl]-[1,1′- | |
| | biphenyl]-2-carbonitrile | |
| 42 | 1-[[(2′-Chloro-5′methyl[1,1′-biphenyl]-4- | 459 |
| | yl)methyl]methylamino]-3-(2-fluoro-4- | |
| | nitrophenoxy)-2-propanol | |
| 43 | 1-[[(5′-Chloro-2′-methyl[1,1′-biphenyl]-4- | 459 |
| | yl)methyl]methylamino]-3-(2-fluoro-4- | |
| | nitrophenoxy)-2-propanol | |
| 44 | 1-(2-Fluoro-4-nitrophenoxy)-3-[methyl[(2′-nitro[1,1′- | 456 |
| | biphenyl]-4-yl)methyl]amino]-2-propanol | |
| 45 | α-[[[[4-(2-Chloro-3- | 358/360 |
| | thienyl)phenyl]methyl]methylamino]methyl]benzenemethanol | |
| 46 | 4′-[[(2-Hydroxy-2- | 343 |
| | phenylethyl)methylamino]methyl]-[1,1′-biphenyl]- | |
| | 2-carbonitrile | |
| 47 | α-[[[(5′-Chloro-2′-methyl[1,1′-biphenyl]-4- | 366/368 |
| | yl)methyl]methylamino]methyl]benzenemethanol | |
| 48 | α-[[Methyl[[2′-methyl-5′-(trifluoromethyl)[1,1′- | 400 |
| | biphenyl]-4- | |
| | yl]methyl]amino]methyl]benzenemethanol | |
| 49 | α-[[[[2′-Chloro-5′-(trifluoromethyl)[1,1′-biphenyl]-4- | 420/422 |
| | yl]methyl]methylamino]methyl]benzenemethanol | |
| 50 | 4′-[[(2-Hydroxy-2- | 373 |
| | phenylethyl)methylamino]methyl]-6-methoxy-[1,1′- | |
| | biphenyl]-3-carbonitrile | |
| 51 | α-[[[(2′-Fluoro[1,1′-biphenyl]-4- | 336 |
| | yl)methyl]methylamino]methyl]benzenemethanol | |
| 52 | α-[[[(2′,5′-Dichloro[1,1′-biphenyl]-4- | 386/388/390 |
| | yl)methyl]methylamino]methyl]-benzenemethanol | |
| 53 | Methyl 3-[4-[[(2-hydroxy-2- | 382 |
| | phenylethyl)methylamino]methyl]phenyl]-2- | |
| | thiophenecarboxylate | |
| 54 | α-[[Methyl[[2′-(1-methylethoxy)[1,1′-biphenyl]-4- | 376 |
| | yl]methyl]amino]methyl]benzenemethanol | |
| 55 | α-[[[(2′-Ethoxy[1,1′-biphenyl]-4- | 362 |
| | yl)methyl]methylamino]methyl]benzenemethanol | |
| 56 | α-[[Methyl[[2′-(2-propenyl)[1,1′-biphenyl]-4- | 358 |
| | yl]methyl]amino]methyl]benzenemethanol | |
| 57 | α-[[[(2′-Cyclopentyl[1,1′-biphenyl]-4- | 386 |
| | yl)methyl]methylamino]methyl]benzenemethanol | |
| 58 | α-[[Methyl-[[5′-methyl-2′-(1-methylethyl)[1,1′- | 374 |
| | biphenyl]-4- | |
| | yl]methyl]amino]methyl]benzenemethanol | |
| 59 | α-[[[(2′-Methoxy-5′-methyl[1,1′-biphenyl]-4- | 362 |
| | yl)methyl]methylamino]methyl]-benzenemethanol | |
| 60 | 1-(2-Fluoro-4-nitrophenoxy)-3-[methyl[[2′-methyl- | 493 |
| | 5′-(trifluoromethyl)[1,1′-biphenyl]-4- | |
| | yl]methyl]amino]-2-propanol | |
| 61 | α-[[[[5-(4-Bromophenyl)-2- | 386/388 |
| | furanyl]methyl]methylamino]methyl]benzenemethanol | |
| 62 | α-[[[[5-(4-Chlorophenyl)-2- | 342 |
| | furanyl]methyl]methylamino]methyl]benzenemethanol | |
| 63 | α-[[Methyl[[5-[3-(trifluoromethyl)phenyl]-2- | 376 |
| | furanyl]methyl]amino]methyl]benzenemethanol | |
| 64 | Methyl 3-[5-[[(2-hydroxy-2- | 372 |
| | phenylethyl)methylamino]methyl]-2-furanyl]-2- | |
| | thiophenecarboxylate | |
| 65 | α-[[Methyl[[4-(3- | 319 |
| | pyridinyl)phenyl]methyl]amino]methyl]benzenemethanol | |
| 66 | 1-[[(2′-Chloro[1,1′-biphenyl]-4- | 438 |
| | yl)methyl]methylamino]-3-[4-(1,1- | |
| | dimethylethyl)phenoxy]-2-propanol | |
| 67 | 1-(4-Chlorophenoxy)-3-[methyl[[2′- | 450 |
| | (trifluoromethyl)[1,1′-biphenyl]-4- | |
| | yl]methyl]amino]-2-propanol | |
| 68 | 1-[Methyl[[2′-(trifluoromethyl)[1,1′-biphenyl]-4- | 416 |
| | yl]methyl]amino]-3-phenoxy-2-propanol | |
| 69 | 1-[[(2′-Methoxy[1,1′-biphenyl]-4- | 423 |
| | yl)methyl]methylamino]-3-(4-nitrophenoxy)-2- | |
| | propanol | |
| 70 | α-[[Methyl[[2′-(trifluoromethyl)[1,1′-biphenyl]-4- | 400 |
| | yl]methyl]amino]methyl]benzeneethanol | |
| 71 | 1-(1,1-Dimethylethoxy)-3-[methyl[[2′- | 396 |
| | (trifluoromethyl)[1,1′-biphenyl]-4- | |
| | yl]methyl]amino]-2-propanol | |
| 72 | Methyl 2-hydroxy-2-methyl-3-[methyl[[2′- | 382 |
| | (trifluoromethyl)[1,1′-biphenyl]-4- | |
| | yl]methyl]amino]propanoate | |
| 73 | (β¹S)-β-[[(2′-Chloro[1,1′-biphenyl]-4- | 372 |
| | yl)methyl]methylamino]-cyclohexanepropanol | |
| 74 | 1-(4-Chlorophenoxy)-3-[[(2′-methyl[1,1′-biphenyl]- | 422 |
| | 4-yl)methyl]-2-propenylamino]-2-propanol | |
| 75 | 1-[[(2′-Methyl[1,1′-biphenyl]-4-yl)methyl]-2- | 388 |
| | propenylamino]-3-phenoxy-2-propanol | |
| 76 | 1-[[(2′-Chloro[1,1′-biphenyl]-4-yl)methyl]-2- | 408 |
| | propenylamino]-3-phenoxy-2-propanol | |
| 77 | 1-Phenoxy-3-[2-propenyl[[2′-trifluoromethyl)[1,1′- | 442 |
| | biphenyl]-4-yl]methyl]amino]-2-propanol | |
| 78 | 1-[[(2′-Chloro[1,1′-biphenyl]-4-yl)methyl]-2- | 476 |
| | propenylamino]-3-(3,4-dichlorophenoxy)-2- | |
| | propanol | |
| 79 | 1-[([1,1′-Biphenyl]-4-ylmethyl)-2-propenylamino]-3- | 419 |
| | (4-nitrophenoxy)-2-propanol | |
| 80 | 1-[[(2′-Methyl[1,1′-biphenyl]-4-yl)methyl]-2- | 433 |
| | propenylamino]-3-(4-nitrophenoxy)-2-propanol | |
| 81 | 1-[[(2′-Chloro[1,1′-biphenyl]-4-yl)methyl]-2- | 453 |
| | propenylamino]-3-(4-nitrophenoxy)-2-propanol | |
| 82 | 1-(4-Nitrophenoxy)-3-[2-propenyl[[2′- | 487 |
| | (trifluoromethyl)[1,1′-biphenyl]-4- | |
| | yl]methyl]amino]-2-propanol | |
| 83 | (α¹S)-α-[[[(2′-Methyl[1,1′-biphenyl]-4-yl)methyl]-2- | 358 |
| | propenylamino]methyl]benzenemethanol | |
| 84 | (α¹S)-α-[[[(2′-Chloro[1,1′-biphenyl]-4-yl)methyl]-2- | 378 |
| | propenylamino]methyl]benzenemethanol | |
| 85 | (2R)-3-[[(2′-Chloro[1,1′-biphenyl]-4-yl)methyl]-2- | 402 |
| | propenylamino]-2-hydroxypropyl butanoate | |
| 86 | (2R)-2-Hydroxy-3-[2-propenyl[[2′- | 436 |
| | (trifluoromethyl)[1,1′-biphenyl]-4- | |
| | yl]methyl]amino]propyl butanoate | |
| 87 | Methyl 2-hydroxy-2-methyl-3-[2-propenyl[[2′- | 408 |
| | (trifluoromethyl)[1,1′-biphenyl]-4- | |
| | yl]methyl]amino]propanoate | |
| 88 | 1-(3-Fluoro-4-nitrophenoxy)-3-[methyl[[2′- | 479 |
| | (trifluoromethyl)[1,1′-biphenyl]-4- | |
| | yl]methyl]amino]-2-propanol | |
| 89 | 1-(4-Iodophenoxy)-3-[methyl[[2′- | 542 |
| | (trifluoromethyl)[1,1′-biphenyl]-4- | |
| | yl]methyl]amino]-2-propanol | |
| 90 | 1-(3-Fluorophenoxy)-3-[methyl[[2′- | 434 |
| | (trifluoromethyl)[1,1′-biphenyl]-4- | |
| | yl]methyl]amino]-2-propanol | |
| 91 | Ethyl 4-[2-hydroxy-3-[methyl[[2′- | 487 |
| | (trifluoromethyl)[1,1′-biphenyl]-4- | |
| | yl]methyl]amino]propoxy]-benzenecarboximidate | |
| 92 | 1-[[(2′-Chloro[1,1′-biphenyl]-4- | 445 |
| | yl)methyl]methylamino]-3-(3-fluoro-4- | |
| | nitrophenoxy)-2-propanol | |
| 93 | 1-[[(2′-Chloro[1,1′-biphenyl]-4- | 445 |
| | yl)methyl]methylamino]-3-(2-fluoro-4- | |
| | nitrophenoxy)-2-propanol | |
| 94 | 1-[[(2′-Chloro[1,1′-biphenyl]-4- | 427 |
| | yl)methyl]methylamino]-3-(4-nitrophenoxy)-2- | |
| | propanol | |
| 95 | 1-[[(2′,3′-Dimethyl[1,1′-biphenyl]-4- | 376 |
| | yl)methyl]methylamino]-3-phenoxy-2-propanol | |
| 96 | 1-[[(2′,3′-Dimethyl[1,1′-biphenyl]-4- | 421 |
| | yl)methyl]methylamino]-3-(4-nitrophenoxy)-2- | |
| | propanol | |
| 97 | N,N-Diethyl-4-[3-[[(5′-fluoro-2′-methyl[1,1′- | 509 |
| | biphenyl]-4-yl)methyl]methylamino]-2- | |
| | hydroxypropoxy]-3-methoxybenzamide | |
| 98 | Ethyl 4-[3-[[(5′-fluoro-2′-methyl[1,1′-biphenyl]-4- | 451 |
| | yl)methyl]methylamino]-2- | |
| | hydroxypropoxy]benzenecarboximidate | |
| 99 | 4-[3-[[[4′-Chloro-2′-(trifluoromethyl)[1,1′-biphenyl]- | 579 |
| | 4-yl]methyl]methylamino]-2-hydroxypropoxy]- | |
| | N,N-diethyl-3-methoxybenzamide | |
| 100 | 2-[3-[[[4′-Chloro-2′-(trifluoromethyl)[1,1′-biphenyl]- | 493 |
| | 4-yl]methyl]methylamino]-2- | |
| | hydroxypropoxy]benzamide | |
| 101 | 1-[[[4′-Chloro-2′-(trifluoromethyl)[1,1′-biphenyl]-4- | 480 |
| | yl]methyl]methylamino]-3-(3-methoxyphenoxy)-2- | |
| | propanol | |
| 102 | 1-[[[4′-Chloro-2′-(trifluoromethyl)[1,1′-biphenyl]-4- | 489 |
| | yl]methyl]methylamino]-3-(1H-indol-5-yloxy)-2- | |
| | propanol | |
| 103 | Ethyl 4-[3-[[[4′-chloro-2′-(trifluoromethyl)[1,1′- | 521 |
| | biphenyl]-4-yl]methyl]methylamino]-2- | |
| | hydroxypropoxy]benzenecarboximidate | |
| 104 | 1-[[[4′-Chloro-2′-(trifluoromethyl)[1,1′-biphenyl]-4- | 450 |
| | yl]methyl]methylamino]-3-phenoxy-2-propanol | |
| 105 | 1-[[[4′-Chloro-2′-(trifluoromethyl)[1,1′-biphenyl]-4- | 495 |
| | yl]methyl]methylamino]-3-(4-nitrophenoxy)-2- | |
| | propanol | |
| 106 | 2-Fluoro-α-[[methyl[[2′-(trifluoromethyl)[1,1′- | 404 |
| | biphenyl]-4- | |
| | yl]methyl]amino]methyl]benzenemethanol | |
| 107 | α-[[[(2′-Chloro[1,1′-biphenyl]-4- | 370 |
| | yl)methyl]methylamino]methyl]-2- | |
| | fluorobenzenemethanol | |
| 108 | α-[[[(2′-Chloro-6′-methyl[1,1′-biphenyl]-4- | 366 |
| | yl)methyl]methylamino]methyl]benzenemethanol | |
| 109 | α-[[[(2′,5′-Dimethyl[1,1′-biphenyl]-4- | 364 |
| | yl)methyl]methylamino]methyl]-2- | |
| | fluorobenzenemethanol | |
| 110 | 4-Chloro-α-[[[(2′,5′-dimethyl[1,1′-biphenyl]-4- | 380 |
| | yl)methyl]methylamino]methyl]benzenemethanol | |
| 111 | α-[[Methyl[[4-(4-methyl-3- | 338 |
| | thienyl)phenyl]methyl]amino]methyl]benzenemethanol | |
| 112 | 1-(2-Fluoro-4-nitrophenoxy)-3-[[[3-fluoro-2′- | 497 |
| | (trifluoromethyl)[1,1′-biphenyl]-4- | |
| | yl]methyl]methylamino]-2-propanol | |
| 113 | 1-[[[3-Fluoro-2′-(trifluoromethyl)[1,1′-biphenyl]-4- | 479 |
| | yl]methyl]methylamino]-3-(4-nitrophenoxy)-2- | |
| | propanol | |
| 114 | 1-(4-Fluorophenoxy)-3-[[[3-fluoro-2′- | 452 |
| | (trifluoromethyl)[1,1′-biphenyl]-4- | |
| | yl]methyl]methylamino]-2-propanol | |
| 115 | α-[[[[3-Fluoro-2′-(trifluoromethyl)[1,1′-biphenyl]-4- | 404 |
| | yl]methyl]methylamino]methyl]benzenemethanol | |
| 116 | 2-Fluoro-α-[[[[3-fluoro-2′-(trifluoromethyl)[1,1′- | 422 |
| | biphenyl]-4- | |
| | yl]methyl]methylamino]methyl]benzenemethanol | |
| 117 | 4-Chloro-α-[[[[3-fluoro-2′-(trifluoromethyl)[1,1′- | 438 |
| | biphenyl]-4- | |
| | yl]methyl]methylamino]methyl]benzenemethanol | |
| 118 | 1-[[[2-Chloro-2′-(trifluoromethyl)[1,1′-biphenyl]-4- | 513 |
| | yl]methyl]methylamino]-3-(2-fluoro-4- | |
| | nitrophenoxy)-2-propanol | |
| 119 | 1-[[[2-Chloro-2′-(trifluoromethyl)[1,1′-biphenyl]-4- | 495 |
| | yl]methyl]methylamino]-3-(4-nitrophenoxy)-2- | |
| | propanol | |
| 120 | 1-[[[2-Chloro-2′-(trifluoromethyl)[1,1′-biphenyl]-4- | 468 |
| | yl]methyl]methylamino]-3-(4-fluorophenoxy)-2- | |
| | propanol | |
| 121 | α-[[[[2-Chloro-2′-(trifluoromethyl)[1,1′-biphenyl]-4- | 420 |
| | yl]methyl]methylamino]methyl]benzenemethanol | |
| 122 | α-[[[[2-Chloro-2′-(trifluoromethyl)[1,1′-biphenyl]-4- | 438 |
| | yl]methyl]methylamino]methyl]-2- | |
| | fluorobenzenemethanol | |
| 123 | 4-Chloro-α-[[[[2-chloro-2′-(trifluoromethyl)[1,1′- | 454 |
| | biphenyl]-4- | |
| | yl]methyl]methylamino]methyl]benzenemethanol | |
| 124 | α-[[[(2-Chloro[1,1′-biphenyl]-4- | 352 |
| | yl)methyl]methylamino]methyl]benzenemethanol | |
| 125 | 1-[[(2′-Chloro-5′-methoxy[1,1′-biphenyl]-4- | 475 |
| | yl)methyl]methylamino]-3-(2-fluoro-4- | |
| | nitrophenoxy)-2-propanol | |
| 126 | 1-[[(2′-Chloro-5′-methoxy[1,1′-biphenyl]-4- | 457 |
| | yl)methyl]methylamino]-3-(4-nitrophenoxy)-2- | |
| | propanol | |
| 127 | 1-[[(2′-Chloro-5′-methoxy[1,1′-biphenyl]-4- | 430 |
| | yl)methyl]methylamino]-3-(4-fluorophenoxy)-2- | |
| | propanol | |
| 128 | α-[[[(2′-Chloro-5′-methoxy[1,1′-biphenyl]-4- | 382 |
| | yl)methyl]methylamino]methyl]benzenemethanol | |
| 129 | α-[[[(2′-Chloro-5′-methoxy[1,1′-biphenyl]-4- | 400 |
| | yl)methyl]methylamino]methyl]-2- | |
| | fluorobenzenemethanol | |
| 130 | 4-Chloro-α-[[[(2′-chloro-5′-methoxy[1,1′-biphenyl]- | 416 |
| | 4-yl)methyl]methylamino]methyl]benzenemethanol | |
| 131 | α-[[[(2′-Chloro-5′-methoxy[1,1′-biphenyl]-4- | 450 |
| | yl)methyl]methylamino]methyl]-4- | |
| | (trifluoromethyl)benzenemethanol | |
| 132 | α-[[Methyl[[5-[2-(trifluoromethyl)phenyl]-2- | 376 |
| | furanyl]methyl]amino]methyl]benzenemethanol | |

1. A compound of formula I or a pharmaceutically acceptable salt thereof: 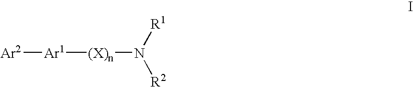
wherein
Ar¹ is arylene, heteroarylene, substituted arylene or substituted heteroarylene, wherein a ring atom of Ar¹ connected to Ar² is seperated from a ring atom of Ar¹ connected to X by at least one atom;
Ar² is aryl, heteroaryl, substituted aryl or substituted heteroaryl;
n is 0 or 1;
X is a divalent group that separates groups connected thereto by one or two atoms;
R¹ is a monovalent C₁₋₂₀ group comprising one or more heteroatoms selected from S, O, N and P;
R² is hydrogen, C₁₋₁₀ alkyl, C₁₋₁₀acyl, substituted C₁₋₁₀acyl, substituted C₁₋₁₀ alkyl, C₁₋₁₀ alkylene, or substituted C₁₋₁₀ alkylene, wherein said alkylene is linked to a ring carbon of Ar¹.

2. A compound according to claim 1, wherein
Ar¹ is an arylene, heteroarylene, substituted arylene or substituted heteroarylene, wherein a ring atom of Ar¹ connected to Ar² is seperated from a ring atom of Ar¹ connected to X by at least one atom;
Ar² is an aryl, heteroaryl, substituted aryl or substituted heteroaryl;
X is —CH₂—, or —CH₂—CH₂—;
R² is C₁₋₆ alkyl, substituted C₁₋₆ alkyl, C₁₋₃ alkylene, or substituted C₁₋₃ alkylene, wherein said alkylene is linked to a ring carbon of Ar¹.

3. A compound according to claim 2, wherein
R¹ is selected from: 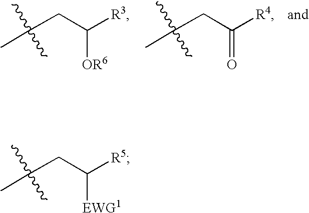
wherein R³ is optionally hydrogen, substituted C₁₋₁₀alkyl, optionally substituted C₅₋₁₂aryl, optionally substituted C₃₋₁₀heteroaryl, optionally substituted aryloxy-C₁₋₆alkyl, optionally substituted heteroaryloxy-C₁₋₆alkyl;
R⁴ and R⁵ are, independently, hydrogen, optionally substituted C₁₋₁₀alkyl, optionally substituted C₅₋₁₂aryl, optionally substituted C₃₋₁₀heteroaryl, amino group, —NHC(═O)—O—R⁷, or —NHC(═O)—R⁷, wherein R⁷ is C₁₋₆alkyl or aryl;
R⁶ is hydrogen, optionally substituted C₁₋₆alkyl, or optionally substituted aryl; and
EWG¹ is an electron withdrawing group.

4. A compound according to claim 1, wherein
Ar¹ is optionally substituted para-phenylene, optionally substituted six-membered para-heteroarylene, or optionally substituted monocyclic five-membered meta-heteroarylene;
Ar² is optionally substituted phenyl, or optionally substituted monocylic five or six-membered heteroaryl;
X is H₂—, or —CH₂—CH₂—;
R² is C₁₋₃ alkyl, substituted C₁₋₃ alkyl, C₁₋₃ alkylene, or substituted C₁₋₃ alkylene, wherein said alkylene is linked to a ring carbon of Ar¹.
R¹ is selected from: 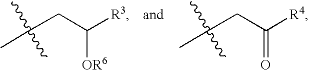
wherein R³ is optionally substituted C₁₋₆alkyl, optionally substituted phenyl, optionally substituted phenoxy-methyl;
R⁴ is, independently, optionally substituted C₁₋₆alkyl, optionally substituted phenyl, amino, —NHC(═O)—O—R⁷, or —NHC(═O)—R⁷, wherein R⁷ is C₁₋₆alkyl or phenyl; and
R⁶ is hydrogen, methyl or ethyl.

5. A compound according to claim 1, wherein
Ar¹ is para-phenylene or para-pyridylene;
Ar² is a phenyl ortho-substituted with an electron withdrawing group, or a thienyl ortho-substituted with an electron withdrawing group;
X is —CH₂—;
R² is methyl.
R¹ is selected from: 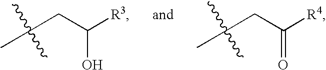
OH ,and 0
wherein R³ is optionally substituted phenyl, or optionally substituted phenoxy-methyl; and
R⁴ is —NHC(═O)—O—R⁷, wherein R⁷ is C₁₋₆alkyl.

6. A compound according to claim 5, wherein
Ar¹ is a phenyl ortho-substituted with —Cl, —F, —OMe, —OEt, —O—CH(CH₃)₂, —CF₃, —NO₂, or —CN; or thienyl ortho-substituted with —Cl, —F, —OMe, —OEt, —O—CH(CH₃)₂, —CF₃, —NO₂, —CN, wherein said ortho-subsituted Ar² is optionally further substituted at its non-ortho position; and
R³ is phenyl, substituted phenoxymethyl or substituted phenyl.

7. A compound of formula II, or a pharmaceutically acceptable salt thereof: 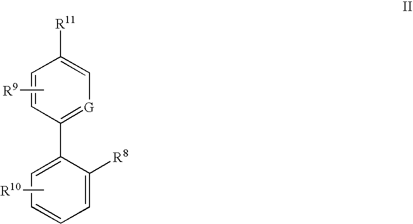
wherein
G is N or CH;
R⁸ is selected from —H, —CH₃, —CF₃, —NO₂ and —CN;
R⁹ is selected from —H and C₁₋₃alkyl;
R¹⁰ is selected from —H and C₁₋₃alkyl; and
R¹¹ is selected from 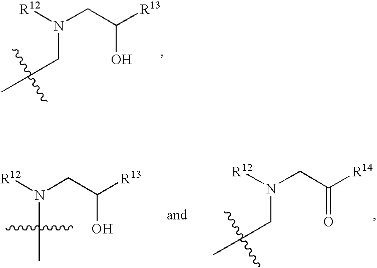
wherein R¹² is H or methyl, R¹³ is phenyl or substituted phenoxymethyl, R¹⁴ is —NHC(═O)OR¹⁵, wherein R¹⁵ is C₁₋₆alkyl.

8. A compound of formula III or IV, or a pharmaceutically acceptable salt thereof: 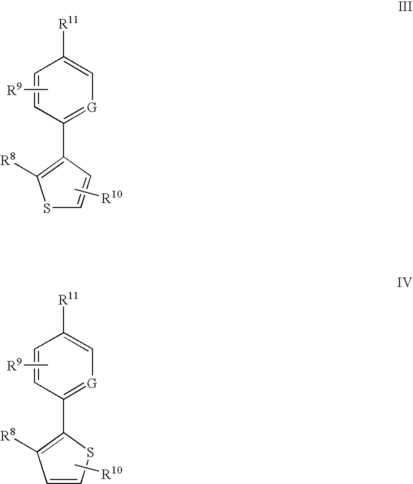
wherein
G is N or CH;
R⁸ is selected from —H, —CH₃, —CF₃, —NO₂ and —CN;
R⁹ is selected from —H and C₁₋₃alkyl;
R¹⁰ is selected from —H and C₁₋₃alkyl; and
R¹¹ is selected from 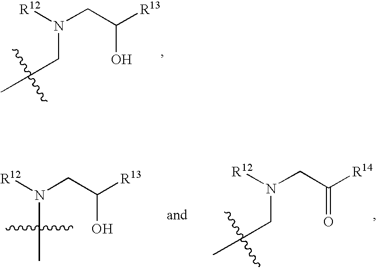
wherein R¹² is H or methyl, R¹³ is phenyl or substituted phenoxymethyl, R¹⁴ is —NHC(═O)OR¹⁵, wherein R¹⁵ is C₁₋₆alkyl.

9. A compound of formula V, or a pharmaceutically acceptable salt thereof: 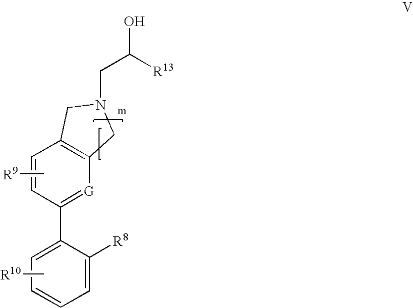
wherein
G is N or CH;
m is 1 or 2;
R⁸ is selected from —H, —CH₃, —CF₃, —NO₂ and —CN;
R⁹ is selected from —H and C₁₋₃alkyl;
R¹⁰ is selected from —H and C₁₋₃alkyl; and
R¹³ is phenyl or substituted phenoxymethyl.

10. A compound selected from:
α-[[Methyl[(2′-methyl[1,1′-biphenyl]-4-yl)methyl]amino]methyl]-benzenemethanol;
α-[[[(2′-Methoxy[1,1′-biphenyl]-4-yl)methyl]methylamino]methyl]-benzenemethanol;
α-[[[(2′-Chloro[1,1′-biphenyl]-4-yl)methyl]methylamino]methyl]-benzenemethanol;
α-[[Methyl-[[2′-(trifluoromethyl)-[1,1′-biphenyl]-4-yl]methyl]amino]methyl]-benzenemethanol;
1-(3,4-Dichlorophenoxy)-3-[methyl[[2′-(trifluoromethyl)[1,1′-biphenyl]-4-yl]methyl]amino]-2-propanol;
α-[(2-Fluoro-4-nitrophenoxy)methyl]-3,4-dihydro-6-[2-(trifluoromethyl)phenyl]-2(1H)-isoquinolineethanol;
Ethyl[[methyl-[[2′-(trifluoromethyl)-[1,1′-biphenyl]-4-yl]methyl]amino]-acetyl]carbamate;
3,4-Dihydro-α-phenyl-7-[2-(trifluoromethyl)phenyl]-2(1H)-isoquinolineethanol;
1-(2-Fluoro-4-nitrophenoxy)-3-[methyl[2′-(trifluoromethyl)[1,1′-biphenyl]-4-yl]amino]-2-propanol;
α-[(2-Fluoro-4-nitrophenoxy)methyl]-1,3-dihydro-5-[2-(trifluoromethyl)phenyl]-2H-isoindole-2-ethanol;
1-(2-Fluoro-4-nitrophenoxy)-3-[methyl[[2′-(trifluoromethyl)[1,1′-biphenyl]-4-yl]methyl]amino]-2-propanol;
α-[[Methyl-[[6-[2-(trifluoromethyl)phenyl]-3-pyridinyl]methyl]amino]methyl]-benzenemethanol;
α-[[Methyl[(2′-nitro[1,1′-biphenyl]-4-yl)methyl]amino]methyl]-benzenemethanol;
(α¹S)-α-[[Methyl[[2′-(trifluoromethyl)[1,1′-biphenyl]-4-yl]methyl]amino]methyl]-benzenemethanol;
(α¹R)-α-[[Methyl[[2′-(trifluoromethyl)[1,1′-biphenyl]-4-yl]methyl]amino]methyl]-benzenemethanol;
α-[[Methyl[[2-methyl-2′-(trifluoromethyl)[1,1′-biphenyl]-4-yl]methyl]amino]methyl]-benzenemethanol;
N-(2-Hydroxy-2-phenylethyl)-N-[[2′-(trifluoromethyl)[1,1′-biphenyl]-4-yl]methyl]acetamide;
N-(2-Hydroxy-2-phenylethyl)-N-methyl-2′-(trifluoromethyl)-[1,1′-biphenyl]-4-carboxamide;
β-Methoxy-N-methyl-N-[[2′-(trifluoromethyl)[1,1′-biphenyl]-4-yl]methyl]-benzeneethanamine;
3,4-Dihydro-α-phenyl-6-[2-(trifluoromethyl)phenyl]-2(1H)-isoquinolineethanol;
α-[[Methyl[[5-[1-methyl-5-(trifluoromethyl)-1H-pyrazol-3-yl]-2-thienyl]methyl]amino]methyl]-benzenemethanol;
1-(2-Fluoro-4-nitrophenoxy)-3-[methyl[[2′-(trifluoromethyl)[1,1′-biphenyl]-4-yl]methyl]amino]-2-propanol;
1-[Methyl[[2′-(trifluoromethyl)[1,1′-biphenyl]-4-yl]methyl]amino]-3-(4-nitrophenoxy)-2-propanol;
1-[[(2′,3′-Dimethyl[1,1′-biphenyl]-4-yl)methyl]methylamino]-3-(2-fluoro-4-nitrophenoxy)-2-propanol;
α-[[Methyl-[[2′-(trifluoromethyl)-[1,1′-biphenyl]-4-yl]methyl]amino]methyl]-benzenemethanol;
4-Chloro-α-[[[(2′-chloro[1,1′-biphenyl]-4-yl)methyl]methylamino]methyl]-benzenemethanol;
1-[[(2′,5′-Dimethyl[1,1′-biphenyl]-4-yl)methyl]methylamino]-3-(2-fluoro-4-nitrophenoxy)-2-propanol;
α-[[[(2′,5′-Dimethyl[1,1′-biphenyl]-4-yl)methyl]methylamino]methyl]-benzenemethanol;
α-[[Methyl[[4-(3-methyl-2-thienyl)phenyl]methyl]amino]methyl]-benzenemethanol;
1-[4-(1,1-Dimethylethyl)phenoxy]-3-[methyl[[2′-(trifluoromethyl)[1,1′-biphenyl]-4-yl]methyl]amino]-2-propanol;
1-[4-(1,1-Dimethylethyl)phenoxy]-3-[[(2′-methoxy[1,1′-biphenyl]-4-yl)methyl]methylamino]-2-propanol;
β-Ethoxy-N-methyl-N-[[2′-(trifluoromethyl)[1,1′-biphenyl]-4-yl]methyl]benzeneethanamine;
N-Methyl-N-[[2′-(trifluoromethyl)[1,1′-biphenyl]-4-yl]methyl]glycylglycine, ethyl ester;
N-Ethyl-2-[methyl[[2′-(trifluoromethyl)[1,1′-biphenyl]-4-yl]methyl]amino]acetamide;
α-[(2-Fluoro-4-nitrophenoxy)methyl]-3,4-dihydro-7-[2-(trifluoromethyl)phenyl]-2(1H)-isoquinolineethanol;
α-[[Methyl[(2,2′,5′-trimethyl[1,1′-biphenyl]-4-yl)methyl]amino]methyl]benzenemethanol;
1-[[[2′-Chloro-5′-(trifluoromethyl)[1,1′-biphenyl]-4-yl]methyl]methylamino]-3-(2-fluoro-4-nitrophenoxy)-2-propanol;
4′-[[[3-(2-Fluoro-4-nitrophenoxy)-2-hydroxypropyl]methylamino]methyl]-6-methoxy-[1,1′-biphenyl]-3-carbonitrile;
1-[[(2′,5′-Dichloro[1,1′-biphenyl]-4-yl)methyl]methylamino]-3-(2-fluoro-4-nitrophenoxy)-2-propanol;
1-[[[4-(2-Chloro-3-thienyl)phenyl]methyl]methylamino]-3-(2-fluoro-4-nitrophenoxy)-2-propanol;
4′-[[[3-(2-Fluoro-4-nitrophenoxy)-2-hydroxypropyl]methylamino]methyl]-[1,1′-biphenyl]-2-carbonitrile;
1-[[(2′-Chloro-5′-methyl[1,1′-biphenyl]-4-yl)methyl]methylamino]-3-(2-fluoro-4-nitrophenoxy)-2-propanol;
1-[[(5′-Chloro-2′-methyl[1,1′-biphenyl]-4-yl)methyl]methylamino]-3-(2-fluoro-4-nitrophenoxy)-2-propanol;
1-(2-Fluoro-4-nitrophenoxy)-3-[methyl[(2′-nitro[1,1′-biphenyl]-4-yl)methyl]amino]-2-propanol;
α-[[[[4-(2-Chloro-3-thienyl)phenyl]methyl]methylamino]methyl]benzenemethanol;
4′-[[(2-Hydroxy-2-phenylethyl)methylamino]methyl]-[1,1′-biphenyl]-2-carbonitrile;
α-[[[(5′-Chloro-2′-methyl[1,1′-biphenyl]-4-yl)methyl]methylamino]methyl]benzenemethanol;
α-[[Methyl[[2′-methyl-5′-(trifluoromethyl)[1,1′-biphenyl]-4-yl]methyl]amino]methyl]benzenemethanol;
α-[[[[2′-Chloro-5′-(trifluoromethyl)[1,1′-biphenyl]-4-yl]methyl]methylamino]methyl]benzenemethanol;
4′-[[(2-Hydroxy-2-phenylethyl)methylamino]methyl]-6-methoxy-[1,1′-biphenyl]-3-carbonitrile;
α-[[[(2′-Fluoro[1,1′-biphenyl]-4-yl)methyl]methylamino]methyl]benzenemethanol;
α-[[[(2′,5′-Dichloro[1,1′-biphenyl]-4-yl)methyl]methylamino]methyl]-benzenemethanol;
Methyl 3-[4-[[(2-hydroxy-2-phenylethyl)methylamino]methyl]phenyl]-2-thiophenecarboxylate;
α-[[Methyl[[2′-(1-methylethoxy)[1,1′-biphenyl]-4-yl]methyl]amino]methyl]benzenemethanol;
α-[[[(2′-Ethoxy[1,1′-biphenyl]-4-yl)methyl]methylamino]methyl]benzenemethanol;
α-[[Methyl[[2′-(2-propenyl)[1,1′-biphenyl]-4-yl]methyl]amino]methyl]benzenemethanol;
α-[[[(2′-Cyclopentyl[1,1′-biphenyl]-4-yl)methyl]methylamino]methyl]benzenemethanol;
α-[[Methyl[[5′-methyl-2′-(1-methylethyl)[1,1′-biphenyl]-4-yl]methyl]amino]methyl]benzenemethanol;
α-[[[(2′-Methoxy-5′-methyl[1,1′-biphenyl]-4-yl)methyl]methylamino]methyl]-benzenemethanol;
1-(2-Fluoro-4-nitrophenoxy)-3-[methyl[[2′-methyl-5′-(trifluoromethyl)[1,1′-biphenyl]-4-yl]methyl]amino]-2-propanol;
α-[[[[5-(4-Bromophenyl)-2-furanyl]methyl]methylamino]methyl]benzenemethanol;
α-[[[[5-(4-Chlorophenyl)-2-furanyl]methyl]methylamino]methyl]benzenemethanol;
α-[[Methyl[[5-[3-(trifluoromethyl)phenyl]-2-furanyl]methyl]amino]methyl]benzenemethanol;
Methyl 3-[5-[[(2-hydroxy-2-phenylethyl)methylamino]methyl]-2-furanyl]-2-thiophenecarboxylate;
α-[[Methyl[[4-(3-pyridinyl)phenyl]methyl]amino]methyl]benzenemethanol;
1-[[(2′-Chloro[1,1′-biphenyl]-4-yl)methyl]methylamino]-3-[4-(1,1-dimethylethyl)phenoxy]-2-propanol;
1-(4-Chlorophenoxy)-3-[methyl[[2′-(trifluoromethyl)[1,1′-biphenyl]-4-yl]methyl]amino]-2-propanol;
1-[Methyl[[2′-(trifluoromethyl)[1,1′-biphenyl]-4-yl]methyl]amino]-3-phenoxy-2-propanol;
1-[[(2′-Methoxy[1,1′-biphenyl]-4-yl)methyl]methylamino]-3-(4-nitrophenoxy)-2-propanol;
α-[[Methyl[[2′-(trifluoromethyl)[1,1′-biphenyl]-4-yl]methyl]amino]methyl]benzeneethanol;
1-(1,1-Dimethylethoxy)-3-[methyl[[2′-(trifluoromethyl)[1,1′-biphenyl]-4-yl]methyl]amino]-2-propanol;
Methyl 2-hydroxy-2-methyl-3-[methyl[[2′-(trifluoromethyl)[1,1′-biphenyl]-4-yl]methyl]amino]propanoate;
(β¹S)-β-[[(2′-Chloro[1,1′-biphenyl]-4-yl)methyl]methylamino]-cyclohexanepropanol;
1-(4-Chlorophenoxy)-3-[[(2′-methyl[1,1′-biphenyl]-4-yl)methyl]-2-propenylamino]-2-propanol;
1-[[(2′-Methyl[1,1′-biphenyl]-4-yl)methyl]-2-propenylamino]-3-phenoxy-2-propanol;
1-[[(2′-Chloro[1,1′-biphenyl]-4-yl)methyl]-2-propenylamino]-3-phenoxy-2-propanol;
1-Phenoxy-3-[2-propenyl[[2′-(trifluoromethyl)[1,1′-biphenyl]-4-yl]methyl]amino]-2-propanol;
1-[[(2′-Chloro[1,1′-biphenyl]-4-yl)methyl]-2-propenylamino]-3-(3,4-dichlorophenoxy)-2-propanol;
1-[([1,1′-Biphenyl]-4-ylmethyl)-2-propenylamino]-3-(4-nitrophenoxy)-2-propanol;
1-[[(2′-Methyl[1,1′-biphenyl]-4-yl)methyl]-2-propenylamino]-3-(4-nitrophenoxy)-2-propanol;
1-[[(2′-Chloro[1,1′-biphenyl]-4-yl)methyl]-2-propenylamino]-3-(4-nitrophenoxy)-2-propanol;
1-(4-Nitrophenoxy)-3-[2-propenyl[[2′-(trifluoromethyl)[1,1′-biphenyl]-4-yl]methyl]amino]-2-propanol;
(α¹S)-α-[[[(2′-Methyl[1,1′-biphenyl]-4-yl)methyl]-2-propenylamino]methyl]benzenemethanol;
(α¹S)-α-[[[(2′-Chloro[1,1′-biphenyl]-4-yl)methyl]-2-propenylamino]methyl]benzenemethanol;
(2R)-3-[[(2′-Chloro[1,1′-biphenyl]-4-yl)methyl]-2-propenylamino]-2-hydroxypropyl butanoate;
(2R)-2-Hydroxy-3-[2-propenyl[[2′(trifluoromethyl)[1,1′-biphenyl]-4-yl]methyl]amino]propyl butanoate;
Methyl 2-hydroxy-2-methyl-3-[2-propenyl[[2′-(trifluoromethyl)[1,1′-biphenyl]-4-yl]methyl]amino]propanoate;
1-(3-Fluoro-4-nitrophenoxy)-3-[methyl[[2′-(trifluoromethyl)[1,1′-biphenyl]-4-yl]methyl]amino]-2-propanol;
1-(4-Iodophenoxy)-3-[methyl[[2′-(trifluoromethyl)[1,1′-biphenyl]-4-yl]methyl]amino]-2-propanol;
1-(3-Fluorophenoxy)-3-[methyl[[2′-(trifluoromethyl)[1,1′-biphenyl]-4-yl]methyl]amino]-2-propanol;
Ethyl 4-[2-hydroxy-3-[methyl[[2′-(trifluoromethyl)[1,1′-biphenyl]-4-yl]methyl]amino]propoxy]-benzenecarboximidate;
1-[[(2′-Chloro[1,1′-biphenyl]-4-yl)methyl]methylamino]-3-(3-fluoro-4-nitrophenoxy)-2-propanol;
1-[[(2′-Chloro[1,1′-biphenyl]-4-yl)methyl]methylamino]-3-(2-fluoro-4-nitrophenoxy)-2-propanol;
1-[[(2′-Chloro[1,1′-biphenyl]-4-yl)methyl]methylamino]-3-(4-nitrophenoxy)-2-propanol;
1-[[(2′,3′-Dimethyl[1,1′-biphenyl]-4-yl)methyl]methylamino]-3-phenoxy-2-propanol;
1-[[(2′,3′-Dimethyl[1,1′-biphenyl]-4-yl)methyl]methylamino]-3-(4-nitrophenoxy)-2-propanol;
N,N-Diethyl-4-[3-[[(5′-fluoro-2′-methyl[1,1′-biphenyl]-4-yl)methyl]methylamino]-2-hydroxypropoxy]-3-methoxybenzamide;
Ethyl 4-[3-[[(5′-fluoro-2′-methyl[1,1′-biphenyl]-4-yl)methyl]methylamino]-2-hydroxypropoxy]benzenecarboximidate;
4-[3-[[[4′-Chloro-2′-(trifluoromethyl)[1,1′-biphenyl]-4-yl]methyl]methylamino]-2-hydroxypropoxy]-N,N-diethyl-3-methoxybenzamide;
2-[3-[[[4′-Chloro-2′-(trifluoromethyl)[1,1′-biphenyl]-4-yl]methyl]methylamino]-2-hydroxypropoxy]benzamide;
1-[[[4′-Chloro-2′-(trifluoromethyl)[1,1′-biphenyl]-4-yl]methyl]methylamino]-3-(3-methoxyphenoxy)-2-propanol;
1-[[[4′-Chloro-2′-(trifluoromethyl)[1,1′-biphenyl]-4-yl]methyl]methylamino]-3-(1H-indol-5-yloxy)-2-propanol;
Ethyl 4-[3-[[[4′-chloro-2′-(trifluoromethyl)[1,1′-biphenyl]-4-yl]methyl]methylamino]-2-hydroxypropoxy]benzenecarboximidate;
1-[[[4′-Chloro-2′-(trifluoromethyl)[1,1′-biphenyl]-4-yl]methyl]methylamino]-3-phenoxy-2-propanol;
1-[[[4′-Chloro-2′-(trifluoromethyl)[1,1′-biphenyl]-4-yl]methyl]methylamino]-3-(4-nitrophenoxy)-2-propanol;
2-Fluoro-α-[[methyl[[2′-(trifluoromethyl)[1,1′-biphenyl]-4-yl]methyl]amino]methyl]benzenemethanol;
α-[[[(2′-Chloro[1,1′-biphenyl]-4-yl)methyl]methylamino]methyl]-2-fluorobenzenemethanol;
α-[[[(2′-Chloro-6′-methyl[1,1′-biphenyl]-4-yl)methyl]methylamino]methyl]benzenemethanol;
α-[[[(2′,5′-Dimethyl[1,1′-biphenyl]-4-yl)methyl]methylamino]methyl]-2-fluorobenzenemethanol;
4-Chloro-α-[[[(2′,5′-dimethyl[1,1′-biphenyl]-4-yl)methyl]methylamino]methyl]benzenemethanol;
α-[[Methyl[[4-(4-methyl-3-thienyl)phenyl]methyl]amino]methyl]benzenemethanol;
1-(2-Fluoro-4-nitrophenoxy)-3-[[[3-fluoro-2′-(trifluoromethyl)[1,1′-biphenyl]-4-yl]methyl]methylamino]-2-propanol;
1-[[[3-Fluoro-2′-(trifluoromethyl)[1,1′-biphenyl]-4-yl]methyl]methylamino]-3-(4-nitrophenoxy)-2-propanol;
1-(4-Fluorophenoxy)-3-[[[3-fluoro-2′-(trifluoromethyl)[1,1′-biphenyl]-4-yl]methyl]methylamino]-2-propanol;
α-[[[[3-Fluoro-2′-(trifluoromethyl)[1,1′-biphenyl]-4-yl]methyl]methylamino]methyl]benzenemethanol;
2-Fluoro-α-[[[[3-fluoro-2′-(trifluoromethyl)[1,1′-biphenyl]-4-yl]methyl]methylamino]methyl]benzenemethanol;
4-Chloro-α-[[[[3-fluoro-2′-(trifluoromethyl)[1,1′-biphenyl]-4-yl]methyl]methylamino]methyl]benzenemethanol;
1-[[[2-Chloro-2′-(trifluoromethyl)[1,1′-biphenyl]-4-yl]methyl]methylamino]-3-(2-fluoro-4-nitrophenoxy)-2-propanol;
1-[[[2-Chloro-2′-(trifluoromethyl)[1,1′-biphenyl]-4-yl]methyl]methylamino]-3-(4-nitrophenoxy)-2-propanol;
1-[[[2-Chloro-2′-(trifluoromethyl)[1,1′-biphenyl]-4-yl]methyl]methylamino]-3-(4-fluorophenoxy)-2-propanol;
α-[[[[2-Chloro-2′-(trifluoromethyl)[1,1′-biphenyl]-4-yl]methyl]methylamino]methyl]benzenemethanol;
α-[[[[2-Chloro-2′-(trifluoromethyl)[1,1′-biphenyl]-4-yl]methyl]methylamino]methyl]-2-fluorobenzenemethanol;
4-Chloro-α-[[[[2-chloro-2′-(trifluoromethyl)[1,1′-biphenyl]-4-yl]methyl]methylamino]methyl]benzenemethanol;
α-[[[(2-Chloro[1,1′-biphenyl]-4-yl)methyl]methylamino]methyl]benzenemethanol;
1-[[(2′-Chloro-5′-methoxy[1,1′-biphenyl]-4-yl)methyl]methylamino]-3-(2-fluoro-4-nitrophenoxy)-2-propanol;
1-[[(2′-Chloro-5′-methoxy[1,1′-biphenyl]-4-yl)methyl]methylamino]-3-(4-nitrophenoxy)-2-propanol;
1-[[(2′-Chloro-5′-methoxy[1,1′-biphenyl]-4-yl)methyl]methylamino]-3-(4-fluorophenoxy)-2-propanol;
α-[[[(2′-Chloro-5′-methoxy[1,1′-biphenyl]-4-yl)methyl]methylamino]methyl]benzenemethanol;
α-[[[(2′-Chloro-5′-methoxy[1,1′-biphenyl]-4-yl)methyl]methylamino]methyl]-2-fluorobenzenemethanol;
4-Chloro-α-[[[(2′-chloro-5′-methoxy[1,1′-biphenyl]-4-yl)methyl]methylamino]methyl]benzenemethanol;
α-[[[(2′-Chloro-5′-methoxy[1,1′-biphenyl]-4-yl)methyl]methylamino]methyl]-4-(trifluoromethyl)benzenemethanol;
α-[[Methyl[[5-[2-(trifluoromethyl)phenyl]-2-furanyl]methyl]amino]methyl]benzenemethanol; and pharmaceutically acceptable salts thereof.

11-14. (canceled)

15. A pharmaceutical composition comprising a compound according to claim 1 and a pharmaceutically acceptable carrier.

16. A method for the therapy of pain in a warm-blooded animal, comprising the step of administering to said animal in need of such therapy a therapeutically effective amount of a compound according to claim 1.

17. A method for preparing a compound of formula X, 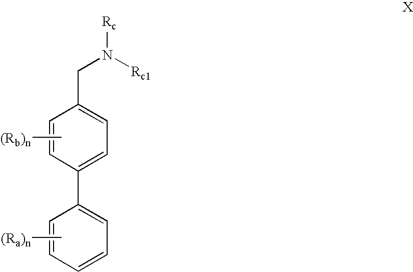
comprising the steps of
a) reacting a compound of formula IX with bis(pinacolato)diboron in the presence of Pd(PPh₃)₄; and 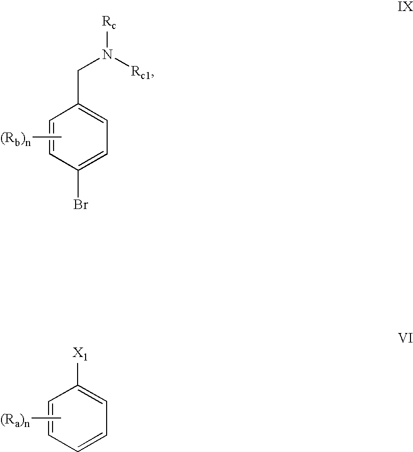
b) reacting a product of step a) with a compound of formula VI to form the compound of formula X,
wherein Rₐ and R_{b} are independently selected from —H, C₁₋₆alkyl, —CF₃, —NO₂, and —CN; n is 1 or 2; R_{c} is selected from: 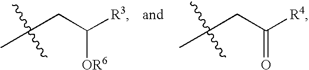
wherein R³ is optionally substituted phenyl, or optionally substituted phenoxy-methyl;
R⁴ is —NHC(═O)—O—R⁷, wherein R⁷ is C₁₋₆alkyl; and R_{c1} is —H or C₁₋₃alkyl.

18. A process for preparing a compound of formula XIII, 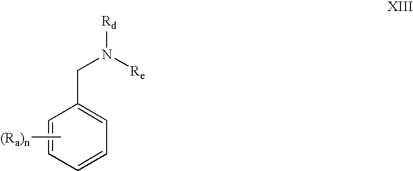
XIII
comprising the steps of:
a) reacting a compound of formula XI with R_{d}RₑNH; and 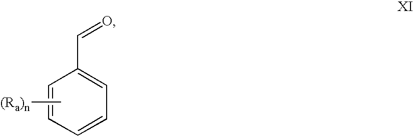
XI
b) reacting a product of step a) with NaBH(OAc)₃ to form the compound of formula XIII,
Rₐ is selected from optionally subsituted aryl, optionally subsituted heteroaryl; n is 1 or 2; R_{d} and Rₑ are independently selected from —H, C₁₋₃alkyl, 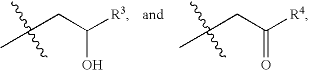
wherein R³ is optionally substituted phenyl, or optionally substituted phenoxy-methyl,
R⁴ is —NHC(═O)—O—R⁷, wherein R⁷ is C₁₋₆alkyl; wherein at least one of R_{d} and Rₑ contains an oxygen atom.

19. A method for preparing a compound of formula XV, 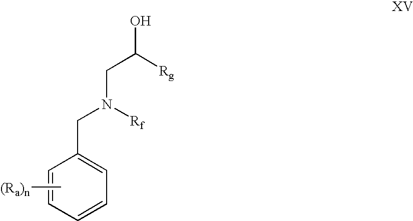
comprising the step of:
reacting a compound of formula XII with a compound of formula XIV, 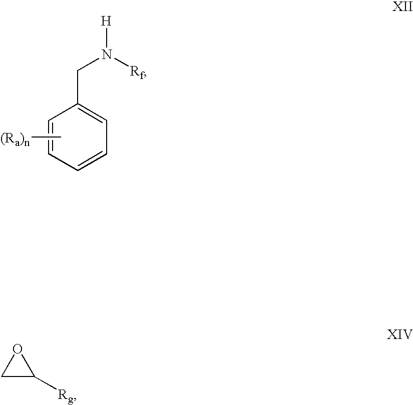
wherein Rₐ is selected from optionally substituted aryl and optionally substituted heteroaryl; n is 1 or 2; R_{f} is —H or C₁₋₃alkyl; and R_{g} is optionally subsitituted phenyl or optionally subsituted phenoxymethyl.
